(19) United States
(12) Patent Application Publication
(10) Pub. No.: US 2026/0257205 A1
(43) Pub. Date: Sep. 3, 2026

(54) PALLADIUM, PLATINUM, AND GOLD CATALYSTS FOR REVERSE WATER-GAS SHIFT AND INTEGRATED FISCHER-TROPSCH PROCESSES

(71) Applicants: BP P.L.C., London (GB); BP (CHINA) HOLDINGS LIMITED, Shanghai (CN)

(72) Inventors: Meiling Guo, Shanghai (CN); Gareth Armitage, York (GB); John Glenn Sunley, London (GB); Xuebin Liu, Shanghai (CN); Ben Dennis-Smither, London (GB); Christiana Udoh, London (GB); Eric Doskocil, Naperville, IL (US); Zai Hong Guan, Shanghai (CN); Alexander James Paterson, Hull (GB)

(21) Appl. No.: 18/878,272

(22) PCT Filed: Jun. 29, 2023
(86) PCT No.: PCT/IB2023/056802

§ 371 Date: Dec. 23, 2024

(30) Foreign Application Priority Data
Jun. 30, 2022 (WO) PCT/CN2022/102685
Jun. 30, 2022 (WO) PCT/CN2022/102763
Jun. 30, 2022 (WO) PCT/CN2022/102812
Jun. 30, 2022 (WO) PCT/CN2022/102976

(51) Int. Cl. B01J 23/68; B01J 21/04; B01J 21/06; B01J 23/10; B01J 23/656; B01J 37/02; B01J 37/08; B01J 37/18; C01B 32/40; C07C 1/04

(52) U.S. Cl. CPC B01J 23/688; B01J 21/04; B01J 21/063; B01J 23/10; B01J 23/6562; B01J 37/0201; B01J 37/0236; B01J 37/08; B01J 37/18; C01B 32/40; C07C 1/043

(57) ABSTRACT

The present disclosure relates generally to reverse water-gas shift processes, integrated Fischer-Tropsch processes, and supported reverse water-gas shift catalysts for conducting these processes. The catalysts described herein include a support that is a cerium oxide support, a titanium oxide support, an aluminum oxide support, a zirconium oxide support, or a mixed oxide support comprising a mixture of two or more of cerium oxide, titanium oxide, aluminum oxide, and zirconium oxide; at least one of platinum, palladium, and gold, present in an amount in the range of 0.05 to 10 wt % of the catalyst, based on the total weight of the catalyst; and manganese, present in an amount in the range of 0.5 to 20 wt % of the catalyst, based on the total weight of the catalyst.

### CROSS-REFERENCE TO RELATED APPLICATIONS

This application claims the benefit of priority of International Patent Application nos. PCT/CN2022/102685, filed Jun. 30, 2022, PCT/CN2022/102763, filed Jun. 30, 2022, PCT/CN2022/102976, filed Jun. 30, 2022, and PCT/CN2022/102812, filed Jun. 30, 2022, each of which is hereby incorporated herein by reference in its entirety.

### BACKGROUND OF THE DISCLOSURE

### 1. Field

The present disclosure relates generally to reverse water-gas shift catalysts, processes of making the same, and processes for performing reverse water-gas shift reactions. The present disclosure also relates to integrating processes for performing reverse water-gas shift reactions with processes for performing Fischer-Tropsch reactions.

### 2. Technical Background

The reverse water-gas shift reaction (rWGS) is an advantageous route to obtain carbon monoxide from carbon dioxide for further chemical processing. The rWGS converts carbon dioxide and hydrogen to carbon monoxide and water, as shown in Equation (1).

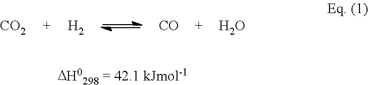

This can be used, for example, to modify the CO:H₂ ratio of a gas mixture for further processing. The carbon monoxide and hydrogen so formed is a valuable feedstock for a number of chemical processes, for example, the well-known Fischer-Tropsch (FT) process, shown in Equation (2).

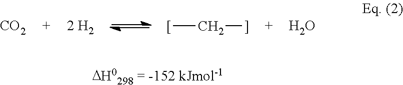

However, the rWGS reaction is not favored in all circumstances. For example, a competing reaction is the Sabatier reaction (Equation (3)), which decreases carbon monoxide yield in favor of methane production, which is not an active feedstock for FT.

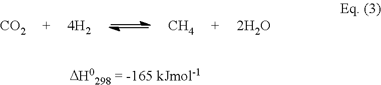

The strongly exothermic Sabatier reaction is thermodynamically favored over the endothermic rWGS reaction at lower reaction temperatures. As such, minimizing the methanation during rWGS, especially at low temperatures, can become a significant challenge.

Similarly, the carbon monoxide product from rWGS can be hydrogenated to methane, as shown in Equation (4).

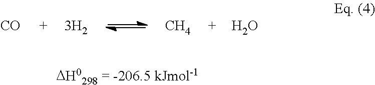

Hydrogenation of carbon monoxide to methane is also an exothermic reaction, so it too is favored at lower temperatures. The stoichiometry of the reaction requires at least a 3:1 ratio of hydrogen to carbon monoxide. This means that performing the rWGS reaction with a large excess of hydrogen to drive the equilibrium toward carbon monoxide (see Equation (1)) is not always ideal because it runs the risk of hydrogenating the carbon monoxide product to form methane.

Coupled with Equations (3) and (4), further undesirable side reactions can occur. These side reactions can form undesirable carbon deposits on the surface of catalysts used to promote rWGS. Examples of these carbon-producing side reactions are shown in Equations (5), (6), and (7). All three of these reactions are endothermic and are favored at higher temperatures, just like the rWGS reaction.

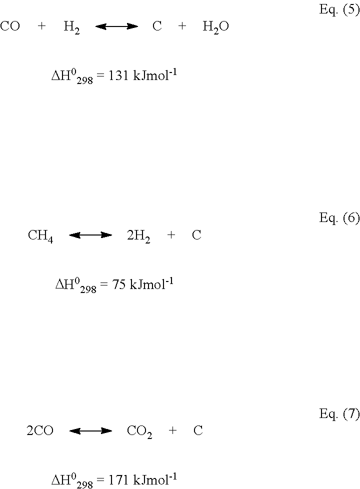

Accordingly, because the carbon-producing side reactions (Equations (5)-(7)) are also endothermic and are favored at higher temperatures, operation at higher temperatures to favor the desired carbon monoxide product can severely impact catalyst lifetime through the deposition of carbon.

Given the multiple reactions and competing thermodynamics at play, there remains a need in the art for new rWGS catalysts and processes, especially for integration with Fischer-Tropsch processes.

### SUMMARY

In one aspect, the present disclosure provides for a supported reverse water-gas shift catalyst comprising:
a support that is a cerium oxide support, a titanium oxide support, an aluminum oxide support, a zirconium oxide support, or a mixed oxide support comprising a mixture of two or more of cerium oxide, titanium oxide, aluminum oxide, and zirconium oxide;
at least one of platinum, palladium, and gold, present in an amount in the range of 0.05 to 10 wt % of the catalyst, based on the total weight of the catalyst; and
manganese, present in an amount in the range of 0.5 to 20 wt % of the catalyst, based on the total weight of the catalyst.

In another aspect, the present disclosure provides for a method of making the catalyst as described herein, the method comprising:
providing a support that is a cerium oxide support, a titanium oxide support, an aluminum oxide support, a zirconium support, or a mixed oxide support comprising a mixture of two or more of cerium oxide, titanium oxide, aluminum oxide and zirconium oxide;
contacting the support with a liquid comprising one or more platinum-, palladium-, or gold-containing compounds and one or more manganese-containing compounds dispersed in a solvent;
allowing the solvent to evaporate to provide a catalyst precursor; and
calcining the catalyst precursor.

In another aspect, the present disclosure provides for a catalyst as described herein made by the method as described herein.

In another aspect, the present disclosure provides a method for performing a reverse water-gas shift reaction, the method comprising contacting at a temperature in the range of 200-900° C. a catalyst as described herein with a feed stream comprising CO₂ and H₂, to provide a product stream comprising CO and H₂, the product stream having a lower concentration of CO₂ and a higher concentration of CO than the feed stream.

In one aspect, the present disclosure provides for a process for performing an integrated Fischer-Tropsch process, the process comprising:
providing a first feed stream comprising H₂ and CO₂;
contacting at a first temperature in the range of 200-900° C. and at a first pressure a reverse water-gas shift catalyst with the first feed stream to perform a reverse water-gas shift reaction to provide a first product stream comprising CO and H₂, the first product stream having a lower concentration of CO₂ and a higher concentration of CO than the first feed stream;
contacting at a second temperature and at a second pressure a Fischer-Tropsch catalyst with a second feed stream comprising H₂ and at least a portion of CO of the first product stream to provide a second product stream comprising C₅₊ hydrocarbons,
wherein the reverse water-gas shift catalyst is a supported reverse water-gas shift catalyst comprising:
   a support that is a cerium oxide support, a titanium oxide support, an aluminum oxide support, zirconium oxide support, or a mixed oxide support comprising a mixture of two or more of cerium oxide, titanium oxide, aluminum oxide, and zirconium oxide;
   at least one of platinum, palladium, and gold, present in an amount in the range of 0.05 to 10 wt % of the catalyst, based on the total weight of the catalyst; and
   manganese, present in an amount in the range of 0.5 to 20 wt % of the catalyst, based on the total weight of the catalyst.

### BRIEF DESCRIPTION OF FIGURES

The accompanying drawings are included to provide a further understanding of the methods of the disclosure, and are incorporated in and constitute a part of this specification. The drawings are not necessarily to scale, and sizes of various elements may be distorted for clarity. The drawings illustrate one or more embodiment(s) of the disclosure and together with the description serve to explain the principles and operation of the disclosure.

FIG. 1 is a schematic of the reverse water-gas shift reaction as described herein.

FIG. 2 is a schematic of a process for performing an integrated Fischer-Tropsch process as described herein.

FIG. 3 is a schematic of a process for performing an integrated Fischer-Tropsch process as described herein.

FIG. 4 is a schematic of a process for performing an integrated Fischer-Tropsch process as described herein.

FIG. 5 is a schematic of a process for performing an integrated Fischer-Tropsch process as described herein.

FIG. 6 is a schematic of a process for performing an integrated Fischer-Tropsch process as described herein.

FIG. 7 is a schematic of a process for performing an integrated Fischer-Tropsch process as described herein.

FIG. 8 is a graph of the conversion of carbon dioxide of the rWGS catalysts as described herein.

FIG. 9 is a graph of the carbon molar selectivity of methane of the rWGS catalysts as described herein.

FIG. 10 is a graph of the conversion of carbon dioxide of the rWGS catalysts as described herein.

FIG. 11 is a graph of the conversion of carbon molar selectivity of methane of the rWGS catalysts as described herein.

FIG. 12 is a graph of the conversion of carbon dioxide of the rWGS catalysts as described herein.

FIG. 13 is a graph of the conversion of carbon molar selectivity of methane of the rWGS catalysts as described herein.

### DETAILED DESCRIPTION

As discussed above, the reverse gas-water shift reaction reacts carbon dioxide with hydrogen to form carbon monoxide and water and can be useful in providing a feedstock containing carbon monoxide and hydrogen—often called “synthesis gas”—for use in processes such as the Fischer-Tropsch process. However, the Sabatier reaction, carbon monoxide methanation, and carbon-producing side reactions can interfere with the rWGS reaction. The Sabatier reaction and CO methanation are exothermic and favored at lower temperatures, while the rWGS and carbon-producing side reactions are endothermic and favored at higher temperatures. Accordingly, there remains a need for rWGS catalysts that can provide good performance in spite of these complicating factors. Here, the present inventors have provided supported reverse water-gas shift catalysts that include a metal oxide support, at least one or platinum, palladium, and gold, and manganese, that can meet the requirements necessary for a commercially-useful rWGS process. Additionally, the present inventors have found rWGS processes that are particularly advantageous for integration with a Fischer-Tropsch process by using supported reverse water-gas shift catalysts that include a metal oxide support, at least one of platinum, palladium, and gold, and manganese.

### Reverse Water-Gas Shift Catalyst

In one aspect, the present disclosure provides a supported reverse water-gas shift catalyst, for example, for use in an rWGS process integrated with an FT process. The supported reverse water-gas shift catalyst includes a support that is a cerium oxide support, a titanium oxide support, an aluminum oxide support, a zirconium oxide support, or a mixed oxide support including a mixture of two or more of cerium oxide, titanium oxide, aluminum oxide, and zirconium oxide; at least one of platinum, palladium, and gold, present in an amount in the range of 0.05 to 10 wt % of the catalyst, based on the total weight of the catalyst; and manganese, present in an amount in the range of 0.5 to 20 wt % of the catalyst, based on the total weight of the catalyst.

As described above, the reverse water-gas shift catalysts of the present disclosure are supported catalysts. In various embodiments as otherwise described herein, the support makes up at least 70 wt %, e.g., at least 75 wt %, or 80 wt %, or 85 wt %, or 90 wt % of the catalyst on an oxide basis.

In various embodiments as otherwise described herein, the support is a cerium oxide support. As used herein, a “cerium oxide” support is a support that presents at least a surface layer (e.g., 50 microns in thickness) that is at least 50 wt % cerium oxide, on an oxide basis. In various embodiments of the disclosure as described herein, at least a surface layer of the cerium oxide support includes at least 60 wt % cerium oxide, e.g., at least 70 wt % cerium oxide, or at least 80 wt % cerium oxide. In some such embodiments, at least a surface layer of the cerium oxide support includes at least 90 wt % cerium oxide. For example, in some embodiments, at least a surface layer of the cerium oxide support includes at least 95 wt % cerium oxide or at least 98 wt % cerium oxide. In various examples, the cerium oxide support contains cerium oxide substantially throughout, e.g., at least 50 wt % of the cerium oxide support is cerium oxide, on an oxide basis. For example, in various embodiments, the cerium oxide support includes at least 60 wt % cerium oxide, e.g., at least 70 wt % cerium oxide, or at least 80 wt % cerium oxide. In various embodiments, the cerium oxide support includes at least 90 wt % cerium oxide, e.g., at least 95 wt % cerium oxide, or at least 98 wt % cerium oxide. In some embodiments, the cerium oxide support may further include additional metals or metal oxides.

In various embodiments as otherwise described herein, the support is a titanium oxide support. As used herein, a “titanium oxide” support is a support that presents at least a surface layer (e.g., 50 microns in thickness) that is at least 50 wt % titanium oxide, on an oxide basis. In various embodiments of the disclosure as described herein, at least a surface layer of the titanium oxide support includes at least 60 wt % titanium oxide, e.g., at least 70 wt % titanium oxide, or at least 80 wt % titanium oxide. In some such embodiments, at least a surface layer of the titanium oxide support includes at least 90 wt % titanium oxide. For example, in some embodiments, at least a surface layer of the titanium oxide support includes at least 95 wt % titanium oxide or at least 98 wt % titanium oxide. In various examples, the titanium oxide support contains titanium oxide substantially throughout, e.g., at least 50 wt % of the titanium oxide support is titanium oxide, on an oxide basis. For example, in various embodiments, the titanium oxide support includes at least 60 wt % titanium oxide, e.g., at least 70 wt % titanium oxide, or at least 80 wt % titanium oxide. In various embodiments, the titanium oxide support includes at least 90 wt % titanium oxide, e.g., at least 95 wt % titanium oxide, or at least 98 wt % titanium oxide. In some embodiments, the titanium oxide support may further include additional metals or metal oxides.

In various embodiments as otherwise described herein, the support is an aluminum oxide support. As used herein, an “aluminum oxide” support is a support that presents at least a surface layer (e.g., 50 microns in thickness) that is at least 50 wt % aluminum oxide, on an oxide basis. In various embodiments of the disclosure as described herein, at least a surface layer of the aluminum oxide support includes at least 60 wt % aluminum oxide, e.g., at least 70 wt % aluminum oxide, or at least 80 wt % aluminum oxide. In some such embodiments, at least a surface layer of the aluminum oxide support includes at least 90 wt % aluminum oxide. For example, in some embodiments, at least a surface layer of the aluminum oxide support includes at least 95 wt % aluminum oxide or at least 98 wt % aluminum oxide. In various examples, the aluminum oxide support contains aluminum oxide substantially throughout, e.g., at least 50 wt % of the aluminum oxide support is aluminum oxide, on an oxide basis. For example, in various embodiments, the aluminum oxide support includes at least 60 wt % aluminum oxide, e.g., at least 70 wt % aluminum oxide, or at least 80 wt % aluminum oxide. In various embodiments, the aluminum oxide support includes at least 90 wt % aluminum oxide, e.g., at least 95 wt % aluminum oxide, or at least 98 wt % aluminum oxide. In some embodiments, the aluminum oxide support may further include additional metals or metal oxides.

In various embodiments as otherwise described herein, the support is a zirconium oxide support. As used herein, a “zirconium oxide” support is a support that presents at least a surface layer (e.g., 50 microns in thickness) that is at least 50 wt % zirconium oxide, on an oxide basis. In various embodiments of the disclosure as described herein, at least a surface layer of the zirconium oxide support includes at least 60 wt % zirconium oxide, e.g., at least 70 wt % zirconium oxide, or at least 80 wt % zirconium oxide. In some such embodiments, at least a surface layer of the zirconium oxide support includes at least 90 wt % zirconium oxide. For example, in some embodiments, at least a surface layer of the zirconium oxide support includes at least 95 wt % zirconium oxide or at least 98 wt % zirconium oxide. In various examples, the zirconium oxide support contains zirconium oxide substantially throughout, e.g., at least 50 wt % of the zirconium oxide support is zirconium oxide, on an oxide basis. For example, in various embodiments, the zirconium oxide support includes at least 60 wt % zirconium oxide, e.g., at least 70 wt % zirconium oxide, or at least 80 wt % zirconium oxide. In various embodiments, the zirconium oxide support includes at least 90 wt % zirconium oxide, e.g., at least 95 wt % zirconium oxide, or at least 98 wt % zirconium oxide. In some embodiments, the zirconium oxide support may further include additional metals or metal oxides.

In various embodiments as otherwise described herein, the support is a mixed oxide support. These can be provided, for example, by admixture of multiple of the oxides above and formation into a support that includes both. For example, in some embodiments, the mixed oxide support is a mixture of two or more metal oxides, such as cerium oxide, titanium oxide, aluminum oxide, and zirconium oxide. In some embodiments, at least a surface layer of the support includes at least 50 wt % total of two or more of cerium oxide, titanium oxide, aluminum oxide, and zirconium oxide, on an oxide basis. In some embodiments, at least a surface layer of the mixed oxide support includes at least 60 wt % total, e.g., at least 70 wt %, or at least 80 wt % of two or more of cerium oxide, titanium oxide, aluminum oxide, and zirconium oxide. In some embodiments, at least a surface layer of the mixed oxide support includes at least 90 wt %, e.g., at least 95 wt %, or at least 98 wt % of two or more cerium oxide, titanium oxide, aluminum oxide, and zirconium oxide. In various examples, the mixed oxide support contains the oxides substantially throughout, e.g., at least 50 wt % of the mixed oxide support is two or more of cerium oxide, titanium oxide, aluminum oxide, zirconium oxide. In various embodiments, the mixed oxide support includes at least 60 wt % total, e.g., at least 70 wt %, or at least 80 wt % of two or more of cerium oxide, titanium oxide, aluminum oxide, zirconium oxide. In various embodiments, the mixed oxide support includes at least 90 wt % total, e.g., at least 95 wt %, or at least 98 wt % of two or more of cerium oxide, titanium oxide, aluminum oxide, zirconium oxide. In some embodiments, the mixed oxide support may further include additional metals or metal oxides.

The present inventors have found that cerium oxide, titanium oxide, aluminum oxide, and zirconium oxide can provide good performance in the absence of substantial amounts of other metals in the support. For example, in various embodiments of the disclosure as otherwise described herein, the support does not include additional metals in a total amount of additional metals in excess of 2 wt %, e.g., in excess of 1 wt % or in excess of 0.5 wt %, on an oxide basis.

However, the inventors have noted that in many cases performance can be desirably effected by the inclusion of other metals in the support. Accordingly, in other embodiments as otherwise described herein, the support includes at least one additional metal. In various embodiments, the total amount of the at least one additional metal is in the range of 0.5-20 wt %, e.g., 1-20 wt %, or 2-20 wt %, or 0.5-15 wt %, or 1-15 wt %, or 2-15 wt %, or 0.5-10 wt %, or 1-10 wt %, or 2-10 wt %, or 0.5-5 wt %, or 1-5 wt %, on an oxide basis.

Supports suitable for use herein can be provided with a range of pore volumes. The person of ordinary skill in the art will select a pore volume appropriate for a desired catalytic process. For example, in various embodiments as otherwise described herein, the pore volume is at least 0.05 mL/g, e.g., at least 0.1 mL/g. In various embodiments as otherwise described herein, the pore volume is at most 1.5 mL/g, e.g., at most 1 mL/g. In various embodiments of the present disclosure as described herein, the pore volume is in the range of 0.05-1.5 mL/g, e.g., 0.1 mL/g to 1 mL/g. Pore volumes are measured by mercury porosimetry, for example, as measured according to ASTM D4284-12.

As described above, the supported reverse water-gas shift catalysts of the disclosure include at least one of platinum, palladium, and gold. For example, in various embodiments as otherwise described herein, platinum is present in the catalyst. For the purposes of this disclosure, the amount of platinum present is calculated as a weight percentage of platinum atoms in the catalyst based on the total weight of the catalyst, despite the form in which that platinum may be present. The platinum may be present in the catalyst in a variety of forms; most commonly, platinum is principally present as metal, metal oxide, or a combination thereof. In some embodiments of the present disclosure as described herein, platinum is present in the catalyst in an amount in the range of 0.05 to 10 wt %, e.g., in the range of 0.1 to 10 wt %, or 0.5 to 10 wt %, 1 to 10 wt %, or 2 to 10 wt %, or 5 to 10 wt %, based on the total weight of the catalyst. For example, in some embodiments, platinum is present in the catalyst in an amount in the range of 0.05 to 7 wt %, e.g., in the range of 0.1 to 7 wt %, or 0.5 to 7 wt %, or 1 to 7 wt %, or 2 to 7 wt %, based on the total weight of the catalyst. In some embodiments, platinum is present in the catalyst in an amount in the range of 0.05 to 5 wt %, e.g., in the range of 0.1 to 5 wt %, or 0.5 to 5 wt %, or 1 to 5 wt %, or 2 to 5 wt %, based on the total weight of the catalyst. For example, in some embodiments of the present disclosure as described herein, platinum is present in the catalyst in an amount in the range of 0.05 to 2 wt %, e.g., in the range of 0.1 to 2 wt %, or 0.3 to 2 wt %, or 0.5 to 2 wt %, based on the total weight of the catalyst. In some embodiments, platinum is present in the catalyst in an amount in the range of 0.05 to 1.5 wt %, e.g., in the range of 0.1 to 1.5 wt %, or 0.3 to 1.5 wt %, or 0.5 to 1.5 wt %, based on the total weight of the catalyst. In some embodiments, platinum is present in an amount in the range of 0.05 to 1 wt %, e.g., in the range of 0.1 to 1 wt %, or 0.3 to 1 wt %, or 0.5 to 1 wt %, based on the total weight of the catalyst. In some embodiments, platinum is present in the catalyst in an amount in the range of 0.05 to 0.8 wt %, e.g., in the range of 0.1 to 0.8 wt %, or 0.3 to 0.8 wt %, or 0.5 to 0.8 wt %, based on the total weight of the catalyst.

In various embodiments as otherwise described herein, palladium is present in the catalyst. For the purposes of this disclosure, the amount of palladium present is calculated as a weight percentage of palladium atoms in the catalyst based on the total weight of the catalyst, despite the form in which that palladium may be present. The palladium may be present in the catalyst in a variety of forms; most commonly, palladium is principally present as metal, metal oxide, or a combination thereof. In some embodiments of the present disclosure as described herein, palladium is present in the catalyst in an amount in the range of 0.05 to 10 wt %, e.g., in the range of 0.1 to 10 wt %, or 0.5 to 10 wt %, 1 to 10 wt %, or 2 to 10 wt %, or 5 to 10 wt %, based on the total weight of the catalyst. For example, in some embodiments, palladium is present in the catalyst in an amount in the range of 0.05 to 7 wt %, e.g., in the range of 0.1 to 7 wt %, or 0.5 to 7 wt %, or 1 to 7 wt %, or 2 to 7 wt %, based on the total weight of the catalyst. In some embodiments, palladium is present in the catalyst in an amount in the range of 0.05 to 5 wt %, e.g., in the range of 0.1 to 5 wt %, or 0.5 to 5 wt %, or 1 to 5 wt %, or 2 to 5 wt %, based on the total weight of the catalyst. For example, in some embodiments of the present disclosure as described herein, palladium is present in the catalyst in an amount in the range of 0.05 to 2 wt %, e.g., in the range of 0.1 to 2 wt %, or 0.3 to 2 wt %, or 0.5 to 2 wt %, based on the total weight of the catalyst. In some embodiments, palladium is present in the catalyst in an amount in the range of 0.05 to 1.5 wt %, e.g., in the range of 0.1 to 1.5 wt %, or 0.3 to 1.5 wt %, or 0.5 to 1.5 wt %, based on the total weight of the catalyst. In some embodiments, palladium is present in an amount in the range of 0.05 to 1 wt %, e.g., in the range of 0.1 to 1 wt %, or 0.3 to 1 wt %, or 0.5 to 1 wt %, based on the total weight of the catalyst. In some embodiments, palladium is present in the catalyst in an amount in the range of 0.05 to 0.8 wt %, e.g., in the range of 0.1 to 0.8 wt %, or 0.3 to 0.8 wt %, or 0.5 to 0.8 wt %, based on the total weight of the catalyst.

For example, in various embodiments as otherwise described herein, gold is present in the catalyst. For the purposes of this disclosure, the amount of gold present is calculated as a weight percentage of gold atoms in the catalyst based on the total weight of the catalyst, despite the form in which that gold may be present. The gold may be present in the catalyst in a variety of forms; most commonly, gold is principally present as metal, metal oxide, or a combination thereof. In some embodiments of the present disclosure as described herein, gold is present in the catalyst in an amount in the range of 0.05 to 10 wt %, e.g., in the range of 0.1 to 10 wt %, or 0.5 to 10 wt %, 1 to 10 wt %, or 2 to 10 wt %, or 5 to 10 wt %, based on the total weight of the catalyst. For example, in some embodiments, gold is present in the catalyst in an amount in the range of 0.05 to 7 wt %, e.g., in the range of 0.1 to 7 wt %, or 0.5 to 7 wt %, or 1 to 7 wt %, or 2 to 7 wt %, based on the total weight of the catalyst. In some embodiments, gold is present in the catalyst in an amount in the range of 0.05 to 5 wt %, e.g., in the range of 0.1 to 5 wt %, or 0.5 to 5 wt %, or 1 to 5 wt %, or 2 to 5 wt %, based on the total weight of the catalyst. For example, in some embodiments of the present disclosure as described herein, gold is present in the catalyst in an amount in the range of 0.05 to 2 wt %, e.g., in the range of 0.1 to 2 wt %, or 0.3 to 2 wt %, or 0.5 to 2 wt %, based on the total weight of the catalyst. In some embodiments, gold is present in the catalyst in an amount in the range of 0.05 to 1.5 wt %, e.g., in the range of 0.1 to 1.5 wt %, or 0.3 to 1.5 wt %, or 0.5 to 1.5 wt %, based on the total weight of the catalyst. In some embodiments, gold is present in an amount in the range of 0.05 to 1 wt %, e.g., in the range of 0.1 to 1 wt %, or 0.3 to 1 wt %, or 0.5 to 1 wt %, based on the total weight of the catalyst. In some embodiments, gold is present in the catalyst in an amount in the range of 0.05 to 0.8 wt %, e.g., in the range of 0.1 to 0.8 wt %, or 0.3 to 0.8 wt %, or 0.5 to 0.8 wt %, based on the total weight of the catalyst.

As described above, the supported reverse water-gas shift catalysts of the disclosure also include manganese. The present inventors have determined that inclusion of manganese in the catalyst can provide improved performance, as described in the Examples below. For the purposes of this disclosure, the amount of manganese present is calculated as a weight percentage of manganese atoms in the catalyst based on the total weight of the catalyst, despite the form in which that manganese may be present. The manganese may be present in the catalyst in a variety of forms; most commonly, manganese is principally present as metal oxide, metal, or a combination thereof. In various embodiments of the present disclosure as otherwise described herein, manganese is present in the catalyst in an amount in the range of 0.5 to 20 wt %, based on total weight of the catalyst. For example, in various embodiments, manganese is present in the catalyst in an amount in the range of 0.5 to 15 wt %, or 0.5 to 12 wt %, or 0.5 to 10 wt %, based on the total weight of the catalyst. In various embodiments of the present disclosure as described herein, manganese is present in the catalyst in an amount in the range of 1 to 20 wt %, e.g., in the range of 1 to 15 wt %, or 1 to 12 wt %, or 1 to 10 wt %, based on the total weight of the catalyst. In various embodiments of the present disclosure as described herein, manganese is present in an amount in the range of 2 to 20 wt %, e.g., in the range of 2 to 15 wt %, or 2 to 12 wt %, or 2 to 10 wt %, based on the total weight of the catalyst. In various embodiments of the present disclosure as described herein, manganese is present in an amount in the range of 4 to 20 wt %, e.g., in the range of 4 to 15 wt %, or 4 to 12 wt %, or 4 to 10 wt %, based on the total weight of the catalyst.

The platinum, palladium, and/or gold and the manganese can be provided in a variety of weight ratios. For example, in some embodiments of the present disclosure as described herein, the weight ratio of platinum, palladium, and/or gold to manganese present in the catalyst is at least 0.05:1. For example, in various embodiments, the weight ratio of platinum, palladium, and/or gold to manganese is at least 0.1:1. In various embodiments of the present disclosure as described herein, the weight ratio of platinum, palladium, and/or gold to manganese present in the catalyst is at most 1:1. For example, the weight ratio of platinum, palladium, and/or gold to manganese is at most 0.5:1. For example, in various embodiments, the weight ratio of platinum, palladium, and/or gold to manganese present in the catalyst is in the range of 0.05:1 to 1:1. For example, the weight ratio of platinum, palladium, and/or gold to manganese is in the range of 0.05:1 to 0.5:1, or 0.05:1 to 0.3:1, or 0.07:1 to 1:1, or 0.07:1 to 0.5:1, or 0.07:1 to 0.3:1, or 0.1:1 to 1:1, or 0.1:1 to 0.5:1, or 0.1:1 to 0.3:1.

The present inventors have determined that suitable reverse water-gas shift catalysts can be formed of one or more of cerium oxide, titanium oxide, aluminum oxide, and zirconium oxide as a support, with platinum, palladium, and/or gold in combination with manganese included in/on the catalyst. As would be understood by the person of ordinary skill in the art, the amount of cerium, titanium, aluminum, zirconium, platinum, palladium, gold, and manganese can be quantified on a metallic basis regardless of the form in which these metals may be present. For example, the amount of these metals can be calculated as a weight percentage based on the total weight of metals in the catalysts (i.e., on a metallic basis), without the inclusion of oxygen or non-metallic counterions in the calculation. Accordingly, in various embodiments of the present disclosure as described herein, the total amount of cerium, titanium, aluminum, zirconium, manganese, platinum, palladium, and gold in the catalyst is at least 90 wt %, e.g., at least 95 wt %, or at least 98 wt % of the catalyst, on a metallic basis. For example, in some particular embodiments, the total amount of cerium, manganese, platinum, palladium, and gold in the catalyst is at least 90 wt %, e.g., at least 95 wt %, or at least 98 wt % of the catalyst, on a metallic basis. In other embodiments, the total amount of titanium, manganese, platinum, palladium, and gold in the catalyst is at least 90 wt %, e.g., at least 95 wt %, or at least 98 wt % of the catalyst, on a metallic basis. In other embodiments, the total amount of aluminum, manganese, platinum, palladium, and gold in the catalyst is at least 90 wt %, e.g., at least 95 wt %, or at least 98 wt % of the catalyst, on a metallic basis. In other embodiments, the total amount of zirconium, manganese, platinum, palladium, and gold in the catalyst is at least 90 wt %, e.g., at least 95 wt %, or at least 98 wt % of the catalyst, on a metallic basis.

As described above, the catalyst as described herein is mostly composed of cerium, titanium, aluminum, zirconium, manganese, platinum, palladium, and/or gold. In some embodiments as described herein, the catalyst has an additional metal content of no more than 10 wt %, based on the total weight of the catalyst. The additional metal may be any metal that is not platinum, palladium, gold, manganese, cerium, titanium, aluminum, or zirconium. For example, the additional metal may be selected from alkali metals, alkaline earth-metals, rare-earth metals, coinage metals, or other transition metals. For example, in various embodiments, the catalyst has an additional metal content of no more than 5 wt %, or no more than 2 wt %, or no more than 1 wt %, based on the total weight of the catalyst. In some embodiments as described herein, the catalyst has a copper content of no more than 10 wt %, based on the total weight of the catalyst. For example, in various embodiments, the catalyst has a copper content of no more than 5 wt %, or no more than 2 wt %, or no more than 1 wt %, based on the total weight of the catalyst. In some embodiments as described herein, the catalyst has an alkali metal content of no more than 10 wt %, based on the total weight of the catalyst. For example, in various embodiments, the catalyst has an alkali metal content of no more than 5 wt %, or no more than 2 wt %, or no more than 1 wt %, based on the total weight of the catalyst. In some embodiments as described herein, the catalyst has an alkaline-earth metal content of no more than 10 wt %, based on the total weight of the catalyst. For example, in various embodiments, the catalyst has an alkaline-earth metal content of no more than 5 wt %, or no more than 2 wt %, or no more than 1 wt %, based on the total weight of the

As described above, the supported catalyst includes manganese and at least one of platinum, palladium, and gold. Depending on the process of synthesis, these species, which will typically be principally present in metallic form and/or oxide form, can be disposed at a variety of different places on the support. For example, they can be found in pores of the support and on the outer surface of the support. They may be found substantially throughout the support, e.g., as when a large volume of impregnation liquid is used, or only in a surface layer of the support, e.g., when impregnation liquid does not infiltrate into the entirety of the support, such as when using an incipient wetness technique.

Without intending to be bound by theory, it is believed that the active form of platinum, palladium, and gold is typically a substantially metallic form. As described below, as platinum, palladium, and gold may be present substantially in an oxide form after catalyst preparation and during shipment and storage, it is typically desirable to activate the catalyst by contacting it with a reductant, e.g., hydrogen gas, to convert a substantial fraction of such oxide to metallic form. However, the person of ordinary skill in the art will appreciate that the present disclosure contemplates the usefulness of a wide variety of palladium, platinum, and gold forms in its catalysts, as these can be active or can be conveniently transformed to active forms.

The manganese will typically be provided in oxide form after catalyst preparation and during shipment and storage. Without intending to be bound by theory, the present inventors believe that the manganese acts to improve the catalytic activity of the supported platinum, palladium, and/or gold catalysts by reducing CO methanation that can occur over the typical reverse water-gas shift reaction temperature range, which impacts CO selectivity. The present inventors believe that the improved activity can be attributed to the manganese interfacing with both the platinum, palladium, and/or gold and the support (e.g., cerium oxide, titanium oxide, aluminum oxide, zirconium oxide, or a mixed oxide). The present inventors contemplate that it is possible that some manganese oxide is converted to metallic form during the activation of the platinum, palladium, and/or gold species. However, the person of ordinary skill in the art will appreciate that the present disclosure contemplates the usefulness of a wide variety of manganese forms in its catalysts, as these can provide a promoting effect or can be conveniently transformed to forms that will.

The person of ordinary skill in the art will appreciate that the catalysts of the disclosure can be provided in many forms, depending especially on the particular form of the reactor system in which they are to be used, e.g., in a fixed bed or as a fluid bed. The supports themselves can be provided as discrete bodies of material, e.g., as porous particles, pellets or shaped extrudates, with platinum, palladium, and/or gold and manganese provided thereon to provide the catalyst. However, in other embodiments, a catalyst of the disclosure can itself be formed as a layer on an underlying substrate. The underlying substrate is not particularly limited. It can be formed of, e.g., a metal or metal oxide, and can itself be provided in a number of forms, such as particles, pellets, shaped extrudates, or monoliths. The person of ordinary skill in the art can, e.g., use coating or other forming techniques to provide a layer of support on the substrate, then add platinum, palladium, and/or gold and manganese.

Another aspect of the present disclosure provides for a method of making the catalyst as described herein. As described above, the method includes providing a support that is a cerium oxide support, a titanium oxide support, an aluminum oxide support, a zirconium oxide support, or a mixed oxide support including a mixture of two or more of cerium oxide, titanium oxide, aluminum oxide, and zirconium oxide; contacting the support with one or more liquids each including one or more platinum-, palladium-, or gold-containing compounds and/or one or more manganese-containing compounds dispersed in a solvent; allowing the solvent(s) to evaporate to provide a catalyst precursor; and calcining the catalyst precursor. The person of ordinary skill in the art will appreciate, of course, that other methods can be used to make the catalysts described herein.

In some embodiments of the present disclosure as described herein, contacting the support with the liquid includes adding the liquid in an amount about equal to (i.e., within 25% of, or within 10% of) the pore volume of the support. In other embodiments, contacting the support with the liquid includes adding the liquid in an amount greater than the pore volume of the support. For example, in some embodiments, the ratio of the amount of liquid to the amount of support on a mass basis is in the range of 0.75:1 to 5:1, e.g., in the range of 0.9:1 to 3:1. In some embodiments, contacting the support with the liquid provides a slurry.

In various embodiments of the present disclosure as described herein, allowing the solvent to evaporate is conducted at ambient temperature. In various embodiments, allowing the solvent to evaporate is conducted at an elevated temperature for a drying time. The person of ordinary skill in the art would be able to select appropriate apparatuses or instruments to allow the solvent to evaporate, and such apparatuses or instruments are not particularly limited. Additionally, the person of ordinary skill in the art would understand that the elevated temperature that will allow the solvent to evaporate depends on the boiling point of the solvent. As such, the person of ordinary skill in the art would be able to select an appropriate elevated temperature. For example, in some embodiments, the elevated temperature is in the range of 50-150° C., e.g., in the range of 50-120° C., or 50-100° C., or 100-150° C., or 100-120° C. In some embodiments, the drying time is in the range of 1 to 48 hours, e.g., in the range of 10 to 36 hours, or 12 to 24 hours. For example, in particular embodiments, the drying time is about 24 hours. In some embodiments, allowing the solvent to evaporate is conducted under vacuum and at an elevated temperature for a drying time, as described herein. In some embodiments, allowing the solvent to evaporate is conducted in a stirring drybath at an elevated temperature, for example, in the range of 30-100° C.

In some embodiments of the present disclosure as described herein, calcining the catalyst precursor is conducted in a furnace for a calcining time and at a calcining temperature. For example, in some embodiments, the calcining time is in the range of 0.5 to 24 hours, or 0.5 to 15 hours, or 0.5 to 10 hours, or 0.5 to 5 hours. In some embodiments, the calcining temperature is in the range of 100-600° C., e.g., in the range of 120-500° C.

As described above, the method of making the catalyst as described herein includes contacting the support with one or more liquids each including one or more platinum-, palladium, or gold-containing compounds and/or one or more manganese-containing compounds dispersed in a solvent. The platinum-, palladium-, gold-, and manganese-containing compounds are not particularly limited and the person of ordinary skill in the art would be able to choose appropriate compounds that are soluble in the solvent. For example, in some embodiments of the disclosure as described herein, the platinum-, palladium-, gold-, and manganese-containing compounds may be selected from metal salts (e.g., nitrates and acetates). The solvent is also not particularly limited and the person of ordinary skill in the art would be able to choose an appropriate solvent that can be absorbed by the support. For example, in some embodiments of the disclosure as described herein, the solvent is water. As the person of ordinary skill in the art will appreciate, these metal species are conveniently provided in the same liquid, so that only one step of contacting the support with liquid is required. However, other schemes are possible.

In another aspect, the present disclosure provides a catalyst as described herein made by the methods as described herein.

### Reverse Water-Gas Shift Reaction

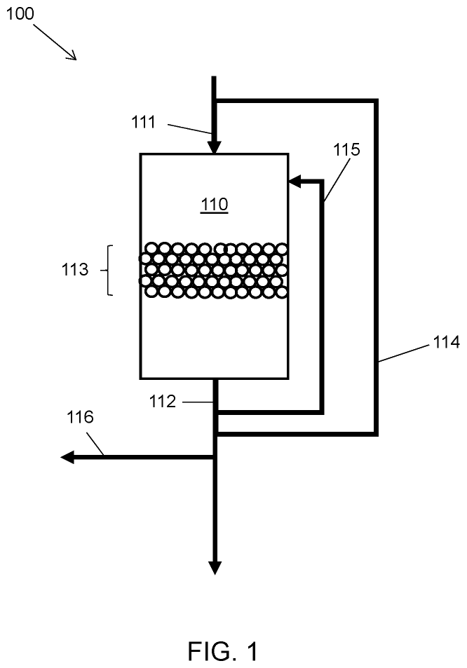

Another aspect of the present disclosure provides a method for performing a reverse water-gas shift reaction. As described above, the method includes contacting at a temperature in the range of 200-900° C. a catalyst as described herein with a feed stream that includes CO₂ and H₂, to provide a product stream that includes CO and H₂, the product stream having a lower concentration of CO₂ and a higher concentration of CO than the feed stream. An example of such a method is shown schematically in FIG. 1. In FIG. 1, the method 100 includes performing a reverse water-gas shift reaction by providing a feed stream 111 comprising H₂ and CO₂, here, to a reaction zone, e.g., a reactor 110. A reverse water-gas shift catalyst 113, as described herein, is contacted at a temperature in the range of 200-900° C. with the feed stream 111 to provide a product stream 112 comprising CO and H₂. The product stream has a lower concentration of CO₂ and a higher concentration of CO than the feed stream.

As used herein, a “feed stream” is used to mean the total material input to a process step, regardless of whether provided in a single physical stream or multiple physical streams, and whether through a single inlet or multiple inlets. For example, H₂ and CO of the feed stream can be provided to the reverse water-gas shift catalyst in a single physical stream (e.g., in a single pipe to reactor 110), or in multiple physical streams (e.g., separate inlets for CO and H₂, or one inlet for fresh CO and H₂ and another for recycled CO and/or H₂). Similarly, a “product stream” is used to mean the total material output from a process step, regardless of whether provided in a single physical stream or multiple physical streams, and whether through a single outlet or multiple outlets.

In various embodiments of the present disclosure as described herein, the reverse water-gas shift reaction has a CO selectivity of at least 95%, e.g., or at least 96%. As used herein, a “selectivity” for a given reaction product is the molar fraction of the feed (here, CO₂) that is converted to the product (for “CO selectivity,” CO). The present inventors have determined that the present catalysts, even when operating at lower temperatures than many conventional reverse water-gas shift catalysts, can provide excellent selectivity for CO, despite the potential for competition by the Sabatier reaction and the methanation of CO. For example, in some embodiments as otherwise described herein, the reverse water-gas shift reaction has a CO selectivity of at least 98%, e.g., or at least 99%.

Notably, even at relatively low temperatures in the range of 200-900° C., the catalysts described herein can be operated to provide carbon monoxide with only a very minor degree of methane formation. For example, in various embodiments of the present disclosure as otherwise described herein, the reverse water-gas shift reaction has a methane selectivity of no more than 5%, e.g., no more than 4%. For example, in some embodiments, the reverse water-gas shift reaction has a methane selectivity of no more than 2%, e.g., no more than 1%. In some embodiments, the reverse water-gas shift reaction has a methane selectivity of no more than 0.5%, e.g., no more than 0.2%.

The present inventors have determined that the catalysts described here can provide desirably high CO selectivity and desirably low methane selectivity at commercially relevant conversion rates. As used herein, a “conversion” is a molar fraction of a feed that is reacted (be it to desirable products or undesirable species). In various embodiments of the present disclosure as described herein, the reverse water-gas shift reaction has a CO₂ conversion of at least 5%, e.g., at least 10%, or 20%. For example, in some embodiments, the reverse water-gas shift reaction has a CO₂ conversion of at least 30%, e.g., at least 40%, or 50%, or 60%. In various embodiments of the present disclosure as described herein, the reverse water-gas shift reaction has a CO₂ conversion of no more than 90%, e.g., no more than 80% or no more than 70%. For example, in some embodiments, the reverse water-gas shift reaction has a CO₂ conversion of no more than 65%, e.g., no more than 60%. For example, in various embodiments as otherwise described herein, the CO₂ conversion is in the range of 10-90%, e.g., 10-80%, or 10-70%, or 10-60%, or 10-65%, or 20-90%, or 20-80%, or 20-70%, or 20-60%, or 20-65%, or 30-90%, or 30-80%, or 30-70%, or 30-60%, or 30-65%, or 40-90%, or 40-80%, or 40-70%, or 40-60%, or 40-65%. The person of ordinary skill in the art will, based on the disclosure herein, operate at a degree of conversion that provides a desirable product. And of course, in other embodiments, e.g., when in a stacked-bed or mixed-bed system, the CO₂ conversion may be even higher than described here.

Advantageously, the processes described herein can be performed at temperatures that are lower than temperatures used in many conventional reverse water-gas shift processes. As described above, various processes of the disclosure can be performed in a temperature range of 200-900° C. For example, in some embodiments, the method for performing the reverse water-gas shift reaction is conducted at a temperature in the range of 200-850° C., e.g., in the range of 200-800° C., or 200-750° C., or 200-700° C., or 200-650° C., or 200-600° C. In some embodiments, the method for performing the reverse water-gas shift reaction is conducted at a temperature in the range of 250-900° C., e.g., in the range of 250-850° C., or 250-800° C., or 250-700° C., or 250-650° C., or 250-600° C. In some embodiments of the present disclosure as described herein, the method for performing the reverse water-gas shift reaction is conducted at a temperature in the range of 300-900° C., e.g., in the range of 300-850° C., or 300-800° C., or 300-750° C., or 300-700° C., or 300-650° C., or 300-600° C. In some embodiments of the present disclosure as described herein, the method for performing the reverse water-gas shift reaction is conducted at a temperature in the range of 350-900° C., e.g., in the range of 350-850° C., or 350-800° C., or 350-750° C., or 350-700° C., or 350-650° C., or 350-600° C. In some embodiments, the method for performing the reverse water-gas shift reaction is conducted at a temperature in the range of 400-900° C., e.g., in the range of 400-850° C., or 400-800° C., or 400-750° C., or 400-700° C., or 400-650° C., or 400-600° C. In some embodiments, the method for performing the reverse water-gas shift reaction is conducted at a temperature in the range of 450-900° C., e.g., in the range of 450-850° C., or 450-800° C., or 450-750° C., or 450-700° C., or 450-650° C., or 450-600° C. In some embodiments, the method for performing the reverse water-gas shift reaction is conducted at a temperature in the range of 500-900° C., e.g., in the range of 500-850° C., or 500-800° C., or 500-750° C., or 500-700° C., or 500-650° C., or 500-600° C. In some embodiments, the method for performing the reverse water-gas shift reaction is conducted at a temperature in the range of 550-900° C., e.g., in the range of 550-850° C., or 550-800° C., or 550-750° C., or 550-700° C., or 550-650° C., or 550-600° C.

In some embodiments, the reverse water-gas shift reaction is conducted at a temperature in the range of 200-500° C., e.g., 200-450° C., or 200-400° C., or 200-350° C., or 250-500° C., or 250-450° C., or 250-400° C., or 250-350° C. The present inventors have noted that operation at these temperatures can provide for lower energy demand.

As described above, the feed stream includes CO₂ and H₂. Advantageously, the present inventors have recognized that both of these can come from renewable or otherwise environmentally responsible sources. For example, at least part of the H₂ can be so-called “green” hydrogen, e.g., produced from the electrolysis of water operated using renewable electricity (such as wind, solar, or hydroelectric power). In other embodiments, at least part of the H₂ may be from a so-called “blue” source, e.g., from a natural gas reforming process with carbon capture. Of course, other sources of hydrogen can be used in part or in full. For example, in some embodiments, at least a portion of the H₂ of the feed stream is grey hydrogen, black hydrogen, brown hydrogen, pink hydrogen, turquoise hydrogen, yellow hydrogen, and/or white hydrogen. CO₂ can be captured from the environment generally, or more directly from processes that form CO₂ (especially in difficult-to-abate sectors), making a product that is later made from the CO at least carbon-neutral. For example, in some embodiments, at least part of the CO₂ is from direct air capture, or from a manufacturing plant such as a bioethanol plant (e.g., CO₂ produced fermentation), a steel plant, or a cement plant. Accordingly, the rWGS reaction can be not only carbon neutral, but in some cases a net consumer of carbon dioxide. These benefits in particular makes the rWGS reaction highly attractive for decarbonizing transportation fuels, for both automotive and aviation sectors, since the carbon monoxide produced in the reaction can be readily utilized by well-established technologies to synthesize liquid hydrocarbon fuels.

The feed stream contains both H₂ and CO₂ (e.g., provided to a reaction zone in a single physical stream or multiple physical streams). As used herein, the feed stream includes all feeds to the process, regardless of whether provided as a mixture of gases or as gases provided individually to a reaction zone. In various embodiments as otherwise described herein, the molar ratio of H₂ to CO₂ in the feed stream is at least 0.1:1, e.g., at least 0.5:1. In some embodiments, the molar ratio of H₂ to CO₂ in the feed stream is at least 0.9:1, e.g., at 1:1 or least 1.5:1. In some embodiments, the molar ratio of H₂ to CO₂ in the feed stream is at least 2:1, e.g., at least 2.5:1. In some embodiments, the molar ratio of H₂ to CO₂ in the feed stream is no more than 100:1, e.g., no more than 75:1, or 50:1. In some embodiments, the molar ratio of H₂ to CO₂ in the feed stream is no more than 20:1, e.g., no more than 15:1, or 10:1. For example, in some embodiments, the molar ratio of H₂ to CO₂ in the feed stream is in the range of 0.5:1 to 10:1. The person of ordinary skill in the art will provide a desired ratio of H₂:CO₂ in the feed stream, based on the disclosure herein, that provides a desirable conversion and selectivity; excess H₂ can, if consistent with a desirable conversion and selectivity, be provided to flow through the system and provide a product stream with a desirable ratio of H₂ to CO for a downstream process.

Other gases may also be included in the feed stream. For example, in some embodiments, the feed stream further comprises CO. In some embodiments of the disclosure as otherwise described herein, the feed stream further comprises one or more inert gases. For example, in some embodiments, the feed stream further comprises nitrogen and/or methane.

The processes described herein can be performed at a variety of pressures, as would be appreciated by the person of ordinary skill in the art. In various embodiments of the present disclosure, the method for performing the reverse water-gas shift reaction is conducted at a pressure in the range of 1 to 100 barg. For example, the method is conducted at a pressure in the range of 1 to 70 barg, or 1 to 50 barg, or 1 to 40 barg, or 1 to 35 barg, or 5 to 70 barg, or 5 to 50 barg, or 5 to 40 barg, or 5 to 35 barg, or 10 to 70 barg, 10 to 50 barg, or 10 to 40 barg, or 10 to 35 barg, or 20 to 70 barg, 20 to 50 barg, or 20 to 40 barg, or 20 to 35 barg, or 25 to 70 barg, 25 to 50 barg, or 25 to 40 barg, or 25 to 35 barg.

The processes described herein can be performed at a variety of GHSV (gas hourly space velocity), as would be appreciated by the person of ordinary skill in the art. As such, the GHSV for performing the reverse water-gas shift reaction is not particularly limited. For example, in some embodiments of the present disclosure, the method for performing the reverse water-gas shift reaction is conducted at a GHSV in the range of 1,000 to 2,000,000 h⁻¹. In various embodiments, the method for performing the reverse water-gas shift reaction is conducted at a GHSV in the range of 1,000 to 1,200,000 h⁻¹, or 1,000 to 500,000 h⁻¹, or 1,000 to 100,000 h⁻¹, or 5,000 to 1,200,000 h⁻¹, or 5,000 to 500,000 h⁻¹, or 5,000 to 100,000 h⁻¹, or 10,000 to 1,200,000 h⁻¹, or 10,000 to 500,000 h⁻¹, or 10,000 to 100,000 h⁻¹. In various embodiments of the present disclose, the method for performing the reverse water-gas shift reaction is conducted at a GHSV in the range of 1,000 to 50,000 h⁻¹, or 2,000 to 50,000 h⁻¹, or 5,000 to 50,000 h⁻¹, or 10,000 to 50,000, or 1,000 to 40,000 h⁻¹, or 2,000 to 40,000 h⁻¹, or 5,000 to 40,000 h⁻¹, or 10,000 to 40,000 h⁻¹, or 1,000 to 30,000 h⁻¹, or 2,000 to 30,000 h⁻¹, or 5,000 to 30,000 h⁻¹, or 10,000 to 30,000 h⁻¹.

The rWGS catalyst described herein is based in part on platinum, palladium, and/or gold. It will typically be desirable to activate the rWGS catalyst, e.g., before contacting with the feed stream. Thus in some embodiments of the present disclosure as described herein, the method comprises activating the rWGS catalyst prior to contacting the catalyst with the feed stream. For example, in some embodiments, activating the catalyst comprises contacting the catalyst with a reducing stream comprising a reductive gas, e.g., hydrogen. In various embodiments of the present disclose, the reducing stream comprises hydrogen in an amount of at least 25 mol %, e.g., at least 50 mol %, or 75 mol %, or 90 mol %. The person of ordinary skill in the art will determine suitable conditions for activation of the rWGS catalyst. As such, the person or ordinary skill in the art would be able to choose an appropriate temperature, pressure, and time for activating the rWGS catalyst. For example, in various embodiments activating the catalyst is conducted at a temperature in the range of 200° C. to 800° C. In some embodiment, activating the catalyst is conducted at a temperature in the range of 250° C. to 800° C., or 300° C. to 800° C., or 200° C. to 700° C., or 250° C. to 800° C., or 300° C. to 700° C. In some embodiments of the present disclosure as described herein, activating the catalyst provides a catalyst that is at least 10% reduced (e.g., at least 25%, or at least 50% reduced).

The present inventors have found that contacting the rWGS catalysts as described herein with a feed stream can provide a product stream with advantageously high CO selectivity and low methane selectivity. The amount of CO in the product stream can be further controlled by the rWGS reaction conditions, as described above. But in general, the methods for performing the rWGS reaction, as described herein, provide a product stream comprising H₂ and CO, with the product stream having a lower concentration of CO₂ and a higher concentration of CO than the feed stream, as is consistent with the degrees of conversion described herein. For example, in various embodiments, the product stream includes no more than 95 mol % CO₂, or no more than 90 mol % CO₂. In some embodiments, the product stream includes no more than 85 mol % CO₂, or no more than 80 mol % CO₂. In other examples, the product stream includes no more than 75 mol %, or no more than 70 mol % CO₂. However, as described above, the present inventors have determined that it can be desirable to perform the processes at intermediate degrees of conversion to provide desirably high CO selectivities and desirably low methane selectivities. Accordingly, in various embodiments as otherwise described herein, the product stream includes an amount of CO₂ together with the CO.

Other gases may also be included in the product stream. In some embodiments of the disclosure as otherwise described herein, the product stream further comprises one or more inert gases. These inert gases may be included from the feed stream or provided from a source other than the feed stream. For example, in some embodiments, the product stream further comprises nitrogen and/or methane.

Depending on, inter alia, the degree of conversion, the CO selectivity, the relative amounts of H₂ and CO₂ in the feed stream, and the reaction conditions, the product stream can include H₂ in combination with CO, in a variety of ratios. For example, in some embodiments, the ratio of H₂:CO in the product stream is in the range of 0.1:1 to 100:1 (e.g., in the range of 0.1:1 to 50:1, or 0.1:1 to 25:1, or 0.1:1 to 10:1, or 0.1:1 to 5:1, or 1:1 to 100:1, or 1:1 to 50:1, or 1:1 to 25:1, or 1:1 to 10:1, or 1:1 to 5:1).

The person of ordinary skill in the art would appreciate that, based on the methods as described herein, the product stream may include H₂, CO, and CO₂ and other components in various amounts. Components of the product stream may be separated and used for various purposes in the rWGS process.

For example, in various embodiments of the present disclosure as described herein, the method further comprises separating the product stream to recycle at least a portion (e.g., at least 5 mol %, at least 10 mol %, at least 25 mol %, at least 50 mol %, at least 75 mol %, or at least 90 mol %) of one or more components of the product stream to the feed stream. For example, when the product stream includes CO₂, the method can include recycling at least a portion (e.g., at least 5 mol %, at least 10 mol %, at least 25 mol %, at least 50 mol %. at least 75 mol %, or at least 90 mol %) of the CO₂ of the product stream to the feed stream. The product stream may also include H₂; in some embodiments, the method further includes recycling at least a portion of H₂ of the product stream (e.g., at least 5 mol %, at least 10 mol %, at least 25 mol %, at least 50 mol %, at least 75 mol %, or at least 90 mol %) to the feed stream.

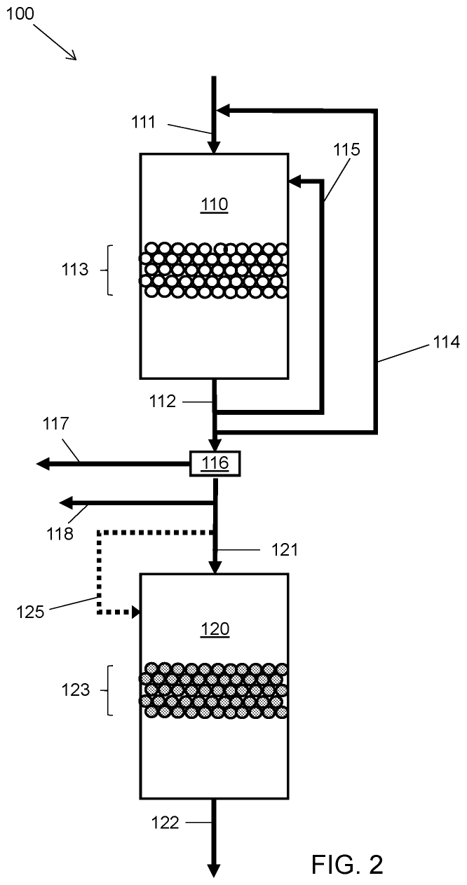

Such recycling is shown in the process 100 of FIG. 1 (and of FIG. 2). Here, the process 100 includes separating from the product stream 112 at least a portion of CO₂ (stream 114) to recycle to the feed stream 111. Similarly, the process 100 includes separating from the product stream 112 at least a portion of H₂ (stream 115) to recycle to the product stream 111. While stream 115 is depicted as entering reactor 110 through a different inlet than the rest of the feed stream 111, it is considered to be part of the feed stream, as it is part of the material input to the process step.

As noted above, one competing reaction in the reverse water-gas shift reaction is the Sabatier reaction, which makes methane. While in various embodiments, the reverse water-gas shift processes described herein can be performed without forming large amounts of methane, in some embodiments there can be some methane formed. Accordingly, in various embodiments of the method as described herein, the product stream comprises one or more light hydrocarbons. For example, in some embodiments, the product stream may include one or more of methane, ethane, propane, or combinations thereof. As would be understood by the person of ordinary skill in the art, it may be desirable to operate the reverse water-gas shift reaction to provide higher amounts of light hydrocarbons in the product feed. For example, such light hydrocarbons may be inert in further processing of the product stream and so may be acceptable at higher amounts. The person of ordinary skill in the art would be able to select appropriate reaction conditions (e.g., temperature, pressure, feed stream composition) to provide a product stream that includes methane at a desired amount. For example, in various embodiments as otherwise described herein, the product stream includes no more than 20 mol % methane or no more than 15 mol %. As noted above, when lower amounts of methane are desired in the product stream, the catalysts of the disclosure can provide very low methane selectivity. Accordingly, in various embodiments as otherwise described herein, the product stream includes no more than 10 mol % methane. For example, in various embodiments, the product stream includes no more than 5 mol %, or 1 mol %, or 0.5 mol %, or no more than 0.1 mol % methane. In general, light hydrocarbons (e.g., C₁-C₅ hydrocarbons) may be present in the product stream. For example, in various embodiments as otherwise described herein, the product stream includes no more than 20 mol % light hydrocarbons (e.g., no more than 15 mol %, no more than 10 mol %, no more than 5 mol %, no more than 1 mol %, no more than 0.5 mol %, or no more than 0.1 no mol % light hydrocarbons).

These light hydrocarbons (e.g., C₁-C₅ hydrocarbons) of the product stream can be separated and used for other purposes. For example, in various embodiments, the method further includes separating at least a portion of one or more light hydrocarbons from the product stream to provide a light hydrocarbon stream. For example, in method 100 of FIG. 1, at least a portion of one or more light hydrocarbons are separated from the product stream 112 to provide a light hydrocarbon stream 116. The light hydrocarbon stream, for example, can be used to provide other products, can be partially oxidized to form CO, can be steam reformed to provide hydrogen, and/or can be burned to provide heat or other energy (e.g., electricity for electrolysis) for use in the rWGS method or otherwise. Of course, as would be understood by the ordinary skill in the art, the light hydrocarbon stream may be used in other processes as well. For example, as would be understood by the person of ordinary skill in the art, some rWGS catalysts can have reforming capability. Without being bound by theory, the present inventors hypothesize that one explanation for the low methane production observed using the rWGS catalysts as described herein is that methane is formed but then immediately reformed into CO and H₂. Accordingly, in some embodiments as described herein, the light hydrocarbons of the process stream is recycled to the feed stream for the rWGS reaction. These light hydrocarbons can be separated and used for other purposes. For example, in various embodiments, the process further includes separating at least a portion of one or more light hydrocarbons from the first product stream to provide a light hydrocarbon stream. For example, in process 100 of FIG. 2, at least a portion of one or more light hydrocarbons are separated from the first product stream 112 to provide a light hydrocarbon stream 118. The light hydrocarbon stream, for example, can be used to provide other products, can be partially oxidized to form CO, can be steam reformed to provide hydrogen, and/or can be burned to provide heat or other energy (e.g., electricity for electrolysis) for use in the integrated process or otherwise. Of course, as would be understood by the person of ordinary skill in the art, the light hydrocarbon stream may be used in other processes as well.

### Integrated Fischer-Tropsch Processes

In some embodiments as described herein, the supported reverse water-gas can be used in a rWGS process integrated with an FT process. The embodiments related to the reverse water-gas shift portion of the integrated FT process are as described above in the previous section. For example, the feed stream of the rWGS process corresponds to the first feed stream in the integrated FT process and the product stream of the rWGS process corresponds to the first product stream in the integrated FT process.

Another aspect of the present disclosure provides a process for performing an integrated Fischer-Tropsch process (i.e., integrated with an rWGS process). The process includes providing a first feed stream comprising H₂ and CO₂; contacting at a first temperature in the range of 200-900° C. and at a first pressure a reverse water-gas shift catalyst, as described herein, with the first feed stream to perform a reverse water-gas shift reaction to provide a first product stream comprising CO and H₂, the first product stream having a lower concentration of CO₂ and a higher concentration of CO than the first stream feed stream. An example of such a process is shown schematically in FIG. 2. In FIG. 2, the process 100 includes performing a reverse water-gas shift reaction by providing a first feed stream 111 comprising H₂ and CO₂, here, to a first reaction zone, e.g., a reactor 110. A reverse water-gas shift catalyst 113, as described herein, is contacted at a first temperature in the range of 200-900° C. and at a first pressure with the feed stream 111 to provide a first product stream 112 comprising CO and H₂. The first product stream has a lower concentration of CO₂ and a higher concentration of CO than the first feed stream. The process of this aspect of the disclosure also includes contacting at a second temperature and at a second pressure a Fischer-Tropsch catalyst with a second feed stream comprising H₂ and at least a portion of CO of the first product stream to provide a second product stream comprising C₅₊ hydrocarbons, In the process 100 of FIG. 2, at least a portion of CO of the first product stream 112 is included in second feed stream 121, which is contacted with Fischer-Tropsch catalyst 123, here, in a second reaction zone (e.g., a reactor 120). This provides a second product stream 122, which includes C₅₊ hydrocarbons.

The present inventors have found that contacting the rWGS catalysts as described herein with a first feed stream can provide a first product stream with advantageously high CO selectivity and low methane selectivity. The amount of CO in the first product stream can be further controlled by the rWGS reaction conditions, as described above.

However, as described above, the present inventors have determined that it can be desirable to perform the processes at intermediate degrees of conversion to provide desirably high CO selectivities and desirably low methane selectivities. Moreover, the present inventors have noted that it can be advantageous to perform the downstream Fischer-Tropsch process with a relatively high level of inerts, and thus contemplate that passing a significant amount of CO₂ to the Fischer-Tropsch process step can be beneficial. Accordingly, in various embodiments as otherwise described herein, the first product stream includes an amount of CO₂ together with the CO. In various embodiments, the first product stream comprises in the range of 5-95 mol % CO₂, e.g., 5-90 mol %, or 5-85 mol %, or 5-80 mol %, or 5-75 mol %, or 5-70 mol %, or 10-95 mol %, or 10-90 mol %, or 10-85 mol %, or 10-80 mol %, or 10-75 mol %, or 10-70 mol %, or 20-95 mol %, or 20-90 mol %, or 20-85 mol %, or 20-80 mol %, or 20-75 mol %, or 20-70 mol %, or 30-95 mol %, or 30-90 mol %, or 30-85 mol %, or 30-80 mol %, or 30-75 mol %, or 30-70 mol % CO₂.

Moreover, as described below, Fischer-Tropsch catalysts typically require activation by a reducing gas. As would be understood by the person of skill in the art, different Fischer-Tropsch catalysts required different activation conditions (e.g., gas composition, temperature, pressure, time). For example, iron based Fischer-Tropsch catalysts require activation with both H₂ and CO, while cobalt based Fischer-Tropsch catalysts require activation with just H₂. As such, H₂ and CO or just H₂ from the first product stream can be used to perform this activation. Accordingly, in various embodiments as otherwise described herein, the process includes separating at least a portion of H₂ and CO (desirably in a ratio of at least 1:1 or at least 3:1) from the first product stream and contacting it with the Fischer-Tropsch catalyst to activate the Fischer-Tropsch catalyst. In various other embodiments as otherwise described herein, the process includes separating at least a portion of H₂ from the first product stream and contacting it with the Fischer-Tropsch catalyst to activate the Fischer-Tropsch catalyst. For example, in the process of FIG. 2, stream 125 separates H₂ or H₂ and CO and conducts it to reactor 120. This separation need not be continuous; rather, it need only be performed for a time desirable to provide reducing gas to the Fischer-Tropsch catalyst for activation. Of course, as would be understood by the person of skill in the art, other sources of H₂ or CO may be used to provide reducing gas to the Fischer-Tropsch catalyst for activation.

As shown above, water is a product of the reverse water-gas shift reaction. Accordingly, the first product stream will generally contain water. In many cases, it can be desirable to reduce the amount of water that is provided to the Fischer-Tropsch process step. Accordingly, in various embodiments as otherwise described herein, the process further includes removing at least a portion (e.g., at least 25%, at least 50%, or at least 75%) of water from the first product stream. In the embodiment of FIG. 2, a water removal zone 116 is used to remove water and provide a water-containing stream 117. The person of ordinary skill in the art will appreciate that a variety of processes can be used to remove water from the first product stream. For example, the first product stream can be contacted with a water scavenger to remove water therefrom. For example, a molecular sieve guard bed can be used to remove water from the first product stream; water can be recovered from the molecular sieves of the guard bed, e.g., by heating and vacuum. In other embodiments, a knockout vessel can be used. However, use of a knockout vessel can in some cases cool the first product stream enough so that it is desirably reheated for introduction to the Fischer-Tropsch process step. Water removed from the first product stream can be used, e.g., as feed water for electrolysis processes described herein.

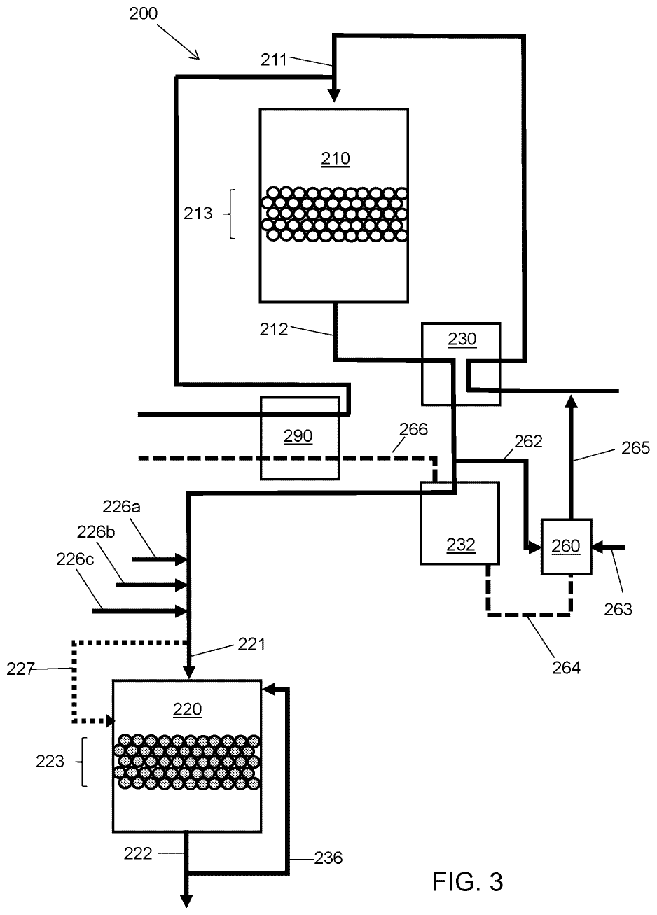

As noted above, the reverse water-gas shift process can be provided at a wide variety of temperatures. In some cases, those temperatures can be relatively close to the temperature of the subsequent Fischer-Tropsch process step (often 150-400° C., e.g., 200-350° C., or other temperatures as described below). In other cases, the reverse water-gas shift process can be performed at temperatures significantly higher than the temperature of the Fischer-Tropsch step. The present inventors have noted that it can be desirable to provide for heat exchange with a relatively hot first product stream to cool the first product stream to a temperature more appropriate for the Fischer Tropsch step and to provide heat elsewhere to the integrated process. For example, in various embodiments of the processes as otherwise described herein, the process further comprises exchanging heat between at least a portion of the first product stream and at least a portion of the first feed stream, thereby cooling at least a portion of the first product stream and heating at least a portion of the first feed stream. An example of such a process is shown schematically in FIG. 3. In FIG. 3, the process 200, first reactor 210, first feed stream 211, first product stream 212, reverse water-gas shift catalyst 213, second reactor 220, second feed stream 221, second product stream 222 and Fischer-Tropsch catalyst 223 are generally as described above. Here, the process 200 includes exchanging heat between at least a portion of the first product stream 212 and a least a portion of the first feed stream 211 in a first heat exchange zone 230, thereby cooling at least a portion of the first product stream 212 and heating at least a portion of the first feed stream 211. The person of ordinary skill in the art will appreciate that a wide variety of heat exchangers can be used for this purpose.

Of course, any excess heat in the first product stream can be additionally or alternatively used for other purposes. For example, in various embodiments the process further comprises exchanging heat between at least a portion of the first product stream and a steam generation zone, thereby cooling at least a portion of the first product stream and providing heat to the steam generation zone. This is shown in FIG. 3. Here, after heat exchange with the first feed stream 211, the first product stream 212 is conducted to steam generation zone 232, to cool the first product stream 212 and provide heat to the steam generation zone 232. Steam can be generated from the heat provided, and electricity can be generated from the steam. For example, in the embodiment of FIG. 3, electricity stream 264 is provided by the generation of electricity using steam generated in the steam generation zone 232. Of course, as would be understood to the person of ordinary skill in the art, the steam generated in the steam generation zone may be used in other processes. In various embodiments, the steam may be used to heat the first feed stream. For example, in the embodiment of FIG. 3, the steam stream 266 generated in the steam generation zone 232 is conducted to the heat exchange zone 290 to heat the first feed stream 211.

As noted above, at least a portion of the CO of the first product stream is included in the second feed stream, for reaction in a Fischer-Tropsch process. For example, in various embodiments as otherwise described herein, at least 25% of the CO of the first product stream, e.g., at least 50% of the CO, at least 75% of the CO, or at least 90% of the CO of the first product stream is included in the second feed stream. Of course, as noted above, some of the CO of the first product stream can be used for other purposes, e.g., catalyst activation as described herein.

In some embodiments, substantially all of the CO of the second feed stream comes from the first product stream. However, in other embodiments, CO can be provided to the second feed stream from other sources, fossil-derived or otherwise. For example, in various embodiments, CO is provided to the second feed stream from a CO source other than the first product stream. In FIG. 3, a stream of CO 226a from some other source is included in the second feed stream 221. The person of ordinary skill in the art will appreciate that CO can be provided from a variety of sources, e.g., gasification, reforming, or electrochemical CO₂ reduction. Moreover, as described in more detail below, CO can be recycled to the second feed stream from the second product stream; and/or can be provided by reaction of a light hydrocarbon stream, e.g., by partial oxidation or reforming (e.g., steam reforming and/or autothermal reforming).

As noted above, the second feed stream includes H₂. Notably, the first product stream will often include H₂, e.g., unreacted from the first feed stream. In various embodiments, the first product stream includes H₂ wherein the second feed stream includes at least a portion of the H₂ of the first product stream. For example, in various embodiments as otherwise described herein, at least 25% of the H₂ of the first product stream, e.g., at least 50% of the H₂, at least 75% of the H₂, or at least 90% of the H₂ of the first product stream is included in the second feed stream. Of course, as noted above, some of the H₂ of the first product stream can be used for other purposes, e.g., catalyst activation as described herein.

In some embodiments, substantially all of the H₂ of the second feed stream comes from the first product stream. In fact, the person of ordinary skill in the art can provide more H₂ than necessary for the reverse water-gas shift reaction in the first feed stream, to provide excess H₂ in the first product stream that can then provide a desired amount of H₂ to the second feed stream for the Fischer-Tropsch process step. However, in other embodiments, H₂ can be provided to the second feed stream from other sources. For example, in various embodiments, H₂ is provided to the second feed stream from a H₂ source other than the first product stream. In FIG. 3, a stream of H₂ 226b from some other source is included in the second feed stream 221. The person of ordinary skill in the art will appreciate that H₂ can be provided from a variety of sources, e.g., gasification, reforming, or H₂O electrolysis (including the electrolysis described herein). Moreover, as described in more detail below, H₂ can be recycled to the second feed stream from the second product stream.

The person of ordinary skill in the art can, based on the disclosure herein, adjust the relative amounts of H₂ and CO in the second feed stream to provide a desired ratio. For example, more or less H₂ of the first feed stream and/or more or less H₂ from electrolysis can be included in the second feed stream. Similarly, more or less CO of the first feed stream and more or less CO from other sources (e.g., partial oxidation and reforming as described below) can be included in the second feed stream.

As noted above, it can be desirable to perform the Fischer-Tropsch process step in the presence of a significant level of inerts. One such inert, CO₂, can come from the reverse water-gas shift, e.g., via the first product stream. Accordingly, in various embodiments as otherwise described herein, the second feed stream includes at least a portion of CO₂ of the first product stream. For example, in various embodiments, at least 10% of the CO₂ of the first product stream, e.g., at least 25% of the CO₂, at least 50% of the CO₂, at least 75% of the CO₂, or at least 90% of the CO₂ of the first product stream is included in the second feed stream. Of course, in other embodiments, the second feed stream may not include any substantial amount of CO₂ of the first product stream. Accordingly, in various embodiments, the second feed stream does not include a substantial amount of CO₂ of the first product stream. While it can be desirable generally to recycle CO₂ to the first feed stream for use in the reverse water-gas shift reaction, as described in more detail below, unreacted CO₂ can be recycled from the second product stream to the first feed stream.

But it can additionally or alternatively be desirable to include additional inert content to the second feed stream, be it CO₂ or other inerts such as nitrogen and methane. For example, in various embodiments, one or more inerts (e.g., CO₂, nitrogen and/or methane) are provided to the second feed stream from a source other than the first product stream. In FIG. 3, a stream of inert(s) 226c from some other source is included in the second feed stream 221. The person of ordinary skill in the art will appreciate that inerts can be provided from a variety of sources. Moreover, as described in more detail below, inerts can be recycled to the second feed stream from the second product stream.

As noted above, it can be desirable to perform the Fischer-Tropsch process step in the presence of inerts. Accordingly, in various embodiments as otherwise described herein, the portion of the first product stream that is included in the second feed stream has a CO₂ content in the range of 10-95 mol % CO₂, e.g., 10-90 mol %, or 10-85 mol %, or 10-80 mol %, or 10-75 mol %, or 10-70 mol %, or 20-95 mol %, or 20-90 mol %, or 20-85 mol %, or 20-80 mol %, or 20-75 mol %, or 20-70 mol %, or 30-95 mol %, or 30-90 mol %, or 30-85 mol %, or 30-80 mol %, or 30-75 mol %, or 30-70 mol % CO₂.

Other gases may also be included in the second feed stream, as described above. For example, as noted above, it can be desirable to perform the Fischer-Tropsch process step in the presence of a significant amount of inerts (i.e., components that are not H₂ or CO). For example, in various embodiments, the second feed stream includes up to 80 mol % of one or more inerts, e.g., in the range of 3-80 mol %, or 5-80 mol %, or 10-80 mol %, or 15-80 mol %, or 30-80 mol % of one or more inerts. In various embodiments, the second feed stream includes up to 70 mol % inerts, up to 60 mol % inerts, or up to 50 mol % inerts, e.g., 3-70 mol %, or 5-70 mol %, or 10-70 mol %, or 15-70 mol %, or 30-70 mol %, or 3-60 mol %, or 5-60 mol %, or 10-60 mol %, or 15-60 mol %, or 30-60 mol %, or 3-50 mol %, or 5-50 mol %, or 10-50 mol %, or 15-50 mol %, or 30-50 mol % inerts. In various embodiments, the second feed stream includes up to 80% of one or more inerts selected from CO₂, methane and nitrogen, e.g., up to 70 mol %, up to 60 mol %, or up to 50 mol %, or 15-70 mol %, or 30-70 mol %, or 15-60 mol %, or 30-60 mol %, or 15-50 mol %, or 30-50 mol %. In various embodiments, the second feed stream includes up to 80 mol % of CO₂, e.g., up to 70 mol %, up to 60 mol %, or up to 50 mol %, or 15-70 mol %, or 30-70 mol %, or 15-60 mol %, or 30-60 mol %, or 15-50 mol %, or 30-50 mol %.

The person of ordinary skill in the art can tune the portion of the first product stream that is included in the second feed stream to provide a desirable H₂:CO ratio. For example in various embodiments, the portion of the first product stream that is included in the second feed stream has a H₂:CO ratio in the range of 0.5:1 to 10:1, e.g., in the range of 1:1 to 2.5:1. Of course, whatever the H₂:CO ratio of the portion of the first product stream that is included in the second feed stream, the person of ordinary skill in the art can add H₂ or CO as described above as necessary to provide the desired ratio overall in the second feed stream.

As described above, the second feed stream contains both H₂ and CO and the second feed stream includes all feeds to the Fischer Tropsch reactor, regardless of whether the second feed stream is provided as a mixture of feeds or as feeds provided individually to a reaction zone. In various embodiments of the present disclosure as described herein, the second feed stream has a H₂:CO ratio in the range of 0.5:1 to 6:1. In some embodiments, the second feed stream has a H₂:CO ratio in the range of 1:1 to 3:1, or 1:1 to 2.5:1. In some embodiments, the second feed stream has a H₂:CO ratio of at least 1.4:1. For example, in some embodiments, the second feed stream has a H₂:CO ratio in the range of 1.4:1 to 3:1, or 1.4:1 to 2:1. The person of ordinary skill in the art will provide a desired ratio of H₂:CO in the second feed stream, based on the disclosure herein that provides a desirable conversion and selectivity in the Fischer-Tropsch process.

As noted above it can be desirable to reduce the amount of water that is conducted to the Fischer-Tropsch process step. Accordingly, in various embodiments as otherwise described herein, the portion of the first product stream that is included in the second feed stream has a water content of no more than 10 mol %, e.g., or no more than 2 mol %, or no more than 0.5 mol %.

And as noted above, it can be desirable to perform the Fischer-Tropsch process with a relatively small amount of water present. Accordingly, in various embodiments, the second feed stream has a water content of no more than 10 mol %, e.g., or no more than 2 mol %, or no more than 0.5 mol %.

The processes as described here include contacting a Fischer-Tropsch catalyst with the second feed stream as described herein. The Fischer-Tropsch catalyst for use in the processes as described herein is not particularly limited and the person of ordinary skill in the art would be able to choose a catalyst as appropriate for their desired Fischer-Tropsch product. In some embodiments, the Fischer-Tropsch catalyst includes cobalt, iron, rhodium, ruthenium, or a combination thereof.

For example, in some embodiments of the present disclosure as described herein, the Fischer-Tropsch catalyst comprises cobalt, e.g., in an amount in the range of 5-25 wt %, calculated as Co(0). “Calculated as Co(0)” and analogous terms mean that the weight of cobalt atoms/ions themselves are used in the calculation, and not the total amount of any compound or polynuclear ion in which those cobalt atoms/ions might be bound. For example, in various embodiments, the Fischer-Tropsch catalyst comprises cobalt in an amount in the range of 7-25 wt %, or 10-25 wt %, or 5-20 wt %, or 7-20 wt %, or 10-20 wt %, calculated as Co(0). As the person of ordinary skill in the art will appreciate, cobalt-based catalysts are often provided to the reaction zone in the form of cobalt oxide on a support; the cobalt can be reductively activated (e.g., with H₂) in situ to provide an active catalyst species with a significant concentration of Co(0).

In some embodiments, the Fischer-Tropsch catalyst comprises iron, e.g., in an amount in the range of 5-95 wt %, calculated as Fe(0). For example, in various embodiments, the Fischer-Tropsch catalyst comprises iron in the range of 10-95 wt %, or 25-95 wt %, or 50-95 wt %, or 5-85 wt %, or 10-85 wt %, or 25-85 wt %, or 50-85 wt %, or 5-75 wt %, or 10-75 wt %, or 25-75 wt %, calculated as Fe(0). As the person of ordinary skill in the art will appreciate, iron-based catalysts are often provided to the reaction zone in the form of metallic iron or iron oxide, optionally on a support; the iron can be activated (e.g., by reaction with H₂ and CO) to provide an active catalyst species with a significant concentration of iron carbide.

In various embodiments of the disclosure as described herein, especially when the catalyst is a cobalt-based catalyst, the Fischer-Tropsch catalyst further includes manganese. For example, in various embodiments, the Fischer-Tropsch catalyst includes manganese in an amount up to 15 wt %, e.g., up to 12 wt %, or up to 10 wt %, or up to 7 wt %, calculated as Mn(0). In certain such embodiments, a catalyst material includes manganese in an amount in the range of 0.1-15 wt %, e.g., 0.1-10 wt %, or 0.1-5 wt %, 0.5-15 wt %, or 0.5-10 wt %, or 0.5-5 wt %, or calculated as Mn(0). Of course, in other embodiments substantially no manganese is present (e.g., less than 0.1 wt % or less than 0.5 wt % manganese is present).

The Fischer-Tropsch catalysts suitable for use in the process as described herein can be a variety of forms and are not particularly limited. For example, the Fischer-Tropsch catalyst may be a supported or unsupported catalyst. While the form of the catalyst is not particularly limited, in various desirable embodiments, the Fischer-Tropsch catalyst is a supported catalyst, wherein the support comprises at least one of titanium oxide, zirconium oxide, cerium oxide, aluminum oxide, silicon oxide and zinc oxide. For example, in various embodiments, the support comprises at least one or titanium oxide, aluminum oxide, and silicon oxide. In some embodiments of the present disclosure as described herein, the support is a titanium dioxide support.

The person of ordinary skill in the art will appreciate that the Fischer-Tropsch catalysts of the disclosure can be provided in many forms, depending especially on the particular form of the reactor system in which they are to be used, e.g., in a fixed bed or as a fluidized bed. The supports of the Fischer-Tropsch catalysts can be provided themselves as discrete bodies of material, e.g., as porous particles, pellets or shaped extrudates, with the metals provided thereon to provide the Fischer-Tropsch catalyst. However, in other embodiments, a Fischer-Tropsch catalyst of the disclosure can itself be formed as a layer on an underlying substrate. The underlying substrate is not particularly limited. It can be formed of, e.g., a metal or metal oxide, and can itself be provided in a number of forms, such as particles, pellets, shaped extrudates, or monoliths. The person of ordinary skill in the art will select an appropriate Fischer-Tropsch catalyst for the particular reactor system.

As with the rWGS catalyst, Fischer-Tropsch catalysts are typically activated before use, e.g., to provide cobalt(0) species on a cobalt-based catalyst, or iron carbide species on an iron-based catalyst. Such activation can be performed prior to contacting the Fischer-Tropsch catalyst with a second feed stream.

For example, in some embodiments, the Fischer-Tropsch catalyst is activated by contact with a reducing gas. For example, hydrogen can be an especially suitable gas for activating Fischer-Tropsch catalyst, e.g., when the activation is a reduction to metal(0) species, e.g., as for many cobalt-based catalysts. In various embodiments of the present disclosure as otherwise described herein, the reducing gas comprises at least a portion of H₂ from the first product stream. For example, in some embodiments, the process further comprises separating at least a portion of H₂ of the first product stream and contacting it with the Fischer-Tropsch catalyst to activate Fischer-Tropsch catalyst. In the process 100 shown schematically in FIG. 2, at least a portion of hydrogen stream 125 is separated from the first product stream 112 and contacted with the Fischer-Tropsch catalyst 123 to activate it. In other embodiments, H₂ present in the second feed stream can be used to activate the catalyst. As would be understood by the person of ordinary skill in the art, activation temperatures can vary depending on the Fischer-Tropsch catalyst used. As such, the person of ordinary skill in the art would be able to select an appropriate temperature for activating the catalyst, e.g., in the range of 200-400° C.

In various embodiments, the Fischer-Tropsch catalyst is activated by contact with H₂ and CO. This can be especially suitable when the activation provides conversion to carbide, e.g., as for many iron-based catalysts. In various embodiments of the present disclosure as otherwise described herein, the reducing gas comprises at least a portion of H₂ and CO from the first product stream. For example, in some embodiments, the process further comprises separating at least a portion of H₂ and at least a portion of CO of the first product stream and contacting it with the Fischer-Tropsch catalyst to activate Fischer-Tropsch catalyst. In the process 200 shown schematically in FIG. 3, at least a portion of H₂ and CO stream 227 is separated from the first product stream 212 and contacted with the Fischer-Tropsch catalyst 223 to activate it. In other embodiments, H₂ and CO present in the second feed stream can be used to activate the catalyst. Activation temperatures can vary, e.g., in the range of 200-400° C.

As described above, the process includes contacting at a second temperature and at a second pressure a Fischer-Tropsch catalyst with a second feed stream. The person of ordinary skill in the art will select appropriate reaction conditions in conjunction with the particular feed and catalyst used to provide desired Fischer-Tropsch processes. In some embodiments of the disclosure as described herein, the second temperature is in the range of 150-400° C. For example, in various embodiments, the second temperature is in the range of 150-350° C., or 150-300° C., or 150-250° C., or 150-200° C., or 200-400° C., or 200-350° C., or 200-300° C., or 200-250° C., or 250-400° C., or 250-350° C., or 250-300° C., or 300-400° C. In some particular embodiments, the second temperature is in the range of 200-350° C.

Notably, in many embodiments, the first temperature and the second temperature can be relatively close to one another. The present inventors have noted that the reverse water-gas shift catalysts described herein can provide suitable activity and CO selectivity even at relatively low temperatures. Accordingly, the first product stream can be provided with a temperature that is suitable for, or at least close to suitable for, the Fischer-Tropsch reaction step. This can desirably provide for increased process integration. For example, in various embodiments, the first temperature is within 100° C. of the second temperature, e.g., within 50° C. of the second temperature, or within 25° C. of the second temperature.

However, in other embodiments, the first temperature and the second temperature are less close to one another. The present inventors have noted that in many cases a desirable reverse water-gas shift process temperature will be significantly greater than a desirable Fischer-Tropsch process temperature. For example, in various embodiments, the first temperature is at least 100° C. greater than the second temperature, e.g., at least 150° C. greater than the second temperature, or at least 200° C. greater than the second temperature. The excess heat in the first product stream can be used for a number of purposes, for example, to preheat at least part of the first feed stream or to generate steam for use in generating electricity, as described above.

In some embodiments of the disclosure as described herein, the second pressure is in the range of 10-50 barg. For example, in various embodiments, the second pressure is in the range of 20-50 barg, or 25-50 barg, or 10-40 barg, or 20-40 barg, or 25-40 barg or 10-35 barg, or 20-35 barg, or 25-35 barg. In some embodiments, the second pressure is in the range of 20-50 barg.

The Fischer-Tropsch processes described herein can be performed at a variety of GHSV (gas hourly space velocity) values, as would be appreciated by the person of ordinary skill in the art. As such, the GHSV for performing the Fischer-Tropsch reaction is not particularly limited. For example, in some embodiments of the present disclosure, the process for performing the Fischer-Tropsch reaction is conducted at a GHSV in the range of 1,000 to 2,000,000 h⁻¹. In various embodiments, the process for performing the reverse water-gas shift reaction is conducted at a GHSV in the range of 1,000 to 1,200,000 h⁻¹, or 1,000 to 500,000 h⁻¹, or 1,000 to 100,000 h⁻¹, or 5,000 to 1,200,000 h⁻¹, or 5,000 to 500,000 h⁻¹, or 5,000 to 100,000 h⁻¹, or 10,000 to 1,200,000 h⁻¹, or 10,000 to 500,000 h⁻¹, or 10,000 to 100,000 h⁻¹. In various embodiments of the present disclose, the process for performing the Fischer-Tropsch reaction is conducted at a GHSV in the range of 1,000 to 50,000 h⁻¹, or 2,000 to 50,000 h⁻¹, or 5,000 to 50,000 h⁻¹, or 10,000 to 50,000, or 1,000 to 40,000 h⁻¹, or 2,000 to 40,000 h⁻¹, or 5,000 to 40,000 h⁻¹, or 10,000 to 40,000 h⁻¹, or 1,000 to 30,000 h⁻¹, or 2,000 to 30,000 h⁻¹, or 5,000 to 30,000 h⁻¹, or 10,000 to 30,000 h⁻¹.

The Fischer-Tropsch process is typically used to make C₅₊ hydrocarbons, for example, unsubstituted C₅₊ hydrocarbons (e.g., alkanes and alkenes) and oxygenated C₅₊ hydrocarbons (e.g., C₅₊ alcohols, aldehydes, ketones, carboxylic acids). In various embodiments of the disclosure as described herein, contacting the Fischer-Tropsch catalyst with the second feed stream to provide the second product stream is performed with a C₅₊ selectivity (i.e., for all C₅₊ species) of at least 30%, e.g., at least 50%, or at least 70%. For example, in some embodiments, the selectivity for C₅₊ alkanes is at least 30%, e.g., at least 50%, or at least 70%. In some embodiments, the selectivity for C₅₊ alkanes and C₅₊ alcohols is at least 30%, e.g., at least 50%, or at least 70%.

Additional components may be in present in the second product stream. For example, in some embodiments, the second product stream includes water, which is another product of the Fischer-Tropsch reaction. Also present can be one or more light hydrocarbons (i.e., C₁-C₄) as a side product. CO and/or H₂ can be present, e.g., unreacted from the second feed stream. CO₂ or other inerts as described herein can also be present. Such components of the second product stream can be separated and/or recycled in various manners.

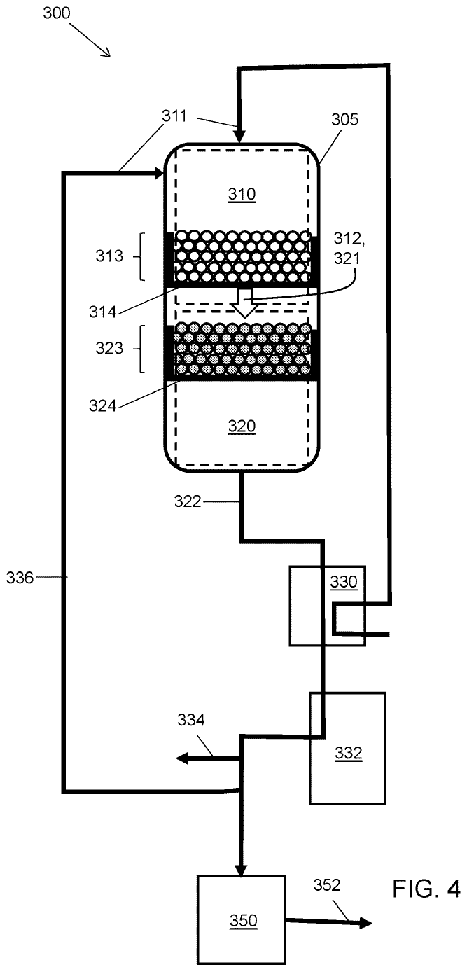

For example, in various embodiments, the process further comprises separating at least a portion of water from the second product stream. This is shown schematically in FIG. 4. In the embodiment of FIG. 4, the reverse water-gas shift catalyst 313 and the Fischer-Tropsch catalyst 323 are provided in separate beds in the same reactor. Thus, the first reaction zone 310 is a volume of the reactor 305 that includes the bed 314 containing the reverse water-gas shift catalyst 313, and the second reaction zone 320 is a volume of the reactor 305 that includes the bed 324 containing the Fischer-Tropsch catalyst 323. First feed stream 311 is contacted with the reverse water-gas shift catalyst 313 to provide first product stream 312, which is passed directly as the second feed stream 321 to the Fischer-Tropsch catalyst 323 to provide second product stream 322. Here, the process also optionally includes separating at least a portion of water (e.g., at least 50%, at least 75%, or at least 90%) from the second product stream 322 to provide water-containing stream 334.

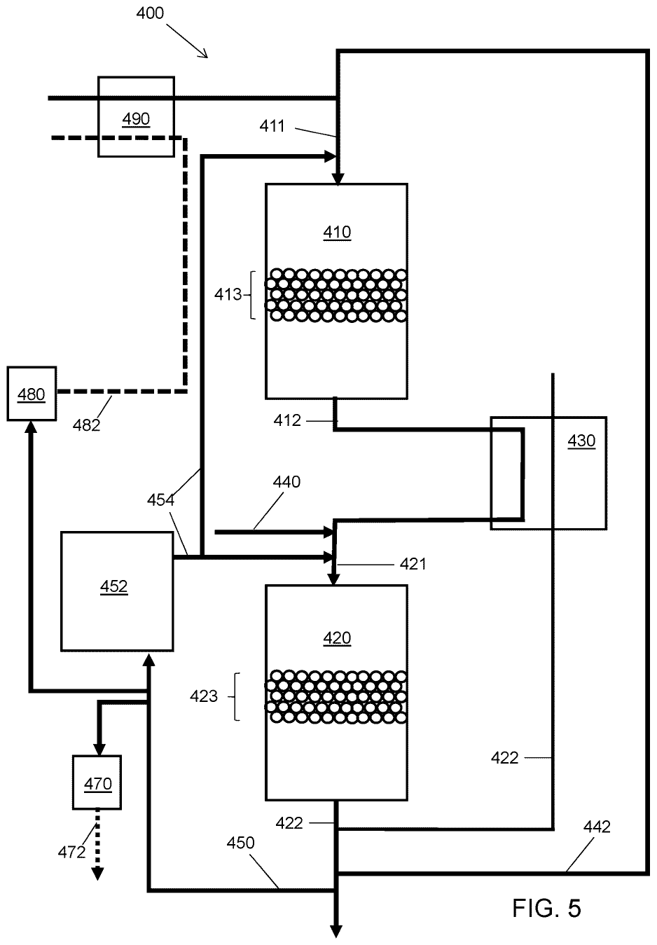

Light hydrocarbons, while often not a desired portion of a Fischer-Tropsch product to be used as a fuel or a lubricant, can themselves be useful for a number of purposes. Accordingly, in various embodiments, the process further includes separating at least a portion of C₁-C₄ hydrocarbons from the second product stream to provide a light hydrocarbon stream. The light hydrocarbon stream can, for example, be recycled to the first feed stream or the second feed stream. For example, in the process 200 of FIG. 3, light hydrocarbons can be provided as part of the recycle stream 236, which becomes part of the second feed stream 221. In the process 300 of FIG. 4, light hydrocarbons can be provided as part of the recycle stream 336, which becomes part of the first feed stream 311. In the process 400 of FIG. 5, light hydrocarbons are recycled via recycle stream 442 to first feed stream 411.

There are other uses for the light hydrocarbon stream. For example, in some embodiments, the process further comprises oxidizing at least a portion of the light hydrocarbon stream to provide a CO- and/or CO₂-containing partial oxidation (pOX) stream, and including at least a portion of the pOX stream in the first feed stream and/or the second feed stream. An example of such a process is shown schematically in FIG. 5, in which the process 400, the first feed stream 411, the first product stream 412, the reverse water-gas shift catalyst 413, the second feed stream 421, the second product stream 422 and the Fischer-Tropsch catalyst 423 can be as otherwise described herein. Here, the process includes oxidizing at least a portion of the light hydrocarbon stream 450) in a partial oxidation reaction zone 452 to provide a CO- and/or CO₂ containing pOX stream, and including at least a portion of the pOX stream 454 stream in the first feed stream 411 and/or the second feed stream 421.

Other methods can be used to provide CO and/or CO₂ from the light hydrocarbon stream. For example, reforming techniques such as steam reforming and autothermal reforming can be used to provide CO by reaction of hydrocarbons with water. Accordingly, in various embodiments, the process further comprises reforming (e.g., steam reforming and/or autothermal reforming) at least a portion of the light hydrocarbon stream to provide a CO- and/or CO₂-containing reformed stream, and including at least a portion of the reformed stream in the first feed stream and/or the second feed stream. Water separated from the first and/or second product streams can be provided as part of a feed to the reforming described here.

Moreover, the light hydrocarbon stream can be burned to provide heat energy, which can be used to heat various process streams, or to generate electricity. Accordingly, in various embodiments, the process includes burning at least a portion of the light hydrocarbon stream to provide energy, e.g., heat energy or electrical energy. For example, in the process 400 of FIG. 5, a portion of light hydrocarbon stream 450 is burned in a power generation zone (here, in an electrical generator 470), to generate electricity stream 472. In various embodiments, the heat energy may be used to provide the needed heat duty for the reverse water-gas shift process. For example, in the process 400 of FIG. 5, a portion of the light hydrocarbon stream 450 is burned in a power generation zone (here, in a heat generator 480), to generate heat stream 482. The heat stream 482 is conducted to a heat exchange zone 490 to heat the first feed stream 411. Heat energy can similarly be provided to the Fischer-Tropsch reaction. And as the person of ordinary skill in the art will appreciate, other treatments of the light hydrocarbon stream (e.g., partial oxidation) can also provide energy, which can be used, e.g., as described herein.

As with the first product stream, heat can be exchanged from the second product stream to provide heat to, for example, a feed stream or a steam generation zone. For example, in various embodiments, the process further comprises exchanging heat between at least a portion of the second product stream and at least a portion of the first feed stream, thereby cooling at least a portion of the second product stream and heating at least a portion of the first feed stream. In process 300 of FIG. 4, heat is exchanged between at least a portion of the second product stream 322 and first feed stream 311 in a second heat exchange zone 330, thereby cooling the second product stream 322 and heating the first feed stream 311. Of course, heat can also be exchanged from the second product stream to the second feed stream. For example, in various embodiments, the process further comprises exchanging heat between at least a portion of the second product stream and at least a portion of the second feed stream, thereby cooling at least a portion of the second product stream and heating at least a portion of the second feed stream. In process 400 of FIG. 5, heat is exchanged between at least a portion of the second product stream 422 and second feed stream 421 in a second heat exchange zone 430, thereby cooling the second product stream 422 and heating the second feed stream 421. The person of ordinary skill in the art will appreciate that a wide variety of heat exchangers can be used for this purpose.

Of course, any excess heat in the second product stream can be additionally or alternatively used for other purposes. For example, in various embodiments the process further comprises exchanging heat between at least a portion of the second product stream and a steam generation zone, thereby cooling at least a portion of the second product stream and providing heat to the steam generation zone. This is shown in FIG. 4. Here, after heat exchange with the first feed stream 311, the second product stream 322 is conducted to steam generation zone 332, to cool the second product stream 322 and provide heat to the steam generation zone 332. Steam can be generated from the heat provided, and electricity can be generated from the steam (not shown here).

It can be desirable to recycle hydrogen from the second product stream, for example, to the first feed stream and/or the second feed stream. For example, in various embodiments, the process includes recycling at least a portion of H₂ of the second product stream to the second feed stream. For example, in the process of FIG. 3, at least a portion of H₂ of the second product stream (e.g., at least 25%, at least 50%, or at least 75%) can be recycled to the second feed stream 221 via recycle stream 236. In various embodiments, the process includes recycling at least a portion of H₂ of the second product stream to the first feed stream. For example, in the process of FIG. 4, at least a portion of H₂ of the second product stream (e.g., at least 25%, at least 50%, or at least 75%) can be recycled to the first feed stream 311 via recycle stream 336. In various embodiments, at least 25%, e.g., at least 50% of H₂ of the second product stream is recycled to the first feed stream or the second feed stream. In various embodiments, at least 75%, e.g., at least 90% of H₂ of the second product stream is recycled to the first feed stream or the second feed stream.

In some cases, e.g., when H₂ is provided to the second feed stream from an H₂ source other than the first product stream, H₂ from the second product stream can make up most of the H₂ of the first feed stream, e.g., at least 90%, at least 95%, or at least 98% of the H₂ of the first feed stream. This is shown, e.g., in FIG. 5. Here, the primary H₂ input to the process is through stream 440, which becomes part of the second feed stream 421. H₂ of the second product stream is included in recycle stream 442, which becomes part of first feed stream 411.

Similarly, it can be desirable to recycle CO of the second product stream, for example, to the first feed stream and/or the second feed stream. For example, in various embodiments, the process includes recycling at least a portion of CO of the second product stream to the second feed stream. For example, in the process of FIG. 3, at least a portion of CO of the second product stream (e.g., at least 25%, at least 50%, or at least 75%) can be recycled to the second feed stream 221 via recycle stream 236. In various embodiments, the process includes recycling at least a portion of CO of the second product stream to the first feed stream. For example, in the process of FIG. 4, at least a portion of CO of the second product stream (e.g., at least 25%, at least 50%, or at least 75%) can be recycled to the first feed stream 311 via recycle stream 336. In various embodiments, at least 25%, e.g., at least 50% of CO of the second product stream is recycled to the first feed stream or the second feed stream. In various embodiments, at least 75%, e.g., at least 90% of CO of the second product stream is recycled to the first feed stream or the second feed stream.

In many cases, both CO and H₂ of the second product stream will be recycled.

Moreover, when one or more inerts are used in the Fischer-Tropsch process step, it can be desirable to recycle these. For example, in various embodiments, the process includes recycling at least a portion of inerts of the second product stream to the second feed stream. For example, in the process of FIG. 3, at least a portion of inerts of the second product stream (e.g., at least 25%, at least 50%, or at least 75%) can be recycled to the second feed stream 221 via recycle stream 236. In various embodiments, the process includes recycling at least a portion of inerts of the second product stream to the first feed stream. For example, in the process of FIG. 4, at least a portion of inerts of the second product stream (e.g., at least 25%, at least 50%, or at least 75%) can be recycled to the first feed stream 311 via recycle stream 336. In various embodiments, at least 25%, e.g., at least 50% of inerts of the second product stream is recycled to the first feed stream or the second feed stream. In various embodiments, at least 75%, e.g., at least 90% of inerts of the second product stream is recycled to the first feed stream or the second feed stream. In various embodiments, a purge stream can be incorporated with the recycle stream to prevent uncontrolled accumulation of inerts in the recycle stream (not shown here).

Specifically, as CO₂ is the carbon source for the reverse water-gas shift process step, it can be especially desirable to recycle CO₂ to the first feed stream. Accordingly, in various embodiments, the process includes recycling at least a portion (e.g., at least 50%, at least 75%, or at least 90%) of CO₂ of the second product stream to the first feed stream. For example, in the process of FIG. 4, at least a portion of CO₂ of the second product stream (e.g., at least 50%, at least 75%, or at least 90%) can be recycled to the first feed stream 311 via recycle stream 336.

In some cases, e.g., when CO₂ is provided to the second feed stream from a CO₂ source other than the first product stream, CO₂ from the second product stream can make up most of the CO₂ of the first feed stream, e.g., at least 90%, at least 95%, or at least 98% of the CO₂ of the first feed stream. This is shown, e.g., in FIG. 5. Here, the primary CO₂ input to the process is through stream 440, which becomes part of the second feed stream 421. CO₂ of the second product stream is included in recycle stream 442, which becomes part of first feed stream 411.

As noted above, the Fischer-Tropsch process step provides a second product stream that includes C₅₊ hydrocarbons (e.g., unsubstituted hydrocarbons like alkanes and alkenes, and/or oxygenated hydrocarbons such as alcohols). Accordingly, in various embodiments, one or more products are provided from at least a portion of C₅₊ hydrocarbons of the second product stream. The C₅₊ hydrocarbons can be used as the basis of a variety of fuels, e.g., gasoline, diesel, aviation fuel. Other products, like waxes and lubricants, can also be made. And alkenes and oxygenates can be used as feedstocks in a variety of other processes.

The person of ordinary skill in the art will use conventional post-processing techniques to convert the C₅₊ hydrocarbon-containing product to desirable products such as desirable fuels. For example, in various embodiments, the process further includes hydroprocessing at least a portion of C₅₊ hydrocarbons of the second product stream. As the person of ordinary skill in the art will appreciate, hydroprocessing is a treatment of the hydrocarbon stream with hydrogen in the presence of a suitable catalyst. A wide variety of hydroprocessing techniques are known and the person of ordinary skill in the art will apply them here. For example, in the process 300 of FIG. 4, second product stream 322 is hydroprocessed in hydroprocessing reactor 350, to provide a hydroprocessed product stream 352.

As described above, CO₂ and H₂ are substantial inputs to the claimed processes. Advantageously, the present inventors have recognized that each of these can come from renewable or otherwise environmentally responsible sources.

CO₂ can be captured from the environment generally or more directly from processes that form CO₂ (especially in difficult-to-abate sectors). This can make the eventual hydrocarbon product substantially carbon-neutral or of lower carbon intensity. Accordingly, in some embodiments of the disclosure as described herein, at least a part of the CO₂ of the first feed stream and/or the second feed stream is from a renewable source. In some embodiments, at least part (e.g., at least 25%, at least 50%, or at least 75%) of the CO₂ of the first feed stream and/or the second feed stream is from direct air capture. In some embodiments, at least part (e.g., at least 25%, at least 50%, or at least 75%) of the CO₂ of the first feed stream and/or the second feed stream is from a manufacturing plant such as a bioethanol plant (e.g., CO₂ produced fermentation), a steel plant, or a cement plant. Accordingly, the rWGS-Fischer Tropsch integrated processes of the disclosure as described herein can be not only carbon neutral, but in some cases a net consumer of carbon dioxide. These benefits in particular make the integrated processes highly attractive for decarbonizing transportation fuels, for both automotive and aviation sectors, since the carbon monoxide produced in the rWGS reaction can be readily utilized by well-established technologies to synthesize liquid hydrocarbon fuels by Fischer-Tropsch processes.

Similarly, H₂ can be provided from environmentally-responsible sources. In some embodiments, at least a part of the H₂ of the first feed stream and/or the second feed stream is from a renewable source. For example, in various embodiments, at least part (e.g., at least 25%, at least 50%, or at least 75%) of the H₂ of the first feed stream and/or the second feed stream can be so-called “green” hydrogen, e.g., produced from the electrolysis of water operated using renewable electricity (such as wind, solar, or hydro-electric power). In some embodiments, at least part (e.g., at least 25%, at least 50%, or at least 75%) of the H₂ of the first feed stream and/or the second feed stream may be from a so-called “blue” source, e.g., from a natural gas reforming process with carbon capture. Of course, other sources of H₂ can be used in part or in full. For example, in some embodiments, at least part (e.g., at least 25%, at least 50%, or at least 75%) of the H₂ of the first feed stream and/or the second feed stream is grey hydrogen, black hydrogen, brown hydrogen, pink hydrogen, turquoise hydrogen, yellow hydrogen, and/or white hydrogen.

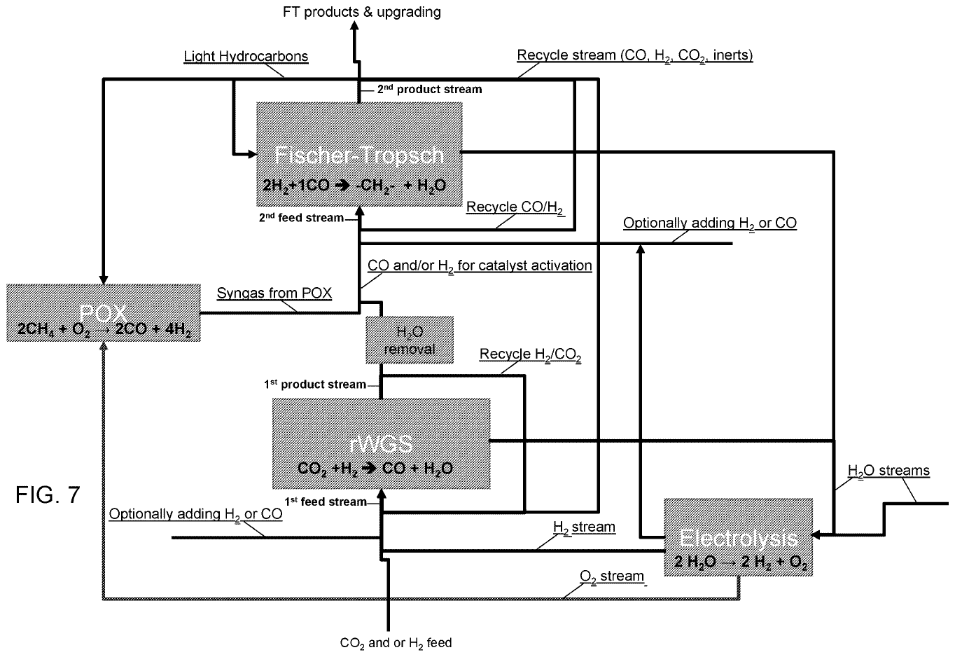

The present inventors have noted that electrolysis of water is a desirable way to provide hydrogen to the claimed processes. Accordingly, in some embodiments, the process includes providing at least a portion of H₂ to the first feed stream and/or the second feed stream by electrolysis of water. In some embodiments, the electrolysis of water is performed using at least partially electricity from a renewable source, e.g., to provide so-called “green hydrogen.” However, the present inventors have noted that electricity can be generated as part of the claimed process, e.g., using heat exchange from the first or second product stream, or by burning light hydrocarbons as described above. In some embodiments, the electrolysis of water is performed using at least partially electricity generated according to the processes as described herein. For example, in the process 200 of FIG. 3, water 262 separated from the first product stream is electrolyzed in electrolyzer 260, using electricity 264 generated from steam made in the steam generation zone 232 by heat exchange from the first product stream. H₂ generated in the electrolysis is provided via stream 265 to the first feed stream. In some embodiments, at least a portion of O₂ generated in the electrolysis is provided to a partial oxidation reaction zone as described herein and as shown in the embodiment of FIG. 7. Hydrogen from electrolysis can also burned to provide heat energy, e.g., which can be used to heat the first feed stream.

The processes described herein can be operated in a wide variety of reactor systems. In some embodiments, the first reaction zone (i.e., in which the reverse water-gas shift process step is performed) comprises a first reactor in which a reverse water-gas shift catalyst is disposed, and the second reaction zone (i.e., in which the Fischer-Tropsch process step is performed) comprises a second reactor in which the Fischer-Tropsch catalyst is disposed. Examples of such processes are shown schematically in FIGS. 1, 2, and 4. In these examples, the process (100, 200, 400) is performed in a reactor system that includes a first reactor (110, 210, 410) in which a reverse water-gas shift catalyst (113, 213, 413) is disposed, and a second reactor (120, 220, 420) in which the Fischer-Tropsch catalyst (123, 223, 423) is disposed. The reactors used for the integrated process of the present disclosure as described herein are not particularly limited, and the person of ordinary skill in the art will be able to select an appropriate reactor.

But other embodiments are possible. For example, in some embodiments, the process is performed in a reactor system comprising first catalyst bed in which the reverse water-gas shift catalyst is disposed, and wherein the second reaction zone comprises a second catalyst bed in which the Fischer-Tropsch catalyst is disposed. In some embodiments, the first reactor bed and the second reactor bed are disposed within the same reactor. Such a configuration is shown in FIG. 4, in which the reverse water-gas shift catalyst 313 is disposed in a first catalyst bed 314, and the Fischer-Tropsch catalyst 323 is disposed in a second catalyst bed 324. Here, the catalyst beds 314 and 324 are in the same reactor 305, with process gases flowing between them. Such a configuration can be especially desirable when the first temperature and the second temperature are relatively close to one another.

In various embodiments, the process is performed in a reactor system comprising one or more first catalyst containers in which the reverse water-gas shift catalyst is disposed, and wherein the second reaction zone comprises one or more second catalyst containers in which the Fischer-Tropsch catalyst is disposed. These can be provided in the same reactor, such as described above with respect to catalyst beds.

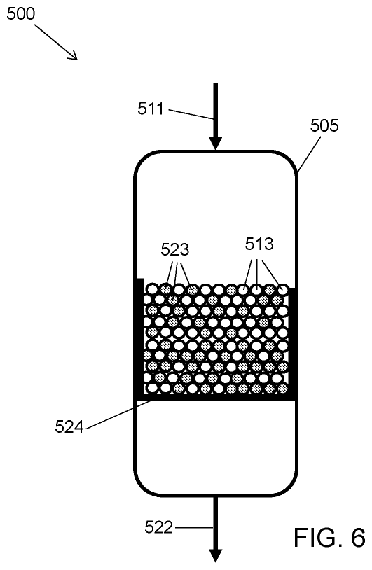

As noted above, the reverse water-gas shift process step using the platinum, palladium, and/or gold catalysts described herein and the Fischer-Tropsch process step can be performed under similar conditions. Accordingly, in various embodiments, the reverse water-gas shift catalyst and the Fischer-Tropsch catalyst can be provided together in the same catalyst bed, e.g., mixed together. Such an embodiment is shown in FIG. 6. Here, the process 500 is performed in a reactor system that includes a reactor 505 in which the reverse water-gas shift catalyst 513 and the Fischer-Tropsch catalyst 523 are mixed together in a single catalyst bed 524. Here, first feed stream 511 and second product stream 522 can be substantially as described herein. The first product stream and the second feed stream are understood to be the mixture of process gases within the mixed catalysts.

In the embodiments particularly-described above, separate rWGS and Fischer-Tropsch catalysts are used, e.g., in separate reactors, in separate regions of the same reactor, or even comingled in the same region of a reactor.

However, the present inventors also note that there are certain commonalities between the rWGS catalysts described herein and certain Fischer-Tropsch catalysts. For example, as the person of ordinary skill in the art would appreciate, manganese is a common modifier used in Fischer-Tropsch catalysts, especially those based on cobalt. The present inventors also note that similar supports can be used for each.

Accordingly, in addition to the configurations described above, the present inventors contemplate the provision of a single bifunctional catalyst with both reverse water-gas shift activity and Fischer-Tropsch activity. Such a bifunctional catalyst includes both rWGS-active catalyst metal and the Fischer-Tropsch active catalyst metal in the same body. The person or ordinary skill in the art will appreciate that both the rWGS catalysts and the Fischer-Tropsch catalysts are supported catalyst, e.g., metal oxide supported catalyst. As such, in various embodiments of the present disclosure, the rWGS-active catalyst metals and Fischer-Tropsch active catalyst metals can be provided together on the same support to provide a bifunctional catalyst. For example, in some embodiments, the supports of bifunctional catalyst are provided themselves as discrete body of material, e.g., as porous particles, pellets, or shaped extrudates, with the rWGS-active catalyst metals and the FT-active catalyst metals provided thereon to provide a bifunctional catalyst. The rWGS-active catalyst metals and the FT-active catalyst metals may be homogenous distributed throughout the support or may be distributed in discrete areas throughout the support. However, in other embodiments, the bifunctional catalyst of the disclosure can itself be formed as a layer on an underlying substrate. For example, in some embodiments, the bifunctional catalyst is formed from a layer of rWGS-active catalyst metals and a layer of FT-active catalyst metals on an underlying substrate. The rWGS-active catalyst metals and the FT-active catalyst metals may be homogeneously distributed on the underlying substrate. In other embodiments, the rWGS-active catalyst metals and the FT-active catalyst metals may be in discrete areas on the underlying substrate. The underlying substrate is not particularly limited. It can be formed of, e.g., a metal or metal oxide, and can itself be provided in a number of forms, such as particles, pellets, shaped extrudates, or monoliths.

The bifunctional catalyst includes a support material, the rWGS-active catalyst metals as described herein, and the Fischer-Tropsch active catalyst metals as described herein. For example, the bifunctional catalyst includes a support that is a metal oxide support as described herein, at least one of platinum, palladium, and gold, manganese, and at least one of cobalt, iron, rhodium, and ruthenium. In some embodiments of present disclosure, the bifunctional catalyst includes a support that is a metal oxide support as described herein, at least one of platinum, palladium, and gold, manganese, and cobalt. In some embodiments of the present disclosure, the bifunctional catalyst includes a support comprising at least one of titanium oxide, zirconium oxide, cerium oxide, or aluminum oxide, at least one of platinum, palladium, and gold, manganese, and cobalt. In some embodiments, the bifunctional catalyst includes a titanium oxide support, at least one of platinum, palladium, and gold, manganese, and cobalt. In some embodiments, the bifunctional catalyst includes a titanium oxide support, platinum, manganese, and cobalt. In some embodiments, the bifunctional catalyst includes a titanium oxide support, palladium, manganese, and cobalt. In some embodiments, the bifunctional catalyst includes a titanium oxide support, gold, manganese, and cobalt. For example, in some embodiments, the bifunctional catalyst includes a titanium oxide support, at least one of platinum, palladium, and gold, present in an amount in the range of 0.05 to 10 wt %, manganese, present in an amount in the range of 0.5 to 20 wt %, and cobalt, present in an amount in the range of 7-25 wt %. In some embodiments, the bifunctional catalyst includes a titanium oxide support, platinum, present in an amount in the range of 0.05 to 10 wt %, manganese present in an amount in the range of 0.5 to 20 wt %, and cobalt, present in an amount in the range of 7-25 wt %. In some embodiments, the bifunctional catalyst includes a titanium oxide support, palladium, present in an amount in the range of 0.05 to 10 wt %, manganese present in an amount in the range of 0.5 to 20 wt %, and cobalt, present in an amount in the range of 7-25 wt %. In some embodiments, the bifunctional catalyst includes a titanium oxide support, gold, present in an amount in the range of 0.05 to 10 wt %, manganese present in an amount in the range of 0.5 to 20 wt %, and cobalt, present in an amount in the range of 7-25 wt %.

The ratio of rWGS-active catalyst metals to FT-active catalyst metals in the bifunctional catalyst is not particularly limited and the person of ordinary skill in the art would be able to select an appropriate ratio. For example, in some embodiments, the ratio of rWGS-active catalyst metals to FT-active catalyst metals in the bifunctional catalyst is at least 0.1:1. In various embodiments, the ratio of rWGS-active catalyst metals to FT-active catalyst metals in the bifunctional catalyst is at least 0.2:1, or 0.5, or 1:1.

Such catalysts can be used in embodiments like those described with respect to FIG. 6. The person of ordinary skill in the art will select reaction conditions that provide the appropriate balance of reverse water-gas shift activity and Fischer-Tropsch activity.

FIG. 7 is a schematic depiction of another integrated process according to the disclosure. Here, the reverse water-gas shift and Fischer-Tropsch process steps are integrated together with partial oxidation of light hydrocarbons to provide CO and H₂ to the Fischer-Tropsch process step; electrolysis to provide H₂ for the reverse water-gas shift process step and O₂ for the partial oxidation; and various recycles and optional feeds, as described throughout the present specification.

The person of ordinary skill in the art will provide the materials and perform the processes described herein based on the general disclosure above, and with reference to the Examples below.

### EXAMPLES

The Examples that follow are illustrative of specific embodiments of the catalysts and processes of the disclosure, and various uses thereof. They are set forth for explanatory purposes only and are not to be taken as limiting the scope of the disclosure.

### Example 1. Modelling

The present inventors performed modelling of various equilibrium conditions of the reverse water-gas shift reaction. The predicted carbon dioxide conversion and product composition of the rWGS reaction (Equation 1) in competition with the Sabatier reaction (Equation 3) and the CO methanation reaction (Equation 4) was calculated over the temperature range of 400-800° C., based on thermodynamic equilibrium. From the modeling, the carbon monoxide selectivity increased at temperatures greater than 600° C., while methane selectivity decreased at higher temperatures.

From these results, it is clear that the Sabatier reaction (Equation (3) and CO methanation (Equation (4)) side reactions are exothermic and favored at lower temperatures, while the rWGS reaction (Equation (1)) is endothermic and favored at higher temperatures. However, other carbon-producing side reactions, not accounted for in this example, can occur at high temperatures. As such, the present inventors have investigated catalysts that operate at a middling temperature. These catalysts are discussed in more detail below.

### Example 2 Catalyst Preparation

A conventional impregnation process was used to prepare catalysts before testing their viability for reverse water-gas shift reactions. The supports used are described in Table 1.

TABLE 1

| Chemical | Supplier | Pore Volume | |
|---|---|---|---|
| Cerium(IV) oxide | Sigma-Aldrich | 0.1 | mL/g |
| γ-Alumina | Saint Gobain | 0.83 | mL/g |
| Titania P-25 Aeroxide | Evonik | 0.5 | mL/g |

To prepare the catalyst, a solution of the active metal (e.g., Pd, Pt, or Au) and manganese acetate tetrahydrate (purity 99.9%, Fisher Chemicals) was prepared in deionized water. For the solution containing platinum, a platinum (II) nitrate solution, at 17.5 wt % Pt and 99.95% purity, from Umicore was used. For the solution containing palladium, a palladium (II) nitration solution, with 23.9 wt % Pd and 99.95% metal purity, from Umicore was used. For the solution containing gold, gold (III) hydroxide (79.1% Au measured after reduction with hydrazine dihydrate), from Sigma-Aldrich was used. The solution of active metal salt and manganese was added to the support powder. The amount of support added is based on the amount of the water on a mass basis so that the ratio of water:support is 3:1. The slurry was then stirred at room temperature for 4 hours. Excess water was then evaporated using a stirring drybath at a temperature of 60° C. The resulting catalyst precursor powder was then dried for 24 hours at 90° C. in a drying oven.

That catalyst precursor powder was then subjected to calcination by evenly spreading out the powder in a crucible. The crucible is placed in a calcination furnace and the temperature is increased from ambient to 120° C. at a rate of 10° C. per minute. The temperature was then held at 120° C. for 1 hour, and then increased from 120° C. to 500° C. at a rate of 2° C. per minute. The temperature was held at 500° C. for 4 hours, and then cooled to ambient temperature. The resulting catalysts were then tested for its viability for reverse water-gas shift reactions.

### Example 3. Performance of Platinum and Palladium Catalysts

Catalysts prepared by methods as described herein were then tested for their catalytic performance for reverse water-gas shift reactions. The catalysts were ceria supported catalysts that contained either platinum or palladium at 0.25 wt %, 0.5 wt %, and 2 wt % and manganese at either 0 wt %, 1 wt %, or 5 wt %. To test the catalytic performance of these catalysts, 20 μL of the catalyst diluted with SiC F100 to provide a ratio of 1:10 was loaded into a 3 mm ID ceramic tube reactor, resulting in a 0.22 mL catalyst bed with a zone height of 31.1 mm. Prior to performing the rWGS reaction, the catalysts were activated at 590° C. for 5 hours in a 97% hydrogen and 3% argon atmosphere. Then, the catalysts were contacted at a temperature of 600° C. with a feed stream including H₂ and CO₂ at a ratio of 2:1. The total pressure was kept at 30 barg. For trials 1-2, 4, and 6-8 the GHSV was kept at 29,500 h⁻¹, while for trials 3 and 5, the GHSV was 1,200,000 h⁻¹. The catalytic performance was analyzed by detecting the gas composition of the reactor outlet feed using a multi-detector gas chromatograph. The catalytic performance of these catalysts are shown in Table 2. In Table 2, and Tables 3-7 below, the amount of platinum, palladium, and/or manganese present in the catalyst are shown in parenthesis. These numbers are in weight percent and based on the total weight of the catalyst. For example, CeO₂Pt(0.5)Mn(1) corresponds to a catalyst with 0.5 wt % Pt, 1 wt % Mn, and 98.5 wt % CeO₂.

TABLE 2

| | | | CO | CO₂ |
|---|---|---|---|---|
| Trial | | CH₄ STY | Selectivity | conversion |
| No. | Catalyst | (kg l⁻¹h⁻¹) | (%) | (%) |
| 1 | CeO₂Pt(0.5) | 1.40 | 55.3 | 47.4 |
| 2 | CeO₂Pt(0.5)Mn(1) | 0.09 | 97.1 | 50.5 |
| 3 | CeO₂Pt(0.25)Mn(5) | — | 99.5 | 35.8 |
| 4 | CeO₂Pt(0.5)Mn(5) | 0.07 | 97.8 | 50.7 |
| 5 | CeO₂Pt(2)Mn(5) | — | 99.7 | 48.1 |
| 6 | CeO₂Pd(0.5) | 0.79 | 74.2 | 48.8 |
| 7 | CeO₂Pd(0.5)Mn(1) | 0.07 | 97.7 | 50.8 |
| 8 | CeO₂Pd(0.5)Mn(5) | 0.03 | 99.0 | 50.3 |

The catalyst including 0.5 wt % platinum or palladium demonstrate very high CO selectivity, with the addition of manganese to the catalyst result in an increase in both catalyst activity and CO selectivity.

To further investigate these catalysts, the CeO₂Pd(0.5)Mn(5), CeO₂Pd(0.5)Mn(1), CeO₂Pt(0.5)Mn(1) and CeO₂Pt(0.5)Mn(5) catalysts were also measured under a different temperatures and pressures. Trials 9-20 were conducted at a H₂:CO₂ molar ratio of 2:1 and GHSV of 16,000 h⁻¹, and trial 21 was conducted at a H₂:CO₂ molar ratio of 2:1 and GHSV of 1,200,000 h⁻¹. These results are shown in Table 3.

TABLE 3

| | | Temper- | | CO | CO₂ |
|---|---|---|---|---|---|
| Trial | | ature | Pressure | Selectivity | conversion |
| No. | Catalyst | (° C.) | (barg) | (%) | (%) |
| 9 | CeO₂Pt(0.5)Mn(1) | 300 | 10 | 98.7 | 4.4 |
| 10 | CeO₂Pt(0.5)Mn(5) | 300 | 10 | 99.3 | 4.1 |
| 11 | CeO₂Pd(0.5)Mn(1) | 300 | 10 | 97.3 | 3.9 |
| 12 | CeO₂Pd(0.5)Mn(5) | 300 | 10 | 98.0 | 4.4 |
| 13 | CeO₂Pt(0.5)Mn(5) | 400 | 10 | 99.9 | 36.6 |
| 14 | CeO₂Pd(0.5)Mn(5) | 400 | 10 | 99.4 | 10.2 |
| 15 | CeO₂Pt(0.5)Mn(5) | 500 | 10 | 99.7 | 29.6 |
| 16 | CeO₂Pd(0.5)Mn(5) | 500 | 10 | 99.4 | 24.3 |
| 17 | CeO₂Pt(0.5)Mn(5) | 500 | 10 | 99.4 | 41.0 |
| 18 | CeO₂Pd(0.5)Mn(5) | 500 | 10 | 99.2 | 34.8 |
| 19 | CeO₂Pt(0.5)Mn(5) | 500 | 20 | 99.0 | 41.0 |
| 20 | CeO₂Pd(0.5)Mn(5) | 500 | 20 | 98.6 | 36.2 |
| 21 | CeO₂Pt(0.5)Mn(5) | 700 | 30 | 99.4 | 56.0 |

As shown in Table 3, the catalysts show excellent CO selectivity and that remains consistent across the range of process conditions tested. The high selectivity of CO demonstrated in the trials above provide an effluent stream suitable for integration with other processes.

### Example 4. Impact of Manganese on Ceria Supported Platinum Catalysts

To investigate the effect of manganese on the supported reverse water-gas shift catalysts, ceria supported platinum catalysts with and without manganese were measured. Platinum was present in the catalyst at concentrations of 0.1 wt %, 0.5 wt %, or 1 wt % with and without manganese present at 5 wt %. Ceria itself was also measured, as well as a 5 wt % manganese on ceria catalyst without platinum. These catalysts were prepared by methods as described herein. To test the catalytic performance of these catalysts, a Meryer quartz reactor was used with 0.1 g of catalysts at a density of catalyst of around 2 g/mL. A total flowrate of 37.7 mL/min (CO₂ partial pressure of 9.6 mol %, H₂ partial pressure change for different H₂:CO₂ ratio, balanced with Ar). Then, these catalysts were contacted at two different temperatures with a feed stream of H₂ and CO₂, present at a mole ratio of 3:1, under a pressure of 1 bar, and a GHSV of 34,560 h⁻¹ to conduct the rWGS reaction. The catalytic performance was analyzed by detecting the gas composition of the reactor outlet feed using a multi-detector gas chromatograph. The results are shown in Table 4.

TABLE 4

| | | | CO | CO₂ |
|---|---|---|---|---|
| Trial | | Temperature | Selectivity | conversion |
| No. | Catalyst | (° C.) | (%) | (%) |
| 22 | CeO₂ | 500 | >99.9 | 5.2 |
| 23 | CeO₂Mn(5) | 500 | >99.9 | 17.5 |
| 24 | CeO₂Pt(0.1) | 500 | 99.5 | 36.5 |
| 25 | CeO₂Pt(0.1)Mn(5) | 500 | >99.9 | 31.7 |
| 26 | CeO₂Pt(0.5) | 500 | 94.6 | 46.0 |
| 27 | CeO₂Pt(0.5)Mn(5) | 500 | >99.9 | 43.9 |
| 28 | CeO₂Pt(1) | 500 | 79.8 | 48.3 |
| 29 | CeO₂Pt(1)Mn(5) | 500 | >99.9 | 45.7 |
| 30 | CeO₂ | 600 | >99.9 | 26.7 |
| 31 | CeO₂Mn(5) | 600 | >99.9 | 47.3 |
| 32 | CeO₂Pt(0.1) | 600 | 98.7 | 54.1 |
| 33 | CeO₂Pt(0.1)Mn(5) | 600 | >99.9 | 53.4 |
| 34 | CeO₂Pt(0.5) | 600 | 86.0 | 58.1 |
| 35 | CeO₂Pt(0.5)Mn(5) | 600 | 99.8 | 58.0 |
| 36 | CeO₂Pt(1) | 600 | 70.1 | 59.0 |
| 37 | CeO₂Pt(1)Mn(5) | 600 | 99.7 | 58.6 |

Table 4 shows that one route to increase catalyst activity is by increasing platinum loading. However, this is accompanied with a decrease in CO selectivity. When up to 5 wt % manganese, without platinum is disposed on the supported catalyst, CO can be recovered to greater than 99% at conditions tested, albeit with loss in activity. But with the addition of 0.1 wt % to 1 wt % platinum to the 5 wt % manganese on ceria catalyst, an increase in the CO selectivity and CO₂ conversion are observed as demonstrated in Table 4. Some loss of catalyst activity is accompanied with the large gains in CO selectivity. However, without being bound by theory, the present inventors believe that conversion may drop due to less methanation occurring.

To further investigate the impact of ceria supported manganese catalyst with platinum present at 0.5 wt % or 1 wt % with and without manganese at 5 wt % were contacted at two different temperatures with feed streams with high H₂:CO₂ ratios. These trials were conducted at a pressure of 1 barg and a GHSV of 2250 h⁻¹. Table 5 reports these results.

TABLE 5

| | | Temper- | | CO | CO₂ |
|---|---|---|---|---|---|
| Trial | | ature | | Selectivity | conversion |
| No. | Catalyst | (° C.) | H₂:CO₂ | (%) | (%) |
| 38 | CeO₂Pt(0.5) | 500 | 9:1 | 75.6 | 68.0 |
| 38 | CeO₂Pt(0.5)Mn(5) | 500 | 9:1 | 99.8 | 62.1 |
| 40 | CeO₂Pt(0.5) | 500 | 6:1 | 83.7 | 61.7 |
| 41 | CeO₂Pt(0.5)Mn(5) | 500 | 6:1 | 99.9 | 57.0 |
| 42 | CeO₂Pt(0.5) | 600 | 9:1 | 54.6 | 80.3 |
| 43 | CeO₂Pt(0.5)Mn(5) | 600 | 9:1 | 98.9 | 76.5 |
| 44 | CeO₂Pt(0.5) | 600 | 6:1 | 68.3 | 73.5 |
| 45 | CeO₂Pt(0.5)Mn(5) | 600 | 6:1 | 99.3 | 71.5 |
| 46 | CeO₂Pt(1) | 500 | 9:1 | 41.4 | 74.3 |
| 47 | CeO₂Pt(1)Mn(5) | 500 | 9:1 | 99.8 | 66.5 |
| 48 | CeO₂Pt(1) | 500 | 6:1 | 54.3 | 65.7 |
| 49 | CeO₂Pt(1)Mn(5) | 500 | 6:1 | 99.9 | 60.7 |
| 50 | CeO₂Pt(1) | 600 | 9:1 | 34.2 | 84.1 |
| 51 | CeO₂Pt(1)Mn(5) | 600 | 9:1 | 98.6 | 77.9 |
| 52 | CeO₂Pt(1) | 600 | 6:1 | 52.3 | 75.6 |
| 53 | CeO₂Pt(1)Mn(5) | 600 | 6:1 | 99.0 | 72.6 |

Table 5 shows that the presence of Mn in the catalyst preserves the CO selectivity even at very high H₂:CO₂ ratios. When the catalyst with manganese and platinum or palladium supported on ceria are used, it is observed that the CO selectivity remains robust with values of greater than 99% observed, as shown in Table 5.

### Example 5. Impact of Support on Catalyst Performance

The impact of the support on catalyst performance was also evaluated by evaluating platinum and manganese supported catalysts on a variety of catalytic supports (ceria, alumina, and titania). These catalysts contain 0.5 wt % platinum and 5 wt % manganese. As with the previous examples, these catalysts were prepared by the methods as described herein. The reactor setup and catalyst activation as described in Example 4 were also used here. Then, these catalysts were contacted at two different temperatures with a feed stream of H₂ and CO₂, present at a mole ratio of 3:1, under a pressure of 1 barg and a GHSV of 34,560 h⁻¹. The catalytic performance was analyzed by detecting the gas composition of the reactor outlet feed using a multi-detector gas chromatograph. The results are shown in Table 6.

TABLE 6

| | | | CO | CO₂ |
|---|---|---|---|---|
| Trial | | Temperature | Selectivity | conversion |
| No. | Catalyst | (° C.) | (%) | (%) |
| 54 | Al₂O₃Pt(0.5) | 500 | 90.5 | 34.3 |
| 55 | Al₂O₃Pt(0.5)Mn(5) | 500 | 99.9 | 30.8 |
| 56 | TiO₂Pt(0.5) | 500 | 91.8 | 42.9 |
| 57 | TiO₂Pt(0.5)Mn(5) | 500 | >99.9 | 18.3 |
| 58 | CeO₂Pt(0.5) | 500 | 94.6 | 46.0 |
| 59 | CeO₂Pt(0.5)Mn(5) | 500 | >99.9 | 43.9 |
| 60 | Al₂O₃Pt(0.5) | 600 | 82.4 | 56.1 |
| 61 | Al₂O₃Pt(0.5)Mn(5) | 600 | 99.7 | 56.3 |
| 62 | TiO₂Pt(0.5) | 600 | 86.3 | 57.7 |
| 63 | TiO₂Pt(0.5)Mn(5) | 600 | 99.8 | 44.6 |
| 64 | CeO₂Pt(0.5) | 600 | 86.0 | 58.1 |
| 65 | CeO₂Pt(0.5)Mn(5) | 600 | 99.8 | 58.0 |

Table 6 shows that the manganese effect is still viable on a range of supports for platinum catalysts. The addition of 5 wt % manganese to a variety of metal oxide supports (alumina, praseodymium doped ceria, titania, and ceria) loaded with 0.5 wt % platinum increases the CO selectivity, although the catalyst activity decreases. As described above with respect to the results shown in Table 4, without being bound by theory, the present inventors believe that conversion may drop due to less methanation occurring.

### Example 6. Methane Selectivity of Ceria Supported Platinum Catalysts

Catalysts prepared by methods as described herein were then tested for their methane selectivity for reverse water-gas shift reactions. These catalysts are ceria supported catalysts that contain platinum at 0.5 wt % and manganese in either 0 wt %, 1 wt %, or 5 wt %. The catalysts were contacted with a feed stream comprising 6 vol % H₂ and 2 vol % CO₂. The GHSV was 1200 h⁻¹. The amount of catalyst used was 50 mg. The conversion percentage of carbon dioxide and carbon molar selectivity of methane were tracked over the temperature range of 200° C. to 700° C.

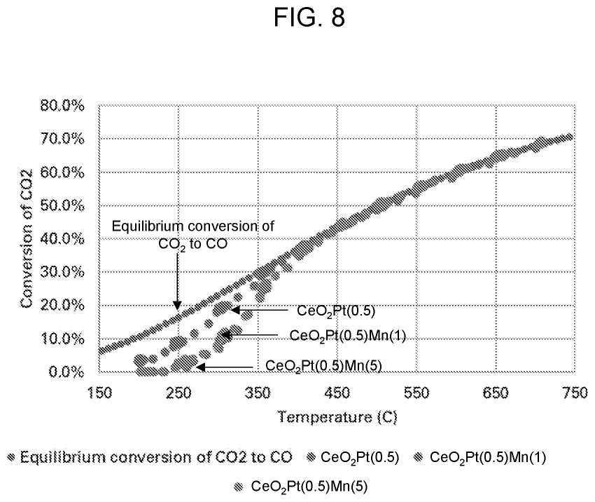

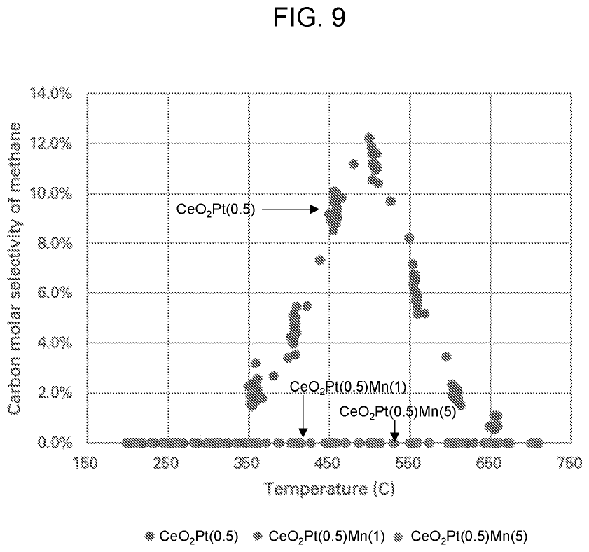

FIG. 8 shows the conversion percentage of CO₂ for the equilibrium conversion of CO₂ to CO along with the reaction conversion for the following catalysts: CeO₂Pt(0.5), CeO₂Pt(0.5)Mn(1), and CeO₂Pt(0.5)Mn(5). FIG. 9 shows the carbon molar selectivity of methane for the three catalysts. From FIG. 8, it can be seen that the presence of the CeO₂Pt(0.5) catalyst lowers the conversion of CO₂ versus the equilibrium conversion at temperatures below 375° C. and that the inclusion of manganese further lowers the conversion. However, without being bound by theory, the present inventors believe that conversion may drop due to less methanation occurring, as shown in FIG. 9. The results suggest that the inclusion of 1 wt % or 5 wt % manganese can modify the product selectivity, such as by decreasing selectivity of methane. For example, as shown in FIG. 9, the methane conversion dropped from 11% to 0% at 500° C. with the addition of manganese to the ceria supported platinum catalyst.

### Example 7. Methane Selectivity of Titania Supported Platinum Catalysts

Catalysts prepared by methods as described herein were then tested for their methane selectivity for reverse water-gas shift reactions. These catalysts are titania supported catalysts that contain platinum at 0.5 wt. % and manganese in either 0 wt. %, 1 wt. %, or 5 wt. %. The catalysts were contacted with a feed stream comprising 6 vol % H₂ and 2 vol % CO₂. The GHSV was 1200 h⁻¹. The amount of catalyst used was 50 mg. The conversion percentage of carbon dioxide and carbon molar selectivity of methane were tracked over the temperature range of 200° C. to 700° C.

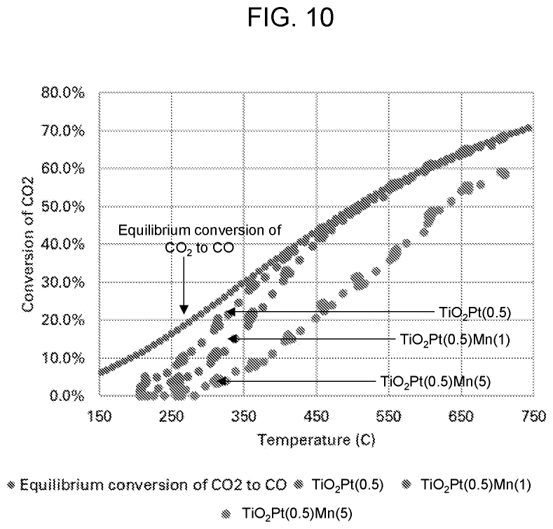

FIG. 10 shows the conversion percentage of CO₂ for the equilibrium conversion of CO₂ to CO, with a TiO₂Pt(0.5) catalyst, with a TiO₂Pt(0.5)Mn(1) catalyst, and with a TiO₂PtMn(5) catalyst. FIG. 10 shows the carbon molar selectivity of methane for the three catalysts. These results show the same effects as shown in FIG. 8 and FIG. 9. The activity shifts to higher temperatures with 1 wt % manganese and further shifts with 5 wt % manganese and the selectivity of methane significantly decreases with 1 wt % manganese and decrease to below the gas chromatograph detection limit with 5 wt % manganese.

### Example 8. Methane Selectivity of Alumina Supported Platinum Catalysts

Catalysts prepared by methods as described herein were then tested for their methane selectivity for reverse water-gas shift reactions. These catalysts are alumina supported catalysts that contain platinum at 0.5 wt. % and manganese in either 0 wt. %, 1 wt. %, or 5 wt. %. The catalysts were contacted with a feed stream comprising 6 vol % H₂ and 2 vol % CO₂. The GHSV was 1200 h⁻¹. The amount of catalyst used was 50 mg catalyst. The conversion percentage of carbon dioxide and carbon molar selectivity of methane were tracked over the temperature range of 200° C. to 700° C., and shown in FIGS. 9 and 10, respectively.

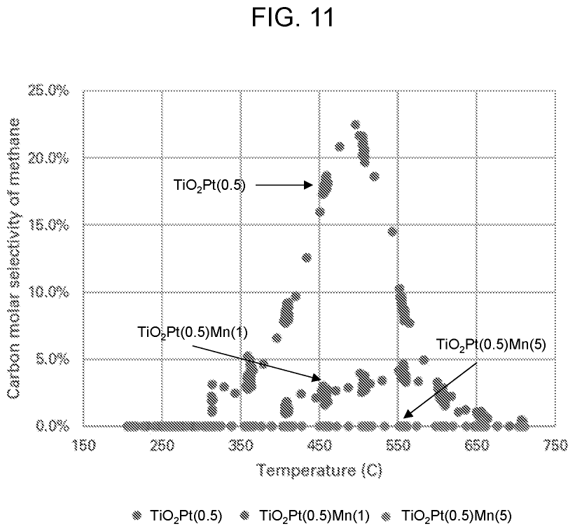

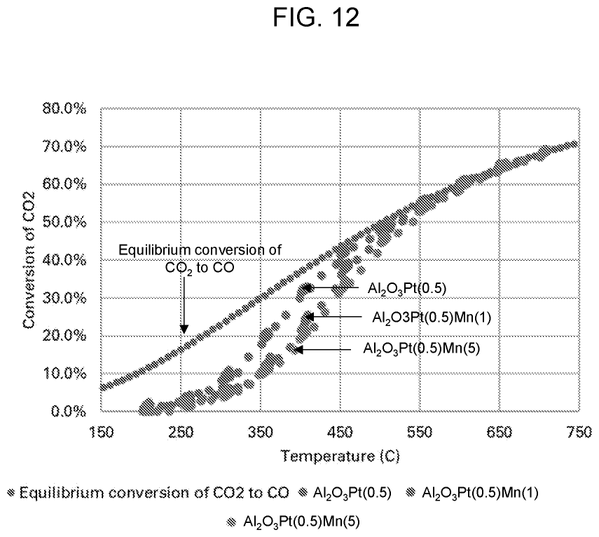

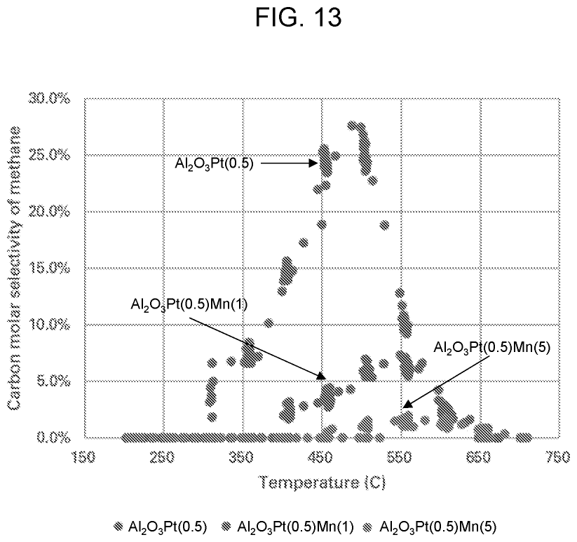

FIG. 12 shows the conversion percentage of CO₂ for the equilibrium conversion of CO₂ to CO, with a Al₂O₃Pt(0.5) catalyst, with a Al₂O₃Pt(0.5)Mn(1) catalyst, and with a Al₂O₃Pt(0.5)Mn(5) catalyst and FIG. 13 shows the carbon molar selectivity of methane for the three catalysts. These results show the same effects as shown in FIGS. 7-11. The activity shifts to higher temperatures with the 1 wt % manganese and further shifts with 5 wt % manganese. Additionally, the selectivity of methane significantly decreases with 1 wt % manganese. With the 5 wt. % manganese, the methane selectivity decreases to 2%.

### Example 9. Impact of Catalyst Activation on Catalyst Performance

Catalysts prepared by methods as described herein were evaluated for the impact of catalyst activation on catalyst performance. Ceria supported catalysts including 0.5 wt % platinum and 5 wt % manganese were activated in a hydrogen atmosphere at three different temperature (400° C., 590° C., and 760° C.) and then used in a rWGS process. To a 3 mm ID ceramic tube reactor, 20 μL of the catalyst diluted with SiC F100 to provide a ratio of 1:10 was loaded, resulting in a 0.22 mL catalyst bed with a zone height of 31.1 mm. Activation of the catalyst was carried out using pure H₂ at a GHSV of 200,000 h⁻¹. The reactor pressure was set to 10 barg, followed by heating the reactor to the desired activation temperature at a rate of 1K/min and holding at that temperature for 5 hours. After activation, the reactors were cooled to the temperature for the rWGS process. Four rWGS reaction temperatures, 400° C., 500° C., 600° C., and 700° C., were evaluated. As the rWGS reaction temperature was reached, the feed stream was introduced followed by setting the reaction pressure. The activated catalysts were contacted with a feed stream of H₂ and CO₂, present at a mole ratio of 2:1, at a pressure of 30 barg and a GHSV of 1,200,000 h⁻¹. The catalytic performance was analyzed by detecting the gas composition of the reactor outlet feed using a multi-detector gas chromatograph. The results are shown in Table 7.

TABLE 7

| | | Activation | Reaction | CO | CO₂ |
|---|---|---|---|---|---|
| | | Temper- | Temper- | Selec- | Con- |
| Trial | | ature | ature | tivity | version |
| No. | Catalyst | (° C.) | (° C.) | (%) | (%) |
| 66 | CeO₂Pt(0.5)Mn(5) | 400 | 400 | 98.4 | 4.3 |
| 67 | CeO₂Pt(0.5)Mn(5) | 400 | 500 | 99.8 | 21.3 |
| 68 | CeO₂Pt(0.5)Mn(5) | 400 | 600 | 99.7 | 39.7 |
| 69 | CeO₂Pt(0.5)Mn(5) | 400 | 700 | 99.5 | 52 |
| 70 | CeO₂Pt(0.5)Mn(5) | 590 | 400 | 98.8 | 4.5 |
| 71 | CeO₂Pt(0.5)Mn(5) | 590 | 500 | 99.8 | 21.8 |
| 72 | CeO₂Pt(0.5)Mn(5) | 590 | 600 | 99.5 | 43.6 |
| 73 | CeO₂Pt(0.5)Mn(5) | 590 | 700 | 99.5 | 51.6 |
| 74 | CeO₂Pt(0.5)Mn(5) | 760 | 400 | 98.6 | 3.2 |
| 75 | CeO₂Pt(0.5)Mn(5) | 760 | 500 | 99.8 | 20.6 |
| 76 | CeO₂Pt(0.5)Mn(5) | 760 | 600 | 99.7 | 42.4 |
| 77 | CeO₂Pt(0.5)Mn(5) | 760 | 700 | 99.3 | 38.3 |

The results of table 7 show consistent CO selectivity and CO₂ conversion independent of activation temperature. Additionally, the results of Table 7 show that while CO selectivity is high (greater than 98%) for all rWGS temperatures measured, the CO₂ conversion increases with increased reaction temperature. However, using a higher activation temperature and a higher rWGS reaction temperature does seem to negatively impact catalyst activity, as seen in trial 70. Overall, the results of Table 7 show that the activation temperature is another variable to adjust the resulting product stream.

### Example 10. Performance of Au Catalysts

Catalysts prepared by methods as described herein were then tested for their catalytic performance for reverse water-gas shift reactions. The catalysts tested were ceria supported catalysts that contain gold at 0.5 wt % and manganese at 0 wt %, 1 wt %, or 5 wt %. To test the catalytic performance of these catalysts, 20 μL of the catalyst diluted with SiC F100 to provide a ratio of 1:10 was loaded into a 3 mm ID ceramic tube reactor, resulting in a 0.22 mL catalyst bed with a zone height of 31.1 mm. Prior to performing the rWGS reaction, the catalysts were activated at 590° C. for 5 hours in a 97% hydrogen and 3% argon atmosphere. Then, the catalysts were contacted at a temperature of 600° C. with a feed stream comprising H₂ and CO₂ at a ratio of 2:1 to conduct the rWGS reaction. The total pressure and GHSV were kept at 30 barg and 29,500 h⁻¹, respectively. The catalytic performance was analyzed by detecting the gas composition of the reactor outlet feed using a multi-detector gas chromatograph. The catalytic performance of these catalysts are shown in Table 8. In Table 8, and Tables 9-11 below, the amount of gold and/or manganese present in the catalysts are shown in parenthesis. These numbers are in weight percent and based on the total weight of the catalyst. For example, CeO₂Au(0.5)Mn(1) corresponds to a catalyst with 0.5 wt % Au, 1 wt % Mn, and 98.5 wt % CeO₂.

TABLE 8

| | | | CO | CO₂ |
|---|---|---|---|---|
| Trial | | CH₄ STY | Selectivity | conversion |
| No. | Catalyst | (kg l⁻¹h⁻¹) | (%) | (%) |
| A1 | CeO₂Au(0.5) | 0.02 | 99.4 | 47.0 |
| A2 | CeO₂Au(0.5)Mn(1) | 0.02 | 99.3 | 49.9 |
| A3 | CeO₂Au(0.5)Mn(5) | 0.01 | 99.6 | 50.2 |

The catalyst including 0.5 wt % gold demonstrate very high selectivity, with the addition of 1 wt % and 5 wt % of manganese to the catalyst resulting in an increase in both catalyst activity and CO selectivity.

Catalysts that contain gold at 0.5 wt % and manganese at 0 wt % and 5 wt % were also tested at 500° C., a H₂:CO₂ ratio of 2:1, a GHSV of 16,000 h⁻¹, and at two different pressures. These results are reported in Table 9.

TABLE 9

| | | | | CO | CO₂ |
|---|---|---|---|---|---|
| Trial | | Pressure | CH₄ STY | Selectivity | conversion |
| No. | Catalyst | (barg) | (kg l⁻¹h⁻¹) | (%) | (%) |
| A4 | CeO₂Au(0.5) | 10 | 0.01 | 97.9 | 15.5 |
| A5 | CeO₂Au(0.5)Mn(5) | 10 | <0.01 | 99.4 | 20.9 |
| A6 | CeO₂Au(0.5) | 20 | 0.02 | 96.5 | 18 |
| A7 | CeO₂Au(0.5)Mn(5) | 20 | 0.01 | 99.1 | 25.1 |

Table 9 shows that the ceria supported manganese and gold catalysts demonstrate very high CO selectivity at other operating conditions.

### Example 11. Impact of Manganese on Ceria Supported Gold Catalysts

To investigate the effect of manganese on the supported reverse water-gas shift catalysts, the ceria supported gold catalysts with and without manganese were measured under different temperatures and pressures. Gold was present in the catalyst at a concentration of 0.5 wt % with and without manganese present at 5 wt %. These catalysts were prepared by methods as described herein. The reactor setup and catalyst activation as described in Example 10 were also used here. For the rWGS reaction, trials A8-A13 were conducted at a H₂:CO₂ molar ratio of 2:1 and GHSV of 29,500 h⁻¹. The catalytic performance was analyzed by detecting the gas composition of the reactor outlet feed using a multi-detector gas chromatograph. These results are shown in Table 10.

TABLE 10

| Trial | | Temperature | Pressure | CH₄ STY | CO | CO₂ |
|---|---|---|---|---|---|---|
| No. | Catalyst | (° C.) | (barg) | (kg l⁻¹h⁻¹) | Selectivity (%) | conversion (%) |
| A8 | CeO₂Au(0.5) | 600 | 12 | 0.03 | 98.8 | 43.1 |
| A9 | CeO₂Au(0.5)Mn(5) | 600 | 12 | 0.01 | 99.7 | 49.7 |
| A10 | CeO₂Au(0.5) | 700 | 12 | 0.02 | 99.4 | 58.2 |
| A11 | CeO₂Au(0.5)Mn(5) | 700 | 12 | 0.01 | 99.7 | 58.1 |
| A12 | CeO₂Au(0.5) | 600 | 20 | 0.02 | 99.4 | 45.1 |
| A13 | CeO₂Au(0.5)Mn(5) | 600 | 20 | 0.01 | 99.7 | 49.8 |

From the results of Tables 9 and 10, the catalysts show excellent CO selectivity and the selectivity remains robust across the range of process conditions tested, especially in the temperature between 500° C. and 700° C. The addition of 5 wt % manganese to the catalyst resulted in an increase CO selectivity with a higher overall CO yield observed at 500° C. and 600° C. In trials A10 and A11 conducted at 700° C., a slight increase in CO selectivity is observed.

The CO selectivity of the supported gold catalysts are extremely high throughout the conditions tested. There is a general trend that the CeO₂Au(0.5)Mn(5) catalyst is more selective than the ceria supported gold catalyst without manganese. This suggests that inclusion of manganese in the formulation is beneficial for CO selectivity. Most importantly, the carbon monoxide selectivity does not decrease as the pressure increases from 12 to 20 barg. This is significant since methanation activity typically increases when operating at higher pressures and typically results in a lower CO selectivity. Operating further from equilibrium could help in this situation as well.

### Example 12. Au Catalyst Performance at Lower Temperatures

The performance of the CeO₂Au(0.5)Mn(5) catalyst was evaluated at lower temperatures and different H₂:CO₂ ratios. The catalyst was prepared by the methods as described herein, and the reactor setup and catalyst activation was used as described in Example 10. For the rWGS reaction, trials A14-A16 were conducted at a pressure of 10 barg, while trial A17 was conducted at 20 barg. All trials were conducted at a GHSV of 16,000 h⁻¹. The catalytic performance was analyzed by detecting the gas composition of the reactor outlet feed using a multi-detector gas chromatograph. The results are shown in Table 11.

TABLE 11

| | | Temper- | | CO | CO₂ |
|---|---|---|---|---|---|
| Trial | | ature | | Selectivity | conversion |
| No. | Catalyst | (° C.) | H₂:CO₂ | (%) | (%) |
| A14 | CeO₂Au(0.5)Mn(5) | 400 | 2:1 | 96.9 | 1.2 |
| A15 | CeO₂Au(0.5)Mn(5) | 500 | 1:1 | 99.6 | 13.0 |
| A16 | CeO₂Au(0.5)Mn(5) | 500 | 2:1 | 99.4 | 20.9 |
| A17 | CeO₂Au(0.5)Mn(5) | 500 | 2:1 | 99.1 | 25.1 |

At the conditions tested in Table 11, the catalyst shows high CO selectivity. Under the conditions used, the catalysts provide an effluent stream suitable for integration with other processes.

Additional aspects of the disclosure are provided by the following enumerated embodiments, which may be combined in any number and in any combination that is not logically or technically inconsistent.
Embodiment 1. A supported reverse water-gas shift catalyst comprising:
   a support that is a cerium oxide support, a titanium oxide support, an aluminum oxide support, a zirconium oxide support, or a mixed oxide support comprising a mixture of two or more of cerium oxide, titanium oxide, aluminum oxide, and zirconium oxide;
   at least one of platinum, palladium, and gold, present in an amount in the range of 0.05 to 10 wt % of the catalyst, based on the total weight of the catalyst; and
   manganese, present in an amount in the range of 0.5 to 20 wt % of the catalyst, based on the total weight of the catalyst.
Embodiment 2. The catalyst of embodiment 1, wherein the support makes up at least 70 wt % (e.g., at least 75 wt %, or 80 wt %, or 85 wt %, or 90 wt %) of the catalyst, on an oxide basis.
Embodiment 3. The catalyst of embodiment 1 or embodiment 2, wherein the support is a cerium oxide support.
Embodiment 4. The catalyst of embodiment 3, wherein at least a surface layer of the cerium oxide support comprises at least 60 wt % cerium oxide, e.g., at least 70 wt % cerium oxide or at least 80 wt % cerium oxide, on an oxide basis.
Embodiment 5. The catalyst of embodiment 3, wherein at least a surface layer of the cerium oxide support comprises at least 90 wt % cerium oxide, e.g., at least 95 wt % cerium oxide, or at least 98 wt % cerium oxide, on an oxide basis.
Embodiment 6. The catalyst of any of embodiments 3-5, wherein the cerium oxide support comprises at least 50 wt % cerium oxide, e.g., at least 60 wt % cerium oxide, or at least 70 wt % cerium oxide, or at least 80 wt % cerium oxide, on an oxide basis.
Embodiment 7. The catalyst of any of embodiments 3-5, wherein the cerium oxide support comprises at least 90 wt % cerium oxide, e.g., at least 95 wt % cerium oxide, or at least 98 wt % cerium oxide, on an oxide basis.
Embodiment 8. The catalyst of embodiment 1 or embodiment 2, wherein the support is a titanium oxide support.
Embodiment 9. The catalyst of embodiment 8, wherein at least a surface layer of the titanium oxide support comprises at least 60 wt % titanium oxide, e.g., at least 70 wt % titanium oxide, or at least 80 wt % titanium oxide, on an oxide basis.
Embodiment 10. The catalyst of embodiment 8, wherein at least a surface layer of the titanium oxide support comprises at least 90 wt % titanium oxide, e.g., at least 95 wt % titanium oxide, or at least 98 wt % titanium oxide, on an oxide basis.
Embodiment 11. The catalyst of any of embodiments 8-10, wherein the titanium oxide support comprises at least 50 wt % titanium oxide, e.g., at least 60 wt % titanium oxide, or at least 70 wt % titanium oxide, or at least 80 wt % titanium oxide, on an oxide basis.
Embodiment 12. The catalyst of any of embodiments 8-10, wherein the titanium oxide support comprises at least 90 wt % titanium oxide, e.g., at least 95 wt % titanium oxide, or at least 98 wt % titanium oxide, on an oxide basis.
Embodiment 13. The catalyst of embodiment 1 or embodiment 2, wherein the support is an aluminum oxide support.
Embodiment 14. The catalyst of embodiment 13, wherein at least a surface layer of the aluminum oxide support comprises at least 60 wt % aluminum oxide, e.g., at least 70 wt % aluminum oxide or at least 80 wt % aluminum oxide, on an oxide basis.
Embodiment 15. The catalyst of embodiment 13, wherein at least a surface layer of the aluminum oxide support comprises at least 90 wt % aluminum oxide, e.g., at least 95 wt % aluminum oxide, or at least 98 wt % aluminum oxide, on an oxide basis.
Embodiment 16. The catalyst of any of embodiments 13-15, wherein the aluminum oxide support comprises at least 50 wt % aluminum oxide, e.g., at least 60 wt % aluminum oxide, or at least 70 wt % aluminum oxide, or at least 80 wt % aluminum oxide, on an oxide basis.
Embodiment 17. The catalyst of any of embodiments 13-15, wherein the aluminum oxide support comprises at least 90 wt % aluminum oxide, e.g., at least 95 wt % aluminum oxide, or at least 98 wt % aluminum oxide, on an oxide basis.
Embodiment 18. The catalyst of embodiment 1 or embodiment 2, wherein the support is a zirconium oxide support.
Embodiment 19. The catalyst of embodiment 18, wherein at least a surface layer of the zirconium oxide support comprises at least 60 wt % zirconium oxide, e.g., at least 70 wt % zirconium oxide or at least 80 wt % zirconium oxide, on an oxide basis.
Embodiment 20. The catalyst of embodiment 18, wherein at least a surface layer of the zirconium oxide support comprises at least 90 wt % zirconium oxide, e.g., at least 95 wt % zirconium oxide, or at least 98 wt % zirconium oxide, on an oxide basis.
Embodiment 21. The catalyst of any of embodiments 18-20, wherein the zirconium oxide support comprises at least 50 wt % zirconium oxide, e.g., at least 60 wt % zirconium oxide, or at least 70 wt % zirconium oxide, or at least 80 wt % zirconium oxide, on an oxide basis.
Embodiment 22. The catalyst of any of embodiments 18-21, wherein the zirconium oxide support comprises at least 90 wt % zirconium oxide, e.g., at least 95 wt % zirconium oxide, or at least 98 wt % zirconium oxide, on an oxide basis.
Embodiment 23. The catalyst of embodiment 1 or embodiment 2, wherein the support is a mixed oxide support having at least a surface layer comprising at least 50 wt % of two or more of cerium oxide, titanium oxide, aluminum oxide, and zirconium oxide, on an oxide basis.
Embodiment 24. The catalyst of any of embodiments 1-23, wherein the support does not include additional metals in a total amount of additional metals in excess of 2 wt %, e.g., in excess of 1 wt % or in excess of 0.5 wt %, on an oxide basis.
Embodiment 25. The catalyst of any of embodiments 1-23, wherein the support includes at least one additional metal.
Embodiment 26. The catalyst of embodiment 25, wherein the total amount of the at least one additional metal is in the range of 0.5-20 wt %, e.g., 1-20 wt %, or 2-20 wt %, or 0.5-15 wt %, or 1-15 wt %, or 2-15 wt %, or 0.5-10 wt %, or 1-10 wt %, or 2-10 wt %, or 0.5-5 wt %, or 1-5 wt %, on an oxide basis.
Embodiment 27. The catalyst of any of embodiments 1-26, wherein the support has a pore volume of at least 0.05 mL/g.
Embodiment 28. The catalyst of any of embodiments 1-27, wherein the support has a pore volume of at most 1.5 mL/g.
Embodiment 29. The catalyst of any of embodiments 1-28, wherein the support has a pore volume in the range of 0.05-1.5 mL/g.
Embodiment 30. The catalyst of any of embodiments 1-29, wherein platinum is present in the catalyst.
Embodiment 31. The catalyst of embodiment 30, wherein platinum is present in the catalyst in an amount in the range of 0.1 to 10 wt %, e.g., in the range of 0.5 to 10 wt %, or 1 to 10 wt %, or 2 to 10 wt %, or 5 to 10 wt %, based on the total weight of the catalyst.
Embodiment 32. The catalyst of embodiment 30, wherein platinum is present in the catalyst in an amount in the range of 0.05 to 7 wt %, e.g., in the range of 0.1 to 7 wt %, or 0.5 to 7 wt %, or 1 to 7 wt %, or 2 to 7 wt %, based on the total weight of the catalyst.
Embodiment 33. The catalyst of embodiment 30, wherein platinum is present in the catalyst in an amount in the range of 0.05 to 5 wt %, e.g., in the range of 0.1 to 5 wt %, or 0.5 to 5 wt %, or 1 to 5 wt %, or 2 to 5 wt %, based on the total weight of the catalyst.
Embodiment 34. The catalyst of embodiment 30, wherein platinum is present in the catalyst in an amount in a range of 0.05 to 2 wt %, e.g., in the range of 0.1 to 2 wt %, or 0.3 to 2 wt %, or 0.5 to 2 wt %, or 1 to 2 wt %, based on the total weight of the catalyst.
Embodiment 35. The catalyst of embodiment 30, wherein platinum is present in the catalyst in an amount in a range of 0.05 to 1.5 wt %, e.g., in the range of 0.1 to 1.5 wt %, or 0.3 to 1.5 wt %, or 0.5 to 1.5 wt %, based on the total weight of the catalyst.
Embodiment 36. The catalyst of embodiment 30, wherein platinum is present in the catalyst in an amount in the range of 0.05 to 1 wt %, e.g., in the range of 0.1 to 1 wt %, or 0.3 to 1 wt %, or 0.5 to 1 wt %, based on the total weight of the catalyst.
Embodiment 37. The catalyst of embodiment 30, wherein platinum is present in the catalyst in an amount in the range of 0.05 to 0.8 wt %, e.g., in the range of 0.1 to 0.8 wt %, or 0.3 to 0.8 wt %, or 0.5 to 0.8 wt %, based on the total weight of the catalyst.
Embodiment 38. The catalyst of any of embodiments 1-37, wherein palladium is present in the catalyst.
Embodiment 39. The catalyst of embodiment 38, wherein palladium is present in the catalyst in an amount in the range of 0.1 to 10 wt %, e.g., in the range of 0.5 to 10 wt %, or 1 to 10 wt %, or 2 to 10 wt %, or 5 to 10 wt %, based on the total weight of the catalyst.
Embodiment 40. The catalyst of embodiment 38, wherein palladium is present in the catalyst in an amount in the range of 0.05 to 7 wt %, e.g., in the range of 0.1 to 7 wt %, or 0.5 to 7 wt %, or 1 to 7 wt %, or 2 to 7 wt %, based on the total weight of the catalyst.
Embodiment 41. The catalyst of embodiment 38, wherein palladium is present in the catalyst in an amount in the range of 0.05 to 5 wt %, e.g., in the range of 0.1 to 5 wt %, or 0.5 to 5 wt %, or 1 to 5 wt %, or 2 to 5 wt %, based on the total weight of the catalyst.
Embodiment 42. The catalyst of embodiment 38, wherein palladium is present in the catalyst in an amount in a range of 0.05 to 2 wt %, e.g., in the range of 0.1 to 2 wt %, or 0.3 to 2 wt %, or 0.5 to 2 wt %, based on the total weight of the catalyst.
Embodiment 43. The catalyst of embodiment 38, wherein palladium is present in the catalyst in an amount in a range of 0.05 to 1.5 wt %, e.g., in the range of 0.1 to 1.5 wt %, or 0.3 to 1.5 wt %, or 0.5 to 1.5 wt %, based on the total weight of the catalyst.
Embodiment 44. The catalyst of embodiment 38, wherein palladium is present in the catalyst in an amount in the range of 0.05 to 1 wt %, e.g., in the range of 0.1 to 1 wt %, or 0.3 to 1 wt %, or 0.5 to 1 wt %, based on the total weight of the catalyst.
Embodiment 45. The catalyst of embodiment 38, wherein palladium is present in the catalyst in an amount in the range of 0.05 to 0.8 wt %, e.g., in the range of 0.1 to 0.8 wt %, or 0.3 to 0.8 wt %, or 0.5 to 0.8 wt %, based on the total weight of the catalyst.
Embodiment 46. The catalyst of any of embodiments 1-45, wherein gold is present in the catalyst.
Embodiment 47. The catalyst of any of embodiment 46, wherein gold is present in the catalyst in an amount in the range of 0.1 to 10 wt %, e.g., in the range of 0.5 to 10 wt %, or 1 to 10 wt %, or 2 to 10 wt %, or 5 to 10 wt %, based on the total weight of the catalyst.
Embodiment 48. The catalyst of any of embodiment 46, wherein gold is present in the catalyst in an amount in the range of 0.05 to 7 wt %, e.g., in the range of 0.1 to 7 wt %, or 0.5 to 7 wt %, or 1 to 7 wt %, or 2 to 7 wt %, based on the total weight of the catalyst.
Embodiment 49. The catalyst of any of embodiment 46, wherein gold is present in the catalyst in an amount in the range of 0.05 to 5 wt %, e.g., in the range of 0.1 to 5 wt %, or 0.5 to 5 wt %, or 1 to 5 wt %, or 2 to 5 wt %, based on the total weight of the catalyst.
Embodiment 50. The catalyst of any of embodiment 46, wherein gold is present in the catalyst in an amount in a range of 0.05 to 2 wt %, e.g., in the range of 0.1 to 2 wt %, or 0.3 to 2 wt %, or 0.5 to 2 wt %, or 1 to 2 wt % based on the total weight of the catalyst.
Embodiment 51. The catalyst of any of embodiment 46, wherein gold is present in the catalyst in an amount in a range of 0.05 to 1.5 wt %, e.g., in the range of 0.1 to 1.5 wt %, or 0.3 to 1.5 wt %, or 0.5 to 1.5 wt %, based on the total weight of the catalyst.
Embodiment 52. The catalyst of any of embodiment 46, wherein gold is present in the catalyst in an amount in the range of 0.05 to 1 wt %, e.g., in the range of 0.1 to 1 wt %, or 0.3 to 1 wt %, or 0.5 to 1 wt %, based on the total weight of the catalyst.
Embodiment 53. The catalyst of any of embodiment 46, wherein gold is present in the catalyst in an amount in the range of 0.05 to 0.8 wt %, e.g., in the range of 0.1 to 0.8 wt %, or 0.3 to 0.8 wt %, or 0.5 to 0.8 wt %, based on the total weight of the catalyst.
Embodiment 54. The catalyst of any of embodiments 1-53, wherein manganese is present in the catalyst in an amount in the range of 0.5 to 15 wt %, e.g., in the range of 0.5 to 12 wt % or 0.5 to 10 wt %, based on the total weight of the catalyst.
Embodiment 55. The catalyst of any of embodiments 1-53, wherein manganese is present in the catalyst in an amount in the range of 1 to 20 wt %, e.g., in the range of 1 to 15 wt %, or 1 to 12 wt %, or 1 to 10 wt %, based on the total weight of the catalyst.
Embodiment 56. The catalyst of any of embodiments 1-53, wherein manganese is present in the catalyst in an amount in the range of 2 to 20 wt %, e.g., in the range of 2 to 15 wt %, or 2 to 12 wt %, or 2 to 10 wt %, based on the total weight of the catalyst.
Embodiment 57. The catalyst of any of embodiments 1-53, wherein manganese is present in the catalyst in an amount in the range of 4 to 20 wt %, e.g., in the range of 4 to 15 wt %, or 4 to 12 wt %, or 4 to 10 wt %, based on the total weight of the catalyst.
Embodiment 58. The catalyst of any of embodiments 1-57, wherein a weight ratio of platinum, palladium, and/or gold to manganese is at least 0.05:1, e.g., at least 0.1:1.
Embodiment 59. The catalyst of any of embodiments 1-58, wherein a weight ratio of platinum, palladium, and/or gold to manganese is at most 5:1, e.g., at most 2:1, or 1:1, or 0.5:1.
Embodiment 60. The catalyst of any of embodiments 1-59, wherein a ratio of platinum, palladium, and/or gold to manganese is in the range of 0.05:1 to 1:1 (e.g., in the range of 0.05:1 to 2:1, or 0.05:1 to 1:1, or 0.05:1 to 0.5:1, or 0.05:1 to 0.3:1, or 0.07:1 to 5:1, or 0.07:1 to 2:1, or 0.07:1 to 1:1, or 0.07:1 to 0.5:1, or 0.07:1 to 0.3:1, or 0.1:1 to 5:1, or 0.1:1 to 2:1, or 0.1:1 to 1:1, or 0.1:1 to 0.5:1, or 0.1:1 to 0.3:1).
Embodiment 61. The catalyst of any of embodiments 1-60, wherein the total amount of cerium, titanium, aluminum, zirconium, manganese, platinum, palladium, and gold in the catalyst is at least 90 wt %, e.g., at least 95 wt % or at least 98 wt % of the catalyst, on a metallic basis.
Embodiment 62. The catalyst of any of embodiments 1-61, wherein the catalyst has an additional metal content (i.e., not Mn, Pd, Pt, Au, Ce, Ti, Al, or Zr) of no more than 10 wt % (e.g., no more than 5 wt %, or no more than 2 wt %, or no more than 1 wt %), based on the total weight of the catalyst.
Embodiment 63. The catalyst of any of embodiments 1-62, wherein the catalyst has a Cu content of no more than 10 wt % (e.g., no more than 5 wt %, or no more than 2 wt %, or no more than 1 wt %), based on the total weight of the catalyst.
Embodiment 64. The catalyst of any of embodiments 1-63, wherein the catalyst has an alkali metal content of no more than 10 wt % (e.g., no more than 5 wt %, or no more than 2 wt %, or no more than 1 wt %), based on the total weight of the catalyst.
Embodiment 65. The catalyst of any of embodiments 1-64, wherein the catalyst has an alkaline-earth metal content of no more than 10 wt % (e.g., no more than 5 wt %, or no more than 2 wt %, or no more than 1 wt %), based on the total weight of the catalyst.
Embodiment 66. A method for making the catalyst of any of embodiments 1-65, the method comprising:
   providing a support that is a cerium oxide support, a titanium oxide support, an aluminum oxide support, a zirconium oxide support, or a mixed oxide support comprising a mixture of two or more of cerium oxide, titanium oxide, aluminum oxide, and zirconium oxide;
   contacting the support with one or more liquids each comprising one or more platinum-, palladium-, or gold-containing compounds and/or one or more manganese-containing compounds dispersed in a solvent(s);
   allowing the solvent(s) to evaporate to provide a catalyst precursor; and calcining the catalyst precursor.
Embodiment 67. The method of embodiment 66, wherein contacting the support with the liquid comprises adding the liquid in an amount equal to the pore volume of the support.
Embodiment 68. The method of embodiment 66, wherein contacting the support with the liquid comprises adding the liquid in an amount greater than the pore volume of the support.
Embodiment 69. The method of any of embodiments 66-68, wherein ratio of the amount liquid to the amount of support on a mass basis is in the range of 1:1 to 5:1 (e.g., in the range of 1:1 to 3:1).
Embodiment 70. The method of any of embodiments 66-69, wherein contacting the support with the liquid provides a slurry.
Embodiment 71. The method of any of embodiments 66-70, wherein allowing the solvent to evaporate is conducted at ambient temperature.
Embodiment 72. The method of embodiments 66-70, wherein allowing the solvent to evaporate is conducted at an elevated temperature (e.g., in the range of 50-150° C.) for a drying time (e.g., 24 hours).
Embodiment 73. The method of embodiments 66-70, wherein allowing the solvent to evaporate is conducted under vacuum and at an elevated temperature (e.g., in the range of 50-150° C.) for a drying time (e.g., 24 hours).
Embodiment 74. The method of any of embodiments 66-70, wherein allowing the solvent to evaporate is conducted in a stirring drybath at an elevated temperature (e.g., in the range of 30-100° C.).
Embodiment 75. The method of any of embodiments 66-74, wherein calcining the catalyst precursor is conducted for a calcining time in the range of 0.5 to 24 hours (e.g., 0.5 to 15 hours, or 0.5 to 10 hours, or 0.5 to 5 hours).
Embodiment 76. The method of any of embodiments 66-75, wherein calcining the catalyst precursor is conducted for a calcining is in the range of 100-600° C. (e.g., in the range of 120-500° C.).
Embodiment 77. The catalyst of any of embodiments 1-65, made by a method according to embodiments 66-76.
Embodiment 78. A method for performing a reverse water-gas shift reaction, the method comprising:
   contacting at a temperature in the range of 200-900° C. a catalyst according to any of embodiments 1-65 and 77 with a feed stream comprising CO₂ and H₂, to provide a product stream comprising CO and H₂, the product stream having a lower concentration of CO₂ and a higher concentration of CO than the feed stream.
Embodiment 79. The method of embodiment 78, wherein the reverse water-gas shift reaction has a CO selectivity of at least 95%, e.g., of at least 96%.
Embodiment 80. The method of embodiment 78, wherein the reverse water-gas shift reaction has a CO selectivity of at least 98%, e.g., of at least 99%.
Embodiment 81. The method of any of embodiments 78-80, wherein the reverse water-gas shift reaction has a methane selectivity of no more than 5%, e.g., no more than 4%.
Embodiment 82. The method of any of embodiments 78-80, wherein the reverse water-gas shift reaction has a methane selectivity of no more than 2%, e.g., no more than 1%.
Embodiment 83. The method of any of embodiments 78-80, wherein the reverse water-gas shift reaction has a methane selectivity of no more than 0.5%, e.g., no more than 0.2%.
Embodiment 84. The method of any of embodiments 78-83, having a CO₂ conversion of at least 5%, e.g., at least 10%, or 20%.
Embodiment 85. The method of any of embodiments 78-83, having a CO₂ conversion of at least 30%, e.g., at least 40%.
Embodiment 86. The method of any of embodiments 78-85, having a CO₂ conversion of no more than 90%, e.g., no more than 80% or no more than 70%.
Embodiment 87. The method of any of embodiments 78-85, having a CO₂ conversion of no more than 65%, e.g., no more than 60%.
Embodiment 88a. The method of any of embodiments 78-87, conducted at a temperature in the range of 200-850° C., e.g., in the range of 200-800° C., or 200-750° C., or 200-700° C., or 200-650° C., or 200-600° C.
Embodiment 88b. The method of any of embodiments 78-87, conducted at a temperature in the range of 250-900° C., e.g., in the range of 250-850° C., or 250-800° C., or 250-700° C., or 250-650° C., or 250-600° C.
Embodiment 89. The method of any of embodiments 78-87, conducted at a temperature in the range of 300-900° C., e.g., in the range of 300-850° C., or 300-800° C., or 300-750° C., or 300-700° C., or 300-650° C., or 300-600° C.
Embodiment 90. The method of any of embodiments 78-87, conducted at a temperature in the range of 350-900° C., e.g., in the range of 350-850° C., or 350-800° C., or 350-750° C., or 350-700° C., or 350-650° C., or 350-600° C.
Embodiment 91. The method of any of embodiments 78-87, conducted at a temperature in the range of 400-900° C., e.g., in the range of 400-850° C., or 400-800° C., or 400-750° C., or 400-700° C., or 400-650° C., or 400-600° C.
Embodiment 92. The method of any of embodiments 78-87, conducted at a temperature in the range of 450-900° C., e.g., in the range of 450-850° C., or 450-800° C., or 450-750° C., or 450-700° C., or 450-650° C., or 450-600° C.
Embodiment 93. The method of any of embodiments 78-87, conducted at a temperature in the range of 500-900° C., e.g., in the range of 500-850° C., or 500-800° C., or 500-750° C., or 500-700° C., or 500-650° C., or 500-600° C.
Embodiment 94. The method of any of embodiments 78-87, conducted at a temperature in the range of 550-900° C., e.g., in the range of 550-850° C., or 550-800° C., or 550-750° C., or 550-700° C., or 550-650° C., or 550-600° C.
Embodiment 95. The method of any of embodiments 78-94, wherein at least part of the H₂ of the feed stream is from a renewable source.
Embodiment 96. The method of any of embodiments 78-95, wherein at least part of the H₂ of the feed stream is green hydrogen.
Embodiment 97. The method of any of embodiments 78-96, wherein at least part of the H₂ of the feed stream is blue hydrogen.
Embodiment 98. The method of any of embodiments 78-97, wherein at least a part of the H₂ of the feed stream is grey hydrogen, black hydrogen, brown hydrogen, pink hydrogen, turquoise hydrogen, yellow hydrogen, and/or white hydrogen.
Embodiment 99. The method of any of embodiments 78-98, wherein at least part of the CO₂ of the feed stream is from a renewable source.
Embodiment 100. The method of any of embodiments 78-99, wherein at least part of the CO₂ of the feed stream is from direct air capture.
Embodiment 101. The method of any of embodiments 78-100, wherein at least part of the CO₂ of the feed stream captured from a manufacturing plant, e.g., a bioethanol plant, a steel plant, or a cement plant.
Embodiment 102. The method of any of embodiments 78-101, wherein the molar ratio of H₂ to CO₂ in the feed stream is at least 0.1:1, e.g., at least 0.5:1.
Embodiment 103. The method of any of embodiments 78-101, wherein the molar ratio of H₂ to CO₂ in the feed stream is at least 0.9:1, e.g., at least 1:1 or at least 1.5:1.
Embodiment 104. The method of any of embodiments 78-101, wherein the molar ratio of H₂ to CO₂ in the feed stream is at least 2:1, e.g., at least 2.5:1.
Embodiment 105. The method of any of embodiments 78-104, wherein the molar ratio of H₂ to CO₂ in the feed stream is no more than 100:1, e.g., no more than 75:1, or 50:1.
Embodiment 106. The method of any of embodiments 78-104, wherein the molar ratio of H₂ to CO₂ in the feed stream is no more than 20:1, e.g., no more than 15:1, or 10:1.
Embodiment 107. The method of any of embodiments 78-104, wherein the molar ratio of H₂ to CO₂ in the feed stream is in the range of 0.5:1 to 10:1.
Embodiment 108. The method of any of embodiments 78-107, conducted at a pressure in the range of 1 to 100 barg (e.g., in the range of 1 to 70 barg, or 1 to 50 barg, or 1 to 40 barg, or 1 to 35 barg, or 5 to 80 barg, or 5 to 50 barg, or 5 to 40 barg, or 5 to 35 barg, or 10 to 70 barg, 10 to 50 barg, or 10 to 40 barg, or 10 to 35 barg, or 20 to 70 barg, 20 to 50 barg, or 20 to 40 barg, or 20 to 35 barg, or 25 to 70 barg, 25 to 50 barg, or 25 to 40 barg, or 25 to 35 barg).
Embodiment 109. The method of any of embodiments 78-108, conducted at a GHSV in the range of 1,000 to 2,000,000 h⁻¹ (e.g., in the range of 1,000 to 1,200,000 h⁻¹, or 1,000 to 500,000 h⁻¹, or 1,000 to 100,000 h⁻¹, or 5,000 to 1,200,000 h⁻¹, or 5,000 to 500,000 h⁻¹, or 5,000 to 100,000 h⁻¹, or 10,000 to 1,200,000 h⁻¹, or 10,000 to 500,000 h⁻¹, or 10,000 to 100,000 h⁻¹).
Embodiment 110. The method of any of embodiments 78-109, wherein the product stream comprises no more than 95 mol % CO₂ (e.g., no more than 90 mol % CO₂).
Embodiment 111. The method of any of embodiments 78-109, wherein the product stream comprises no more than 85 mol % CO₂ (e.g., no more than 80 mol % CO₂).
Embodiment 112. The method of any of embodiments 78-109, wherein the product stream comprises no more than 75 mol % CO₂ (e.g., no more than 70 mol % CO₂).
Embodiment 113. The method of any of embodiments 78-112, wherein the product stream further comprises CO₂, and wherein the method further comprises recycling at least a portion of the CO₂ of the product stream to the feed stream.
Embodiment 114. The method of any of embodiments 78-113, wherein the product stream further comprises hydrogen and wherein the method further comprises recycling at least a portion of the hydrogen of the product stream to the feed stream.
Embodiment 115. The method of any of embodiments 78-114, wherein a ratio of H₂:CO in the product stream is in the range of 0.1:1 to 100:1. (e.g., in the range of 0.1:1 to 50:1, or 0.1:1 to 25:1, or 0.1:1 to 10:1, or 0.1:1 to 5:1, or 1:1 to 100:1, or 1:1 to 50:1, or 1:1 to 25:1, or 1:1 to 10:1, or 1:1 to 5:1).
Embodiment 116. The method of any of embodiments 78-115, wherein the product stream comprises no more than 20 mol % methane, e.g., no more than 15 mol % methane.
Embodiment 117. The method of any of embodiments 78-115, wherein the product stream comprises no more than 10 mol % methane, e.g., no more than 5 mol %, or 1 mol %, or 0.5 mol %, or 0.1 mol % methane.
Embodiment 118. The method of any of embodiments 78-117, wherein the method comprises activating the catalyst prior to contacting the catalyst with the feed stream.
Embodiment 119. The method of embodiment 118, wherein activating the catalyst comprises contacting the catalyst with a reducing stream comprising a reductive gas (e.g., hydrogen).
Embodiment 120. The method of embodiment 118 or embodiment 119, wherein the reducing stream comprises hydrogen in an amount of at least 25 mol % (e.g., at least 50 mol %, or 75 mol %, or 90 mol %).
Embodiment 121. The method of any of embodiments 118-120, wherein activating the catalyst is conducted at a temperature in the range of 200 to 800° C. (e.g., in the range of 250° C. to 800° C., or 300° C. to 800° C., or 200° C. to 700° C., or 250° C. to 800° C., or 300° C. to 700° C.).
Embodiment 122. The method of any of embodiments 118-121, wherein activating the catalyst provides a catalyst that is at least 10% reduced (e.g., at least 25%, or 50%).
Embodiment 123. A process for performing an integrated Fischer-Tropsch process, the process comprising:
   providing a first feed stream comprising H₂ and CO₂;
   contacting at a first temperature in the range of 200-900° C. and at a first pressure a reverse water-gas shift catalyst with the first feed stream to perform a reverse water-gas shift reaction to provide a first product stream comprising CO and H₂, the first product stream having a lower concentration of CO₂ and a higher concentration of CO than the first feed stream;
   contacting at a second temperature and at a second pressure a Fischer-Tropsch catalyst with a second feed stream comprising H₂ and at least a portion of CO of the first product stream to provide a second product stream comprising C₅₊ hydrocarbons,
wherein the reverse water-gas shift catalyst is a supported reverse water-gas shift catalyst comprising:
   a support that is a cerium oxide support, a titanium oxide support, an aluminum oxide support, a zirconium oxide support, or a mixed oxide support comprising a mixture of two or more of cerium oxide, titanium oxide, aluminum oxide, and zirconium oxide;
   at least one of platinum, palladium, and gold present in an amount in the range of 0.05 to 10 wt % of the catalyst, based on the total weight of the catalyst; and
   manganese, present in an amount in the range of 0.5 to 20 wt % of the catalyst, based on the total weight of the catalyst.
Embodiment 124. The process of embodiment 123, wherein the molar ratio of H₂ to CO₂ in the first feed stream is at least 0.1:1, e.g., at least 0.5:1.
Embodiment 125. The process of embodiment 123, wherein the molar ratio of H₂ to CO₂ in the first feed stream is at least 0.9:1, e.g., at least 1:1 or at least 1.5:1.
Embodiment 126. The process of embodiment 123, wherein the molar ratio of H₂ to CO₂ in the first feed stream is at least 2:1, e.g., at least 2.5:1.
Embodiment 127. The process of any of embodiments 1-126, wherein the molar ratio of H₂ to CO₂ in the first feed stream is no more than 100:1, e.g., no more than 75:1, or 50:1.
Embodiment 128. The process of any of embodiments 1-126, wherein the molar ratio of H₂ to CO₂ in the first feed stream is no more than 20:1, e.g., no more than 15:1, or 10:1.
Embodiment 129. The process of any of embodiments 1-128, wherein the molar ratio of H₂ to CO₂ in the first feed stream is in the range of 0.5:1 to 10:1.
Embodiment 130. The process according to any of embodiments 1-129, wherein the first feed stream further comprises CO.
Embodiment 131. The process according to any of embodiments 1-130 wherein the first feed stream further comprises one or more inert gases (e.g., nitrogen and/or methane).
Embodiment 132. The process of any of embodiments 1-131, wherein the reverse water-gas shift reaction has a CO selectivity of at least 70%, e.g., of at least 80%.
Embodiment 133. The process of any of embodiments 1-131, wherein the reverse water-gas shift reaction has a CO selectivity of at least 85%, e.g., or at least 90%.
Embodiment 134. The process of any of embodiments 1-133, wherein the reverse water-gas shift reaction has a CO selectivity of at least 95%, e.g., of at least 96%.
Embodiment 135. The process of any of embodiments 1-133, wherein the reverse water-gas shift reaction has a CO selectivity of at least 98%, e.g., or at least 99%.
Embodiment 136. The process of any of embodiments 1-135, wherein the reverse water-gas shift reaction has a methane selectivity of no more than 5%, e.g., no more than 4%.
Embodiment 137. The process of any of embodiments 1-135, wherein the reverse water-gas shift reaction has a methane selectivity of no more than 2%, e.g., no more than 1%.
Embodiment 138. The process of any of embodiments 1-135, wherein the reverse water-gas shift reaction has a methane selectivity of no more than 0.5%, e.g., no more than 0.2%.
Embodiment 139. The process of any of embodiments 1-138, wherein the reverse water-gas shift reaction has a CO₂ conversion of at least 5%, e.g., at least 10%, or 20%.
Embodiment 140. The process of any of embodiments 1-138, wherein the reverse water-gas shift reaction has a CO₂ conversion of at least 30%, e.g., at least 40%.
Embodiment 141. The process of any of embodiments 1-140, wherein the reverse water-gas shift reaction has a CO₂ conversion of no more than 90%, e.g., no more than 80% or no more than 70%.
Embodiment 142. The process of any of embodiments 1-140, wherein the reverse water-gas shift reaction has a CO₂ conversion of no more than 65%, e.g., no more than 60%.
Embodiment 143. The process of any of embodiments 1-142, wherein the reverse water-gas shift reaction is conducted at a temperature in the range of 200-850° C., e.g., in the range of 200-850° C., or 200-750° C., or 200-700° C., or 200-650° C., or 200-600° C.
Embodiment 144. The process of any of embodiments 1-142, wherein the reverse water-gas shift reaction is conducted at a temperature in the range of 250-900° C., e.g., in the range of 250-850° C., or 250-800° C., or 250-750° C., or 250-700° C., or 250-650° C., or 250-600° C.
Embodiment 145. The process of any of embodiments 1-142, wherein the reverse water-gas shift reaction is conducted at a temperature in the range of 300-900° C., e.g., in the range of 300-850° C., or 300-800° C., or 300-750° C., or 300-700° C., or 300-650° C., or 300-600° C.
Embodiment 146. The process of any of embodiments 1-142, wherein the reverse water-gas shift reaction is conducted at a temperature in the range of 350-900° C., e.g., in the range of 350-850° C., or 350-800° C., or 350-750° C., or 350-700° C., or 350-650° C., or 350-600° C.
Embodiment 147. The process of any of embodiments 1-142, wherein the reverse water-gas shift reaction is conducted at a temperature in the range of 400-900° C., e.g., in the range of 400-850° C., or 400-800° C., or 400-750° C., or 400-700° C., or 400-650° C., or 400-600° C.
Embodiment 148. The process of any of embodiments 1-142, wherein the reverse water-gas shift reaction is conducted at a temperature in the range of 450-900° C., e.g., in the range of 450-850° C., or 450-800° C., or 450-750° C., or 450-700° C., or 450-650° C., or 450-600° C.
Embodiment 149. The process of any of embodiments 1-142, wherein the reverse water-gas shift reaction is conducted at a temperature in the range of 500-900° C., e.g., in the range of 500-850° C., or 500-800° C., or 500-750° C., or 500-700° C., or 500-650° C., or 500-600° C.
Embodiment 150. The process of any of embodiments 1-142, wherein the reverse water-gas shift reaction is conducted at a temperature in the range of 550-900° C., e.g., in the range of 550-850° C., or 550-800° C., or 550-750° C., or 550-700° C., or 550-650° C., or 550-600° C.
Embodiment 151. The process of any of embodiments 1-142, wherein the reverse water-gas shift reaction is conducted at a temperature in the range of 200-500° C., e.g., 200-450° C., or 200-400° C., or 200-350° C., or 250-500° C., e.g., 250-450° C., or 250-400° C., or 250-350° C.
Embodiment 152. The process of any of embodiments 1-151, wherein the reverse water-gas shift reaction is conducted at a pressure in the range of 1 to 100 barg (e.g., in the range of 1 to 70 barg, or 1 to 50 barg, or 1 to 40 barg, or 1 to 35 barg, or 5 to 80 barg, or 5 to 50 barg, or 5 to 40 barg, or 5 to 35 barg, or 10 to 70 barg, 10 to 50 barg, or 10 to 40 barg, or 10 to 35 barg, or 20 to 70 barg, 20 to 50 barg, or 20 to 40 barg, or 20 to 35 barg, or 25 to 70 barg, 25 to 50 barg, or 25 to 40 barg, or 25 to 35 barg).
Embodiment 153. The process of any of embodiments 1-152, wherein the reverse water-gas shift reaction is conducted at a GHSV in the range of 1,000 to 2,000,000 h⁻¹ (e.g., in the range of 1,000 to 1,200,000 h⁻¹, or 1,000 to 500,000 h⁻¹, or 1,000 to 100,000 h⁻¹, or 5,000 to 1,200,000 h⁻¹, or 5,000 to 500,000 h⁻¹, or 5,000 to 100,000 h⁻¹, or 10,000 to 1,200,000 h⁻¹, or 10,000 to 500,000 h⁻¹, or 10,000 to 100,000 h⁻¹).
Embodiment 154. The process of any of embodiments 1-153, wherein the process comprises activating the reverse water-gas shift catalyst, e.g., prior to contacting the reverse water-gas shift catalyst with the first feed stream.
Embodiment 155. The process of embodiment 154, wherein activating the rWGS catalyst comprises contacting the rWGS catalyst with a reducing stream comprising a reductive gas (e.g., hydrogen).
Embodiment 156. The process of embodiment 154 or embodiment 155, wherein the reducing stream comprises hydrogen in an amount of at least 25 mol % (e.g., at least 50 mol %, or 75 mol %, or 90 mol %).
Embodiment 157. The process of any of embodiments 154-156, wherein activating the rWGS catalyst is conducted at a temperature in the range of 200° C. to 800° C. (e.g., in the range of 250° C. to 800° C., or 300° C. to 800° C., or 200° C. to 700° C., or 250° C. to 700° C., or 300° C. to 700° C.).
Embodiment 158. The process of any of embodiments 154-157, wherein activating the rWGS catalyst provides a catalyst that is at least 10% reduced (e.g., at least 25%, or 50%).
Embodiment 159. The process of any of embodiments 1-158, wherein the first product stream comprises no more than 95 mol % CO₂ (e.g., no more than 90 mol % CO₂).
Embodiment 160. The process of any of embodiments 1-158, wherein the first product stream comprises no more than 85 mol % CO₂ (e.g., no more than 80 mol % CO₂).
Embodiment 161. The process of any of embodiments 1-158, wherein the first product stream comprises no more than 75 mol % CO₂ (e.g., no more than 70 mol % CO₂).
Embodiment 162. The process of any of embodiments 1-158, wherein the first product stream comprises in the range of 5-95 mol % CO₂, e.g., 5-90 mol %, or 5-85 mol %, or 5-80 mol %, or 5-75 mol %, or 5-70 mol %, or 10-95 mol %, or 10-90 mol %, or 10-85 mol %, or 10-80 mol %, or 10-75 mol %, or 10-70 mol %, or 20-95 mol %, or 20-90 mol %, or 20-85 mol %, or 20-80 mol %, or 20-75 mol %, or 20-70 mol %, or 30-95 mol %, or 30-90 mol %, or 30-85 mol %, or 30-80 mol %, or 30-75 mol %, or 30-70 mol % CO₂.
Embodiment 163. The process of any of embodiments 1-162, wherein the first product stream comprises no more than 20 mol % methane, e.g., no more than 15 mol % methane.
Embodiment 164. The process of any of embodiments 1-162, wherein the first product stream comprises no more than 10 mol % methane, e.g., no more than 5 mol % or 1 mol %, or 0.5 mol %, or 0.1 mol % methane.
Embodiment 165. The process of any of embodiments 1-164, wherein a ratio of H₂:CO in the first product stream is up to 100:1, e.g., up to 50:1, or up to 25:1, or up to 10:1.
Embodiment 166. The process of any of embodiments 1-164, wherein a ratio of H₂:CO in the first product stream is in the range of 0.1:1 to 100:1 (e.g., in the range of 0.1:1 to 50:1, or 0.1:1 to 25:1, or 0.1:1 to 10:1, or 0.1:1 to 5:1, or 1:1 to 100:1, or 1:1 to 50:1, or 1:1 to 25:1, or 1:1 to 10:1, or 1:1 to 5:1).
Embodiment 167. The process of any of embodiments 1-166, wherein the process further comprises separating the first product stream to recycle at least a portion of one or more components of the first product stream to the first feed stream.
Embodiment 168. The process of any of embodiments 1-167, wherein the process further comprises separating the first product stream to recycle at least a portion (e.g., at least 5 mol %, at least 10 mol %, at least 25 mol %, at least 50 mol %, at least 75 mol %, or at least 90 mol %) of CO₂ of the first product stream to the first feed stream.
Embodiment 169. The process of any of embodiments 1-168, wherein the process further comprises separating the first product stream to recycle at least a portion of H₂ (e.g., at least 5 mol %, at least 10 mol %, at least 25 mol %, at least 50 mol %, at least 75 mol %, or at least 90 mol %) to the first feed stream.
Embodiment 170. The process of any of embodiments 1-169, wherein the process further comprises separating at least a portion of H₂ and/or CO from the first product stream and contacting it with the Fischer-Tropsch catalyst to activate the Fischer-Tropsch catalyst.
Embodiment 171. The process of any of embodiments 1-170, wherein the process further comprises removing at least a portion (e.g., at least 25%, at least 50%, or at least 75%) of water from the first product stream.
Embodiment 172. The process according to any of embodiments 1-171, wherein the first product stream comprises one or more light hydrocarbons (e.g., methane, ethane, propane).
Embodiment 173. The process according to embodiment 172, further comprising separating at least a portion of the one or more light hydrocarbons from the first product stream to provide a light hydrocarbon stream.
Embodiment 174. The process of any of embodiments 1-173, wherein the process further comprises exchanging heat between at least a portion of the first product stream and at least a portion of the first feed stream, thereby cooling at least a portion of the first product stream and heating at least a portion of the first feed stream.
Embodiment 175. The process of any of embodiments 1-174, wherein the process further comprises exchanging heat between at least a portion of the first product stream and a steam generation zone, thereby cooling at least a portion of the first product stream and providing heat to the steam generation zone.
Embodiment 176. The process of embodiment 175, further comprising generating steam from the heat provided to the steam generation zone, and generating electricity from the steam.
Embodiment 177. The process of embodiment 175 or 176, wherein steam is used to heat the first feed stream and/or the second feed stream.
Embodiment 178. The process of any of embodiments 1-177, wherein at least 25% of the CO of the first product stream, e.g., at least 50% of the CO, at least 75% of the CO, or at least 90% of the CO of the first product stream is included in the second feed stream.
Embodiment 179. The process of any of embodiments 1-178, wherein CO is provided to the second feed stream from a CO source other than the first product stream.
Embodiment 180. The process of any of embodiments 1-179, wherein the first product stream includes H₂, and wherein the second feed stream includes at least a portion of the H₂ of the first product stream.
Embodiment 181. The process of any of embodiments 1-180, wherein at least 25% of the H₂ of the first product stream, e.g., at least 50% of the H₂, at least 75% of the H₂, or at least 90% of the H₂ of the first product stream is included in the second feed stream.
Embodiment 182. The process of any of embodiments 1-181, wherein H₂ is provided to the second feed stream from a hydrogen source other than the first product stream.
Embodiment 183. The process of any of embodiments 1-182, wherein the second feed stream includes at least a portion of CO₂ of the first product stream.
Embodiment 184. The process of any of embodiments 1-183, wherein at least 10% of the CO₂ of the first product stream, e.g., at least 25% of the CO₂, at least 50% of the CO₂, at least 75% of the CO₂, or at least 90% of the CO₂ of the first product stream is included in the second feed stream.
Embodiment 185. The process of any of embodiments 1-183, wherein the second feed stream does not include a substantial amount of CO₂ of the first product stream.
Embodiment 186. The process of any of embodiments 1-185, wherein the portion of the first product stream that is included in the second feed stream has a H₂:CO ratio in the range of 0.5:1 to 10:1, e.g., in the range of 1:1 to 3:1.
Embodiment 187. The process of any of embodiments 1-186, wherein the portion of the first product stream that is included in the second feed stream has a water content of no more than 10 mol %, e.g., or no more than 2 mol %, or no more than 0.5 mol %.
Embodiment 188. The process of any of embodiments 1-187, wherein the portion of the first product stream that is included in the second feed stream has a CO₂ content in the range of 10-95 mol % CO₂, e.g., 10-90 mol %, or 10-85 mol %, or 10-80 mol %, or 10-75 mol %, or 10-70 mol %, or 20-95 mol %, or 20-90 mol %, or 20-85 mol %, or 20-80 mol %, or 20-75 mol %, or 20-70 mol %, or 30-95 mol %, or 30-90 mol %, or 30-85 mol %, or 30-80 mol %, or 30-75 mol %, or 30-70 mol % CO₂.
Embodiment 189. The process of any of embodiments 1-188, wherein the second feed stream has a H₂:CO ratio in the range of 0.5:1 to 6:1.
Embodiment 190. The process of any of embodiments 1-188, wherein the second feed stream has a H₂:CO ratio in the range of 1:1 to 3:1, e.g., 1:1 to 2.5:1.
Embodiment 191. The process of any of embodiments 1-188, wherein the second feed stream has a H₂:CO ratio of at least 1.4:1, e.g., in the range of 1.4:1 to 3:1, or 1.4:1 to 2.5:1.
Embodiment 192. The process of any of embodiments 1-191, wherein the second feed stream includes up to 80% of one or more inerts, e.g., up to 70 mol %, up to 60 mol %, or up to 50 mol %, or 15-70 mol %, or 30-70 mol %, or 15-60 mol %, or 30-60 mol %, or 15-50 mol %, or 30-50 mol %.
Embodiment 193. The process of any of embodiments 1-191, wherein the second feed stream includes up to 80% of one or more inerts selected from CO₂, methane and nitrogen, e.g., up to 70 mol %, up to 60 mol %, or up to 50 mol %, or 15-70 mol %, or 30-70 mol %, or 15-60 mol %, or 30-60 mol %, or 15-50 mol %, or 30-50 mol %.
Embodiment 194. The process of any of embodiments 1-193, wherein the second feed stream includes up to 80% of CO₂, e.g., up to 70 mol %, up to 60 mol %, or up to 50 mol %, or 15-70 mol %, or 30-70 mol %, or 15-60 mol %, or 30-60 mol %, or 15-50 mol %, or 30-50 mol %.
Embodiment 195. The process of any of embodiments 1-194, wherein the second feed stream has a water content of no more than 10 mol %, e.g., or no more than 2 mol %, or no more than 0.5 mol %.
Embodiment 196. The process of any of embodiments 1-195, wherein the Fischer-Tropsch catalyst comprises cobalt, iron, rhodium, ruthenium, or a combination thereof.
Embodiment 197. The process of any of embodiments 1-195, wherein the Fischer-Tropsch catalyst comprises cobalt, for example, in an amount in the range of 5-25 wt %, e.g., 7-25 wt %, or 10-25 wt %, or 5-20 wt %, or 7-20 wt %, or 10-20 wt %, calculated as Co(0).
Embodiment 198. The process of any of embodiments 1-195, wherein the Fischer-Tropsch catalyst comprises iron, for example, in an amount in the range of 5-95 wt %, e.g., 10-95 wt %, or 25-95 wt %, or 50-95 wt %, or 5-85 wt %, or 10-85 wt %, or 25-85 wt %, or 50-85 wt %, or 5-75 wt %, or 10-75 wt %, or 25-75 wt %, calculated as Fe(0).
Embodiment 199. The process of any of embodiments 196-198, wherein the Fischer-Tropsch catalyst further comprises manganese.
Embodiment 200. The process of embodiment 199, wherein the manganese is present in an amount up to 15 wt %, e.g., up to 12 wt %, or up to 10 wt %, or up to 7 wt %, or in the range of 0.1-15 wt %, e.g., 0.1-10 wt %, or 0.1-5 wt %, 0.5-15 wt %, or 0.5-10 wt %, or 0.5-5 wt %, calculated as Mn(0).
Embodiment 201. The process of any of embodiments 1-200, wherein the Fischer-Tropsch catalyst is a supported catalyst, wherein the support comprises at least one of titanium oxide, zirconium oxide, cerium oxide, aluminum oxide, silicon oxide and zinc oxide.
Embodiment 202. The process of any of embodiments 1-200, wherein the Fischer-Tropsch catalyst is a supported catalyst, wherein the support comprises at least one of titanium oxide, aluminum oxide, and silicon oxide.
Embodiment 203. The process of any of embodiments 1-200, wherein the Fischer-Tropsch catalyst is a supported catalyst, wherein the support is a titanium dioxide support.
Embodiment 204. The process of any of embodiments 1-203, wherein the Fischer-Tropsch catalyst is activated by contact with a reducing gas, e.g., hydrogen.
Embodiment 205. The process of embodiment 204, wherein the reducing gas comprises at least a portion of hydrogen from the first product stream.
Embodiment 206. The process of any of embodiments 1-203, wherein the Fischer-Tropsch catalyst is activated by contact with H₂ and CO.
Embodiment 207. The process of embodiment 206, wherein the reducing gas comprises at least a portion of H₂ and CO from the first product stream.
Embodiment 208. The process of any of embodiments 204-206, wherein the activation is performed at a temperature in the range of 200-400° C.
Embodiment 209. The process of any of embodiments 1-208, wherein the second temperature is in the range of 150-400° C. (e.g., in the range of 150-350° C., or 150-300° C., or 150-250° C., or 150-200° C., or 200-400° C., or 200-350° C., or 200-300° C., or 200-250° C., or 250-400° C., or 250-350° C., or 250-300° C., or 300-400° C.).
Embodiment 210. The process of any of embodiments 1-209, wherein the second temperature is in the range of 200-350° C.
Embodiment 211. The process of any of embodiments 1-210, wherein the first temperature is within 100° C. of the second temperature, e.g., within 50° C. of the second temperature, or within 25° C. of the second temperature.
Embodiment 212. The process of any of embodiments 1-210, wherein the first temperature is at least 100° C. greater than the second temperature, e.g., at least 150° C. greater than the second temperature, or at least 200° C. greater than the second temperature.
Embodiment 213. The process of any of embodiments 1-212, wherein the second pressure is in the range of 10-50 barg (e.g., 20-50 barg, or 25-50 barg, or 10-40 barg, or 20-40 barg, or 25-40 barg or 10-35 barg, or 20-35 barg, or 25-35 barg).
Embodiment 214. The process of any of embodiments 1-213, wherein the second pressure is in the range of 20-50 barg.
Embodiment 215. The process of any of embodiments 1-214, wherein the Fischer-Tropsch reaction is conducted at a GHSV in the range of 1,000 to 2,000,000 h⁻¹ (e.g., in the range of 1,000 to 1,200,000 h⁻¹, or 1,000 to 500,000 h⁻¹, or 1,000 to 100,000 h⁻¹, or 5,000 to 1,200,000 h⁻¹, or 5,000 to 500,000 h⁻¹, or 5,000 to 100,000 h⁻¹, or 10,000 to 1,200,000 h⁻¹, or 10,000 to 500,000 h⁻¹, or 10,000 to 100,000 h⁻¹).
Embodiment 216. The process of any of embodiments 1-215, wherein the contacting of the Fischer-Tropsch catalyst with the second feed stream to provide the second product stream is performed with a C₅₊ selectivity of at least 30%, e.g., at least 50%, or at least 70%.
Embodiment 217. The process of any of embodiments 1-216, wherein the contacting of the Fischer-Tropsch catalyst with the second feed stream to provide the second product stream is performed with a selectivity for C₅₊ alkanes of at least 30%, e.g., at least 50%, or at least 70%.
Embodiment 218. The process of any of embodiments 1-217, wherein the contacting of the Fischer-Tropsch catalyst with the second feed stream to provide the second product stream is performed with a selectivity for C₅₊ alkanes and C₅₊ alcohols of at least 30%, e.g., at least 50%, or at least 70%.
Embodiment 219. The process of any of embodiments 1-218, further comprising separating at least a portion of water from the second product stream.
Embodiment 220. The process of any of embodiments 1-219, further comprising separating at least a portion of C₁-C₄ hydrocarbons from the second product stream to provide a light hydrocarbon stream.
Embodiment 221. The process of embodiment 220, further comprising including at least a portion of the light hydrocarbon stream in the first feed stream and/or the second feed stream.
Embodiment 222. The process of embodiment 220 or embodiment 221, further comprising oxidizing at least a portion of the light hydrocarbon stream to provide a CO- and/or CO₂-containing pOX stream, and including at least a portion of the pOX stream in the first feed stream and/or the second stream.
Embodiment 223. The process of any of embodiments 220-222, further comprising reforming (e.g., steam reforming and/or autothermal reforming) at least a portion of the light hydrocarbon stream to provide a CO- and/or CO₂-containing reformed stream, and including at least a portion of the reformed stream in the first feed stream and/or the second feed stream.
Embodiment 224. The process of embodiment 222 or embodiment 223, wherein the oxidation or the reforming provides energy, heat energy or electrical energy.
Embodiment 225. The process of any of embodiments 220-224, further comprising burning at least a portion of the light hydrocarbon stream to provide energy, e.g., heat energy or electrical energy.
Embodiment 226. The process of embodiment 225, wherein heat energy is provided, and the heat energy is used to heat the first feed stream.
Embodiment 227. The process of any of embodiments 1-226, wherein the process further comprises exchanging heat between at least a portion of the second product stream and a steam generation zone, thereby cooling at least a portion of the first feed stream and providing heat to the steam generation zone.
Embodiment 228. The process of embodiment 227, further comprising generating steam from the heat provided to the steam generation zone, and generating electricity from the steam.
Embodiment 229. The process of embodiment 227 or 228, wherein steam is used to heat the first feed stream and/or the second feed stream.
Embodiment 230. The process of any of embodiments 1-229, wherein the process further comprises exchanging heat between at least a portion of the second product stream and at least a portion of the second feed stream, thereby cooling at least a portion of the second product stream and heating at least a portion of the second feed stream.
Embodiment 231. The process of any of embodiments 1-230, further comprising recycling at least a portion of H₂ of the second product stream to the second feed stream.
Embodiment 232. The process of any of embodiments 1-231, further comprising recycling at least a portion of H₂ of the second product stream to the first feed stream.
Embodiment 233. The process of embodiment 232, further comprising providing H₂ to the second feed stream from a H₂ source other than the first product stream.
Embodiment 234. The process of embodiment 233, when H₂ from the second product stream makes up most of the H₂ of the first feed stream, e.g., at least 90%, at least 95%, or at least 98% of the H₂ of the first feed stream.
Embodiment 235. The process of any of embodiments 1-234, further comprising recycling at least a portion of CO of the second product stream to the second feed stream.
Embodiment 236. The process of any of embodiments 1-235, further comprising recycling at least a portion of CO of the second product stream to the first feed stream.
Embodiment 237. The process of any of embodiments 1-236, further comprising recycling at least a portion of inerts of the second product stream to the second feed stream.
Embodiment 238. The process of any of embodiments 1-237, further comprising recycling at least a portion of inerts of the second product stream to the first feed stream.
Embodiment 239. The process of any of embodiments 1-238, further comprising recycling at least a portion of CO₂ of the second product stream to the first feed stream.
Embodiment 240. The process of embodiment 239, further comprising providing CO₂ to the second feed stream from a CO₂ source other than the first product stream.
Embodiment 241. The process of embodiment 240, when CO₂ from the second product stream makes up most of the CO₂ of the first feed stream, e.g., at least 90%, at least 95%, or at least 98% of the CO₂ of the first product stream.
Embodiment 242. The process of any of embodiments 1-241, wherein one or more products are provided from at least a portion of C₅₊ hydrocarbons of the second product stream.
Embodiment 243. The process of embodiment 242, wherein the one or more products include fuels (e.g., gasoline, diesel fuel, aviation fuel), lubricants and waxes.
Embodiment 244. The process of any of embodiments 1-243, further comprising hydroprocessing at least a portion of C₅₊ hydrocarbons of the second product stream.
Embodiment 245. The process of any of embodiments 1-244, wherein at least part of the CO₂ of the first feed stream and/or the second feed stream is from a renewable source.
Embodiment 246. The process of any of embodiments 1-245, wherein at least part of the CO₂ of the first feed stream and/or the second feed stream is from direct air capture.
Embodiment 247. The process of any of embodiments 1-246, wherein at least part of the CO₂ of the first feed stream and/or the second feed stream is captured from a manufacturing plant, e.g., a bioethanol plant, a steel plant, or a cement plant.
Embodiment 248. The process of any of embodiments 1-247, wherein at least part of the H₂ of the first feed stream or the second feed stream is from a renewable source.
Embodiment 249. The process of any of embodiment 1-248, wherein at least a portion of the hydrogen of the first feed stream or the second feed stream is green hydrogen.
Embodiment 250. The process of any of embodiment 1-249, wherein at least a portion of the hydrogen of the first feed stream or the second feed stream is blue hydrogen.
Embodiment 251. The process of any of embodiment 1-250, wherein at least a portion of the hydrogen of the first feed stream or the second feed stream is grey hydrogen, black hydrogen, brown hydrogen, pink hydrogen, turquoise hydrogen, yellow hydrogen, and/or white hydrogen.
Embodiment 252. The process of any of embodiments 1-251, further comprising providing at least a portion of H₂ to the first feed stream and/or the second feed stream by electrolysis of water.
Embodiment 253. The process of embodiment 252, wherein the electrolysis of water is performed using at least partially electricity from a renewable source.
Embodiment 254. The process of embodiment 252 or embodiment 253, wherein the electrolysis of water is performed using at least partially electricity generated from steam made by heat exchange from the first product stream and/or the second product stream, or by burning a light hydrocarbon stream.
Embodiment 255. The process of any of embodiments 252-254, further comprising providing at least a portion of O₂ generated in the electrolysis to a partial oxidation.
Embodiment 256. The process of any of embodiments 1-255, wherein the process is performed in a reactor system comprising a first reactor in which the reverse water-gas shift catalyst is disposed, and a second reactor in which the Fischer-Tropsch catalyst is disposed.
Embodiment 257. The process of any of embodiments 1-255, wherein the process is performed in a reactor system comprising first catalyst bed in which the reverse water-gas shift catalyst is disposed, and wherein the second reaction zone comprises a second catalyst bed in which the Fischer-Tropsch catalyst is disposed.
Embodiment 258. The process of embodiment 257, wherein the first reactor bed and the second reactor bed are disposed within the same reactor.
Embodiment 259. The process of any of embodiments 1-255, wherein the process is performed in a reactor system comprising one or more first catalyst containers in which the reverse water-gas shift catalyst is disposed, and wherein the second reaction zone comprises one or more second catalyst containers in which the Fischer-Tropsch catalyst is disposed.
Embodiment 260. The process of embodiment 259, wherein the one or more first catalyst containers and the one or more second catalyst containers are disposed within the same reactor.
Embodiment 261. The process of any of embodiments 1-260, wherein the process is performed in a reactor system comprising a reactor in which the reverse water-gas shift catalyst and the Fischer-Tropsch catalyst are disposed, e.g., in admixture.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed processes and devices are described, it is to be understood that the aspects described herein are not limited to specific embodiments, apparatuses, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand process of referring individually to each separate value falling within the range. Unless otherwise indicated herein, each individual value is incorporated into the specification as if it were individually recited herein. It will be further understood that the endpoints of each of the ranges are significant both in relation to the other endpoint, and independently of the other endpoint.

All processes described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1-41. (canceled)

42. A method for performing a reverse water-gas shift reaction, the method comprising:
contacting at a temperature in the range of 200-900° C. a supported reverse water-gas shift catalyst with a feed stream comprising CO₂ and H₂, to provide a product stream comprising CO and H₂, the product stream having a lower concentration of CO₂ and a higher concentration of CO than the feed stream;
wherein the catalyst comprises:
a support that is a cerium oxide support, a titanium oxide support, an aluminum oxide support, a zirconium oxide support, or a mixed oxide support comprising a mixture of two or more of cerium oxide, titanium oxide, aluminum oxide, and zirconium oxide;
at least one of platinum, palladium, and gold, present in an amount in the range of 0.05 to 10 wt % of the catalyst, based on the total weight of the catalyst; and
manganese, present in an amount in the range of 0.5 to 20 wt % of the catalyst, based on the total weight of the catalyst.

43. The method of claim 42, wherein the reverse water-gas shift reaction has a CO selectivity of at least 95%.

44. The method of claim 42, wherein the reverse water-gas shift reaction has a methane selectivity of no more than 5%.

45. The method of claim 42, having a CO₂ conversion of at least 30%.

46. The method of claim 42, conducted at a temperature in the range of 550-800° C.

47. The method of claim 42, wherein the molar ratio of H₂ to CO₂ in the feed stream is in the range of 0.5:1 to 10:1.

48. The method of claim 42, wherein the product stream further comprises CO₂, and wherein the method further comprises recycling at least a portion of the CO₂ of the product stream to the feed stream, and wherein the product stream further comprises hydrogen and wherein the method further comprises recycling at least a portion of the hydrogen of the product stream to the feed stream.

49. The method of claim 42, wherein the product stream comprises no more than 20 mol % methane.

50. The method of claim 42, wherein platinum is present in the catalyst in an amount in the range of 0.05 to 2 wt %, based on the total weight of the catalyst.

51. The method of claim 42, wherein palladium is present in the catalyst in an amount in a range of 0.05 to 2 wt %, based on the total weight of the catalyst.

52. The method of claim 42, wherein gold is present in the catalyst in an amount in a range of 0.05 to 2 wt %, based on the total weight of the catalyst.

53. The method of claim 42, wherein manganese is present in the catalyst in an amount in the range of 2 to 20 wt %, based on the total weight of the catalyst.

54. A process for performing an integrated Fischer-Tropsch process, the process comprising:
providing a first feed stream comprising H₂ and CO₂;
contacting at a first temperature in the range of 200-900° C. and at a first pressure a reverse water-gas shift catalyst with the first feed stream to perform a reverse water-gas shift reaction to provide a first product stream comprising CO and H₂, the first product stream having a lower concentration of CO₂ and a higher concentration of CO than the first feed stream;
contacting at a second temperature and at a second pressure a Fischer-Tropsch catalyst with a second feed stream comprising H₂ and at least a portion of CO of the first product stream to provide a second product stream comprising C₅₊ hydrocarbons,
wherein the reverse water-gas shift catalyst is a supported reverse water-gas shift catalyst comprising:
a support that is a cerium oxide support, a titanium oxide support, an aluminum oxide support, a zirconium oxide support, or a mixed oxide support comprising a mixture of two or more of cerium oxide, titanium oxide, aluminum oxide, and zirconium oxide;
at least one of platinum, palladium, and gold, present in an amount in the range of 0.05 to 10 wt % of the catalyst, based on the total weight of the catalyst; and
manganese, present in an amount in the range of 0.5 to 20 wt % of the catalyst, based on the total weight of the catalyst.

55. The process of claim 54, wherein the reverse water-gas shift reaction has a methane selectivity of no more than 2%.

56. The process of claim 54, wherein the first product stream comprises no more than 5 mol % methane.

57. The process of claim 54, wherein at least 25% of the CO of the first product stream is included in the second feed stream, and wherein the first product stream includes H₂, and wherein at least 25% of the H₂ of the first product stream is included in the second feed stream.

58. The process of claim 54, wherein at least 10% of the CO₂ of the first product stream is included in the second feed stream.

59. The process of claim 54, wherein the first temperature is at least 100° C. greater than the second temperature.

60. The process of claim 54, wherein the contacting of the Fischer-Tropsch catalyst with the second feed stream to provide the second product stream is performed with a selectivity for C₅₊ alkanes of at least 30%.

61. A supported reverse water-gas shift catalyst comprising:
a support that is a cerium oxide support, a titanium oxide support, an aluminum oxide support, a zirconium oxide support, or a mixed oxide support comprising a mixture of two or more of cerium oxide, titanium oxide, aluminum oxide, and zirconium oxide;
at least one of platinum, palladium, and gold, present in an amount in the range of 0.05 to 10 wt % of the catalyst, based on the total weight of the catalyst; and
manganese, present in an amount in the range of 0.5 to 20 wt % of the catalyst, based on the total weight of the catalyst.
